(19) United States
(12) Patent Application Publication
(10) Pub. No.: US 2022/0251104 A1
(43) Pub. Date: Aug. 11, 2022

(54) AROMATIC COMPOUNDS FOR USE IN ACTIVATING HEMATOPOIETIC STEM AND PROGENITOR CELLS

(71) Applicant: Celularity Inc., Florham Park, NJ (US)

(72) Inventors: Robert J. HARIRI, Bernardsville, NJ (US); Xiaokui ZHANG, Martinsville, NJ (US); James EDINGER, Belford, NJ (US)

(73) Assignee: Celularity Inc., Florham Park, NJ (US)

(21) Appl. No.: 17/297,841

(22) PCT Filed: Nov. 29, 2019
(86) PCT No.: PCT/US2019/063872

§ 371 Date: May 27, 2021

Related U.S. Application Data
(60) Provisional application No. 62/774,101, filed on Nov. 30, 2018.

(51) Int. Cl. C07D 495/04; C07D 491/048; C07D 491/04; C07D 487/04; C07D 473/30; C07D 409/04; C07D 401/12; C07D 401/14

(52) U.S. Cl. CPC C07D 495/04; C07D 491/048; C07D 491/04; C07D 401/14; C07D 473/30; C07D 409/04; C07D 401/12; C07D 487/04

(57) ABSTRACT

Disclosed herein are new aromatic compounds, compositions that include one or more aromatic compounds, and methods of synthesizing the same. Also disclosed herein are methods of increasing and/or expanding cells, including stem cells, hematopoietic stem cells, progenitor cells, and placenta or cord blood-derived cells, with one or more compounds or compositions described herein. Also disclosed herein are methods of increasing and/or expanding differentiated hematopoietic cells with one or more compounds or compositions described herein.

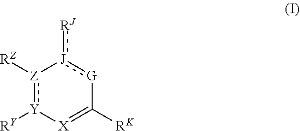

### BACKGROUND

### Field

The present application relates to the fields of chemistry, biochemistry and medicine. More particularly, disclosed herein are new aromatic compounds, compositions that include one or more aromatic compounds, and methods of synthesizing the same. Such compounds can be used to activate biological pathways in cells, particularly hematopoietic stem and progenitor cells to enhance their proliferation and/or expansion in culture.

### Background

Hematopoietic stem and progenitor cells are undifferentiated biological cells that can differentiate into specialized cells and can divide through mitosis to produce more stem and/or progenitor cells. Such cells have the ability to go through numerous cycles of cell division while maintaining an undifferentiated state, and have the capacity to differentiate in specialized cell types. However, there exists an ongoing need to provide expanded populations of hematopoietic stem and progenitor cells in order to make efficient use of the limited number of donor cells. Accordingly, there is a need for compounds and compositions that can increase the expansion and /or proliferation of stem cells and progenitor cells in order to provide the therapeutically effective amounts of both stem and progenitor cells and of differentiated cells derived therefrom necessary for treatment of diseases in humans.

### SUMMARY

Some embodiments disclosed herein relate to a compound of Formula (I), (I-A), (I-B), (I-C), or (I-D), or a pharmaceutically acceptable salt thereof.

Some embodiments disclosed herein relate to a pharmaceutical composition comprising one or more compounds of Formula (I), (I-A), (I-B), (I-C), or (I-D), and one or more pharmaceutically acceptable carriers, diluents, excipients, or combination thereof.

In some embodiments disclosed herein, the stem cells are derived from bone marrow, from placenta or placental perfusate, or from umbilical cord blood. In some embodiments disclosed herein, the stem cells are hematopoietic stem cells.

### BRIEF DESCRIPTION OF THE DRAWINGS

FIG. 1 shows the effects of compounds of Formula (I) on expansion of umbilical cord-derived CD34+ cells.

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, any “R” group(s) such as, without limitation, R^{a}, R^{b}, R^{c}, R^{d}, R^{e}, R^{f}, R^{g}, R^{h}, R^{m}, R^{G}, R^{J}, R^{K}, R^{U}, R^{V}, R^{Y}, and R^{Z} represent substituents that can be attached to the indicated atom. An R group may be substituted or unsubstituted. If two “R” groups are described as being “taken together” the R groups and the atoms they are attached to can form a cycloalkyl, cycloalkenyl, aryl, heteroaryl or heterocycle. For example, without limitation, if R^{a}, and R^{b} of an NR^{a} R^{b} group are indicated to be “taken together,” it means that they are covalently bonded to one another to form a ring:

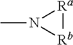

In addition, if two “R” groups are described as being “taken together” with the atom(s) to which they are attached to form a ring as an alternative, the R groups are not limited to the variables or substituents defined previously.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, acylalkyl, hydroxy, alkoxy, alkoxyalkyl, aminoalkyl, amino acid, aryl, heteroaryl, heterocyclyl, aryl(alkyl), heteroaryl(alkyl), heterocyclyl(alkyl), hydroxyalkyl, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, azido, nitro, silyl, sulfenyl, sulfinyl, sulfonyl, haloalkyl, haloalkoxy, trihalomethanesulfonyl, trihalomethanesulfonamido, an amino, a mono-substituted amino group and a di-substituted amino group.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heteroalicyclyl group. That is, the alkyl, alkenyl, alkynyl, ring(s) of the cycloalkyl, ring(s) of the cycloalkenyl, ring(s) of the aryl, ring(s) of the heteroaryl or ring(s) of the heteroalicyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)—and (CH₃)₃C—. If no “a” and “b” are designated with regard to an alkyl, alkenyl, alkynyl, cycloalkyl cycloalkenyl, aryl, heteroaryl or heteroalicyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. Examples of alkenyl groups include allenyl, vinylmethyl and ethenyl. An alkenyl group may be unsubstituted or substituted.

As used herein, “alkynyl” refers to an alkyl group that contains in the straight or branched hydrocarbon chain one or more triple bonds. Examples of alkynyls include ethynyl and propynyl. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s) or 3 to 8 atoms in the ring(s). When composed of two or more rings, the rings may be connected together in a fused fashion. A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic aromatic ring system (a ring system with fully delocalized pi-electron system) that contain(s) one, two, three or more heteroatoms, that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, those described herein and the following: furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur, and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heterocyclyl may be quaternized. Heterocyclyl or heteroalicyclic groups may be unsubstituted or substituted. Examples of such “heterocyclyl” or “heteroalicyclyl” groups include, but are not limited to, those described herein and the following: 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 1,3-thiazinane, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, and 3,4-methylenedioxyphenyl).

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl and naphthylalkyl.

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, imidazolylalkyl and their benzo-fused analogs.

A “heteroalicyclyl(alkyl)” and “heterocyclyl(alkyl)” refer to a heterocyclic or a heteroalicyclylic group connected, as a substituent, via a lower alkylene group. The lower alkylene and heterocyclyl of a heteroalicyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl), and 1,3-thiazinan-4-yl(methyl).

“Lower alkylene groups” are straight-chained —CH₂— tethering groups, forming bonds to connect molecular fragments via their terminal carbon atoms. Examples include but are not limited to methylene (—CH₂—) , ethylene (—CH₂CH₂—) , propylene (—CH₂CH₂CH₂—) , and butylene (—CH₂CH₂CH₂CH₂—) . A lower alkylene group can be substituted by replacing one or more hydrogen of the lower alkylene group with a sub stituent(s) listed under the definition of “substituted.”

As used herein, “alkoxy” refers to the formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) connected, as substituents, via a carbonyl group. Examples include formyl, acetyl, propanoyl, benzoyl and acryl. An acyl may be substituted or unsubstituted.

As used herein, “acylalkyl” refers to an acyl connected, as a substituent, via a lower alkylene group. Examples include aryl-C(═O)—(CH₂)ₙ— and heteroaryl-C(═O)—(CH₂)ₙ—, where n is an integer in the range of 1 to 6.

As used herein, “alkoxyalkyl” refers to an alkoxy group connected, as a substituent, via a lower alkylene group. Examples include C₁₋₄ alkyl-O—(CH₂)ₙ—, wherein n is an integer in the range of 1 to 6.

As used herein, “aminoalkyl” refers to an optionally substituted amino group connected, as a substituent, via a lower alkylene group. Examples include H₂N(CH₂)ₙ—, wherein n is an integer in the range of 1 to 6.

As used herein, “hydroxyalkyl” refers to an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, tri fluoromethyl, chloro-fluoroalkyl, chloro-difluoroalkyl and 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, chloro-fluoroalkyl, chloro-difluoroalkoxy and 2-fluoroisobutoxy. A haloalkoxy may be substituted or unsub stituted.

A “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsub stituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O—” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂—” group wherein each X is a halogen.

A “trihalomethanesulfonamido” group refers to an “X₃CS(O)₂N(R_{A})—” group wherein each X is a halogen, and R_{A} hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl).

The term “amino” as used herein refers to a —H₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “carbonyl” group refers to a C═O group.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

A “urea” group refers to “N(R)—C(═O)—NR_{A}R_{B}” group in which R can be hydrogen or an alkyl, and R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A urea may be substituted or unsubstituted.

The term “halogen atom” or “halogen” as used herein, means any one of the radio-stable atoms of column 7 of the Periodic Table of the Elements, such as, fluorine, chlorine, bromine and iodine.

As used herein, “ [symbol] ” indicates a single or double bond, unless stated otherwise.

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

As used herein, the term “amino acid” refers to any amino acid (both standard and non-standard amino acids), including, but not limited to, α-amino acids, β-amino acids, γ-amino acids and δ-amino acids. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable amino acids include, but are not limited to, ornithine, hypusine, 2-aminoisobutyric acid, dehydroalanine, gamma-aminobutyric acid, citrulline, beta-alanine, alpha-ethyl-glycine, alpha-propyl-glycine and norleucine. As used herein, “amino acid” also includes amino acids wherein the main-chain carboxylic acid group has been converted to an ester group.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

As used herein the terms “stem cells” refers to the cells from which progenitor cells are derived. Stem cells are undifferentiated cells that can differentiate into specialized cells and can divide to produce more stem cells. “Hematopoietic stem cells” refers to cells that can self-renew as well as generate daughter cells of any of the hematopoietic lineages including, but not limited to, T-lymphocytes, B-lymphocytes, natural killer cells, basophil granulocytes, eosinophil granulocytes, neutrophil granulocytes, monocytes, erythrocytes, thrombocytes, and megakaryocytes. Hematopoietic stems cells include cells expressing CD34 (CD34⁺ cells). CD34⁺ cells are normally found in the umbilical cord, placenta, placental perfusate and bone marrow as hematopoietic stem cells.

As used herein, the term “progenitor cells” refers to cells which are precursors of differentiating cells. Most progenitor cells differentiate along a single lineage but they may have extensive proliferative capacity. Progenitor cells appear morphologically as blast cells, and they typically do not have specific features of the hematopoietic lineage to which they are committed.

As used herein, the term “differentiated cells” refers to human hematopoietic cells which have limited or no proliferative capacity. Differentiated cells represent specialized end cells that are found in blood.

As used herein, the term “expansion” refers to an increase in the number of a particular cell type from a starting population of cells, for example, stem cells, hematopoietic stem cells, and progenitor cells.

As used herein, “autologous” refers to cells obtain from the same subject. As used herein, “allogenic” refer to cells of the same species that differ genetically from the cells of the subject.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

Likewise, it is understood that, in any compound described, all tautomeric forms are also intended to be included.

It is to be understood that where compounds disclosed herein have unfilled valencies, then the valencies are to be filled with hydrogens.

Where a range of values is provided, it is understood that the upper and lower limit, and each intervening value between the upper and lower limit of the range is encompassed within the embodiments.

### Compounds

### Formula (I)

Some embodiments disclosed herein relate to a compound of Formula (I), or a pharmaceutically acceptable salt thereof, having the structure:

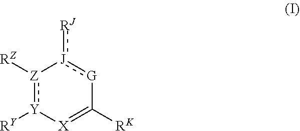

wherein: each [symbol] can independently represent a single bond or a double bond; R^{J} can be selected from the group consisting of —NR^{a}R^{b}, —OR^{b}, and ═O, wherein if R^{J} is ═O, then [symbol] joining G and J represents a single bond and G is N and the N is substituted with R^{G}; otherwise [symbol] joining G and J represents a double bond and G is N; R^{a} can be hydrogen or C₁-C₄ alkyl; R^{b} can be R^{c} or —(C₁-C₄ alkyl)-R^{c}; R^{c} can be selected from the group consisting of: —OH, —O(C₁-C₄ alkyl), —O(C₁-C₄ haloalkyl); —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted can be substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: hydrogen, unsubstituted C₁₋₆ alkyl; substituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted can be substituted with one or more substituents Q, wherein each Q is independently selected from the group consisting of: —OH, C₁₋₄ alkyl, C₁₋₄ haloalkyl, halo, cyano, —O—(C₁₋₄ alkyl), and —O—(C₁₋₄ haloalkyl); R^{G} can be selected from the group consisting of hydrogen, C₁₋₄ alkyl, and —(C₁₋₄ alkyl)-C(═O)NH₂; R^{Y} and R^{Z} can each independently be absent or be selected from the group consisting of: hydrogen, halo, C₁₋₆ alkyl, —OH, —O—(C₁₋₄ alkyl), —NH(C₁₋₄ alkyl), and —N(C₁₋₄ alkyl)₂; or R^{Y} and R^{Z} taken together with the atoms to which they are attached can joined together to form a ring selected from:

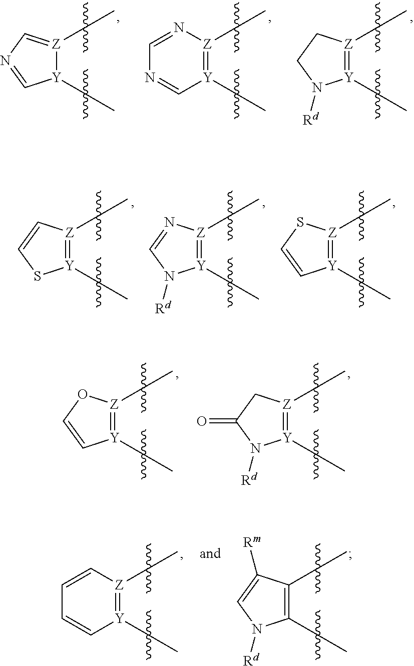

wherein said ring can be optionally substituted with one, two, or three groups independently selected from C₁₋₄ alkyl, C₁₋₄ haloalkyl, halo, cyano, —OH, —O—(C₁₋₄ alkyl), —N(C₁₋₄ alkyl)₂, unsubstituted C₆-C₁₀ aryl, C₆-C₁₀ aryl substituted with 1-5 halo atoms, and —O—(C₁₋₄ haloalkyl); and wherein if R^{Y} and R^{Z} taken together forms

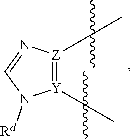

then R^{J} can be —OR^{b} or ═O; R^{d} can be hydrogen or C₁-C₄ alkyl; R^{m} can be selected from the group consisting of C₁₋₄ alkyl, halo, and cyano; J can be C; and X, Y, and Z can each be independently N or C, wherein the valency of any carbon atom is filled as needed with hydrogen atoms.

In some embodiments, [symbol] can represent a single bond. In other embodiments, [symbol] can represent a double bond. In some embodiments, [symbol] joining Y and Z can represent a single bond. In other embodiments, [symbol] joining Y and Z can represent a double bond. In some embodiments, when [symbol] joining G and J representes a single bond, G can be N and the N is substituted with R^{G}. In other embodiments, when [symbol] joining G and J represents a double bond, G can be N. In some embodiments, when [symbol] joining G and J representes a double bond, then [symbol] joining J and R^{J} can be a single bond. In some embodiments, when [symbol] joining G and J representes a double bond, then [symbol] joining J and R^{J} can not be a double bond. In some embodiments, when [symbol] joining J and R^{J} representes a double bond, then [symbol] joining G and J can be a single bond. In some embodiments, when [symbol] joining J and R^{J} representes a double bond, then [symbol] joining G and J can not be a double bond.

In some embodiments, R^{J} can be —NR^{a}R^{b}. In other embodiments, R^{J} can be —OR^{b}. In still other embodiments, R^{J} can be ═O. In some embodiments, when R_{J} is ═O, then [symbol] joining G and J represents a single bond and G is N and the N is substituted with R^{G}. In some embodiments, R^{G} is —CH₂CH₂—C(═O)NH₂.

In some embodiments, R^{a} can be hydrogen. In some embodiments, R^{a} can be C₁-C₄ alkyl. For example, R^{a} can be methyl, ethyl, n-propyl, iso-propyl, n-butyl, iso-butyl or tert-butyl.

In some embodiments, R^{b} can be R^{c}. In some embodiments, R^{b} can be —(C₁-C₄ alkyl)-R^{c}. For example, R^{b} can be —CH₂—R^{c}, —CH₂CH₂—R^{c}, —CH₂CH₂CH₂—R^{c}, or —CH₂CH₂CH₂CH₂—R^{c}. In some embodiments, when R^{b} is —CH₂CH₂—R^{c}, R^{c} can be —O(C₁-C₄ alkyl). In other embodiments, when R^{b} is —CH₂CH₂—R^{c}, R^{c} can be —O(C₁-C₄ haloalkyl). In still other embodiments, when R^{b} is —CH₂CH₂—R^{c}, R^{c} can be —C(═O)NH₂.

In some embodiments, R^{c} can be —OH. In some embodiments, R^{c} can be —O(C₁-C₄ alkyl). In some embodiments, R^{c} can be —O(C₁-C₄ haloalkyl). In some embodiments, R^{c} can be —C(═O)NH₂. In some embodiments, R^{c} can be unsubstituted C₆₋₁₀ aryl. In some embodiments, R^{c} can be substituted C₆₋₁₀ aryl. In some embodiments, R^{c} can be unsubstituted five- to ten-membered heteroaryl having 1-4 atoms selected from the group consisting of O, N, and S. In some embodiments, R^{c} can be substituted five- to ten-membered heteroaryl having 1-4 atoms selected from the group consisting of O, N, and S. In some embodiments, when a R^{c} moiety is indicated as substituted, the moiety can be substituted with one or more, for example, one, two, three, or four substituents E. In some embodiments, E can be —OH. In some embodiments, E can be C₁-C₄ alkyl. In some embodiments, E can be C₁-C₄ haloalkyl. In some embodiments, E can be —O(C₁-C₄ alkyl). In some embodiments, E can be —O(C₁-C₄ haloalkyl).

In some embodiments, when R^{b} is —CH₂CH₂—R^{c}, R^{c} can be unsubstituted C₆₋₁₀ aryl. In other embodiments, when R^{b} is —CH₂CH₂—R^{c}, R^{c} can be substituted C₆₋₁₀ aryl. In still other embodiments, when R^{b} is —CH₂CH₂—R^{c}, R^{c} can be unsubstituted five- to ten-membered heteroaryl having 1-4 atoms selected from the group consisting of O, N, and S. In yet still other embodiments, R^{b} can be —(C₁-C₄ alkyl)-R^{c} and R^{c} can be substituted five- to ten-membered heteroaryl having 1-4 atoms selected from the group consisting of O, N, and S. When a R^{c} moiety is indicated as substituted, the moiety can be substituted with one or more, for example, one, two, three, or four substituents E. In some embodiments, E can be —OH. In other embodiments, E can be C₁-C₄ alkyl. In still other embodiments, E can be C₁-C₄ haloalkyl. In still other embodiments, E can be —O(C₁-C₄ alkyl). In still other embodiments, E can be —O(C₁-C₄ haloalkyl).

In some embodiments, when R^{b} is —CH₂CH₂—R^{c}, R^{c} can be phenyl. In other embodiments, when R^{b} is —CH₂CH₂—R^{c}, R^{c} can be naphthyl. In still other embodiments, when R^{b} is —CH₂CH₂—R^{c}, R^{c} can be hydroxyphenyl. In still other embodiments, when R^{b} is —CH₂CH₂—R^{c}, R^{c} can be indolyl.

In some embodiments, R^{K} can be hydrogen. In other embodiments, R^{K} can be unsubstituted C₁₋₆ alkyl. For example, in some embodiments, R^{K} can be methyl, ethyl, n-propyl, iso-propyl, n-butyl, iso-butyl, tert-butyl, pentyl (branched and straight-chained), or hexyl (branched and straight-chained). In other embodiments, R^{K} can be substituted C₁₋₆ alkyl. In other embodiments, R^{K} can be —NH(C₁₋₄ alkyl). For example, in some embodiments, R^{K} can be —NH(CH₃), —NH(CH₂CH₃), —NH(isopropyl), or —NH(sec-butyl). In other embodiments, R^{K} can be —N(C₁₋₄ alkyl)₂.

In some embodiments, R^{K} can be unsubstituted C₆₋₁₀ aryl. In other embodiments, R^{K} can be substituted C₆₋₁₀ aryl. In other embodiments, R^{K} can be unsubstituted five- to ten-membered heteroaryl having 1-4 atoms selected from the group consisting of O, N, and S. In other embodiments, R^{K} can be substituted five- to ten-membered heteroaryl having 1-4 atoms selected from the group consisting of O, N, and S. When a R^{K} moiety is indicated as substituted, the moiety can be substituted with one or more, for example, one, two, three, or four substituents substituents Q. In some embodiments, Q can be —OH. In other embodiments, Q can be C₁₋₄ alkyl. In still other embodiments, Q can be C₁₋₄ haloalkyl. In still other embodiments, Q can be halo. In still other embodiments, Q can be cyano. In still other embodiments, Q can be —O—(C₁₋₄ alkyl). In still other embodiments, Q can be —O—(C₁₋₄ haloalkyl).

In some embodiments, R^{K} can be phenyl or naphthyl. In other embodiments, R^{K} can be benzothiophenyl. In other embodiments, R^{K} can be benzothiophenyl. In other embodiments, R^{K} can be benzothiophenyl. In still other embodiments, R^{K} can be pyridinyl. In yet still other embodiments, R^{K} can be pyridinyl substituted with one or more substituents Q. For example, R^{K} can be methylpyridinyl, ethylpyridinyl cyanopyridinyl, chloropyridinyl, fluoropyridinyl, or bromopyridinyl.

In some embodiments, R^{G} can be hydrogen. In some embodiments, R^{G} can be C₁₋₄ alkyl. In some embodiments, R^{G} can be —(C₁₋₄ alkyl)-C(═O)NH₂.

In some embodiments, R^{Y} and R^{Z} can independently be absent. In other embodiments, R^{Y} and R^{Z} can independently be hydrogen. In other embodiments, R^{Y} and R^{Z} can independently be halo. In other embodiments, R^{Y} and R^{Z} can independently be C₁₋₆ alkyl. In other embodiments, R^{Y} and R^{Z} can independently be —OH. In still other embodiments, R^{Y} and R^{Z} can independently be —O—(C₁₋₄ alkyl). In other embodiments, R^{Y} and R^{Z} can independently be —NH(C₁₋₄ alkyl). For example, R^{Y} and R^{Z} can independently be —NH(CH₃), —NH(CH₂CH₃), —NH(isopropyl), or —NH(sec-butyl). In other embodiments, R^{Y} and R^{Z} can independently be —N(C₁₋₄ alkyl)₂.

In some embodiments, R^{Y} and R^{Z} taken together with the atoms to which they are attached can be joined together to form a ring. In some embodiments, R^{Y} and R^{Z} taken together with the atoms to which they are attached can be joined together to form

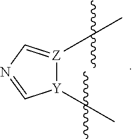

In other embodiments, R^{Y} and R^{Z} taken together with the atoms to which they are attached can be joined together to form

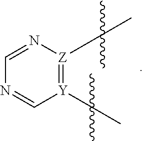

In other embodiments, R^{Y} and R^{Z} taken together with the atoms to which they are attached can be joined together to form

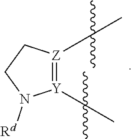

In still other embodiments, R^{Y} and R^{Z} taken together with the atoms to which they are attached can be joined together to form

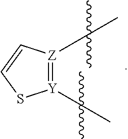

In yet still other embodiments, R^{Y} and R^{Z} taken together with the atoms to which they are attached can be joined together to form

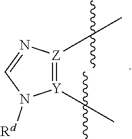

In other embodiments, R^{Y} and R^{Z} taken together with the atoms to which they are attached can be joined together to form

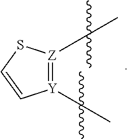

In yet other embodiments, R^{Y} and R^{Z} taken together with the atoms to which they are attached can be joined together to form

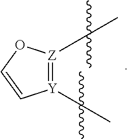

In yet still other embodiments, R^{Y} and R^{Z} taken together with the atoms to which they are attached can be joined together to form

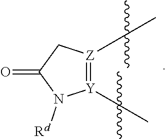

In other embodiments, R^{Y} and R^{Z} taken together with the atoms to which they are attached can be joined together to form

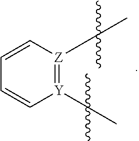

In still other embodiments, R^{Y} and R^{Z} taken together with the atoms to which they are attached can be joined together to form and

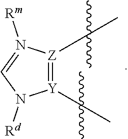

In some embodiments, when R^{Y} and R^{Z} taken together with the atoms to which they are attached can be joined together to form a ring, the ring can be substituted with one, two, or three groups independently selected from C₁-C₄ alkyl, —N(C₁-C₄ alkyl)₂, cyano, unsubstituted phenyl, and phenyl substituted with 1-5 halo atoms.

In some embodiments, when R^{Y} and R^{Z} taken together forms

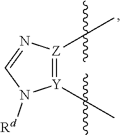

then R^{J} can be —OR^{b} or ═O.

In some embodiments, R^{Y} and R^{Z} taken together with the atoms to which they are attached can be joined together to form

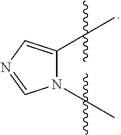 [SMILES: *c1cncn1*]

In other embodiments, R^{Y} and R^{Z} taken together with the atoms to which they are attached can be joined together to form

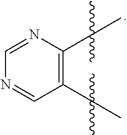 [SMILES: *c1cncnc1*]

In other embodiments, R^{Y} and R^{Z} taken together with the atoms to which they are attached can be joined together to form

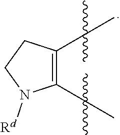

In other embodiments, R^{Y} and R^{Z} taken together with the atoms to which they are attached can be joined together to form

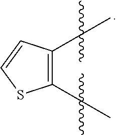 [SMILES: *c1ccsc1*]

In other embodiments, R^{Y} and R^{Z} taken together with the atoms to which they are attached can be joined together to form

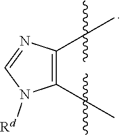

In other embodiments, R^{Y} and R^{Z} taken together with the atoms to which they are attached can be joined together to form

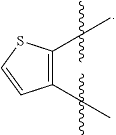 [SMILES: *c1ccsc1*]

In other embodiments, R^{Y} and R^{Z} taken together with the atoms to which they are attached can be joined together to form

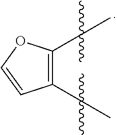 [SMILES: *c1ccoc1*]

In other embodiments, R^{Y} and R^{Z} taken together with the atoms to which they are attached can be joined together to form

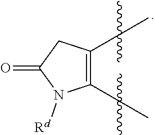

In other embodiments, R^{Y} and R^{Z} taken together with the atoms to which they are attached can be joined together to form

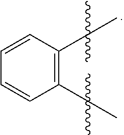 [SMILES: *c1ccccc1*]

In other embodiments, R^{Y} and R^{Z} taken together with the atoms to which they are attached can be joined together to form

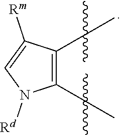

In some embodiments, when R^{Y} and R^{Z} taken together with the atoms to which they are attached can be joined together to form a ring, the ring can be substituted with one, two, or three groups independently selected from C₁-C₄ alkyl, —N(C₁-C₄ alkyl)₂, cyano, unsubstituted phenyl, and phenyl substituted with 1-5 halo atoms. In some embodiments, R^{Y} and R^{Z} taken together with the atoms to which they are attached can be

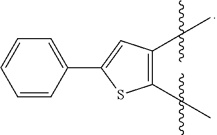 [SMILES: *c1cc(-c2ccccc2)sc1*]

In other embodiments, R^{Y} and R^{Z} taken together with the atoms to which they are attached can be

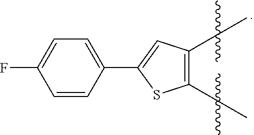 [SMILES: *c1cc(-c2ccc(F)cc2)sc1*]

In still other embodiments, R^{Y} and R^{Z} taken together with the atoms to which they are attached can be

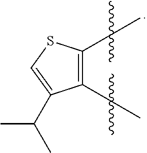 [SMILES: *c1scc(C(C)C)c1*]

In yet still other embodiments, R^{Y} and R^{Z} taken together with the atoms to which they are attached can be

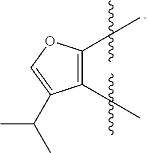 [SMILES: *c1occ(C(C)C)c1*]

In other embodiments, R^{Y} and R^{Z} taken together with the atoms to which they are attached can be

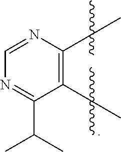 [SMILES: *c1ncnc(C(C)C)c1*]

In some embodiments, R^{d} can be hydrogen. In other embodiments, R^{d} can be C₁-C₄ alkyl. For example R^{d} can be methyl, ethyl, n-propyl, iso-propyl, n-butyl, iso-butyl or tert-butyl. In still other embodiments, R^{d} can be halo. In other embodiments, R^{d} can be cyano.

In some embodiments, R^{m} can be hydrogen. In other embodiments, R^{m} can be C₁-C₄ alkyl. For example R^{m} can be methyl, ethyl, n-propyl, iso-propyl, n-butyl, iso-butyl or tert-butyl. In still other embodiments, R^{m} can be halo. For example, R^{m} can be fluoro, chloro, bromo, or iodo. In other embodiments, R^{m} can be cyano.

In some embodiments, X, Y, and Z can each be independently N or C, wherein the valency of any carbon atom is filled as needed with hydrogen atoms. In some embodiments, X can be N, Y can be N, and Z can be N. In other embodiments, X can be N, Y can be N, and Z can be CH. In some embodiments, X can be N, Y can be CH, and Z can be N. In still other embodiments, X can be CH, Y can be N, and Z can be N. In yet still other embodiments, X can be CH, Y can be CH, and Z can be N. In other embodiments, X can be CH, Y can be N, and Z can be CH. In yet other embodiments, X can be N, Y can be CH, and Z can be CH. In other embodiments, X can be CH, Y can be CH, and Z can be CH.

In some embodiments, R^{a} can be hydrogen; R^{b} can be —(C₁-C₄ alkyl)-R^{c}; R^{c} can be selected from the group consisting of: —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: hydrogen, unsubstituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); R^{G} can be —(C₁₋₄ alkyl)-C(═O)NH₂; R^{Y} and R^{Z} can each be independently absent or be selected from the group consisting of: hydrogen, C₁₋₆ alkyl, and —NH(C₁₋₄ alkyl); or R^{Y} and R^{Z} taken together with the atoms to which they are attached can be joined together to form a ring selected from:

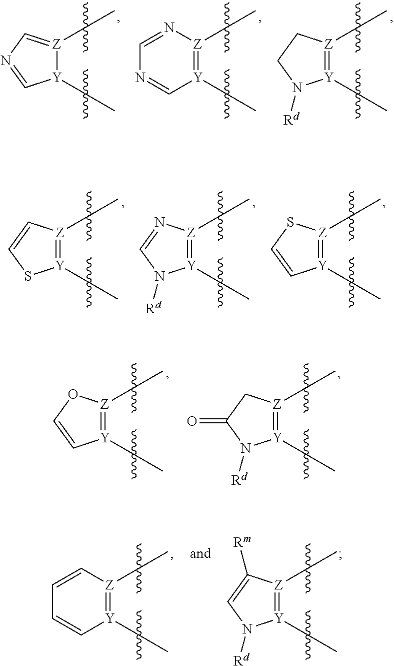

wherein said ring can be optionally substituted with one, two, or three groups independently selected from C₁₋₄ alkyl, C₁₋₄ haloalkyl, halo, cyano, —OH, —O—(C₁₋₄ alkyl), —N(C₁₋₄ alkyl)₂, unsubstituted C₆-C₁₀ aryl, C₆-C₁₀ aryl substituted with 1-5 halo atoms, and —O—(C₁₋₄ haloalkyl); R^{d} can be C₁-C₄ alkyl; R^{m} can be cyano; and X, Y, and Z can each be independently N or C, wherein the valency of any carbon atom is filled as needed with hydrogen atoms.

In some embodiments, R^{a} can be hydrogen; R^{b} can be —CH₂CH₂—R^{c}; R^{c} can be selected from the group consisting of: unsubstituted phenyl, substituted phenyl, indolyl, and —C(═O)NH₂; R^{K} can be selected from the group consisting of: hydrogen, methyl, substituted pyridinyl, unsubstituted benzothiophenyl, and —NH(C₁-C₄ alkyl); R^{G} can be —CH₂CH₂—C(═O)NH₂; R^{Y} can be —NH(C₁-C₄ alkyl); R^{Z} can be absent or hydrogen; or R^{Y} and R^{Z} taken together with the atoms to which they are attached can be joined together to form a ring selected from:

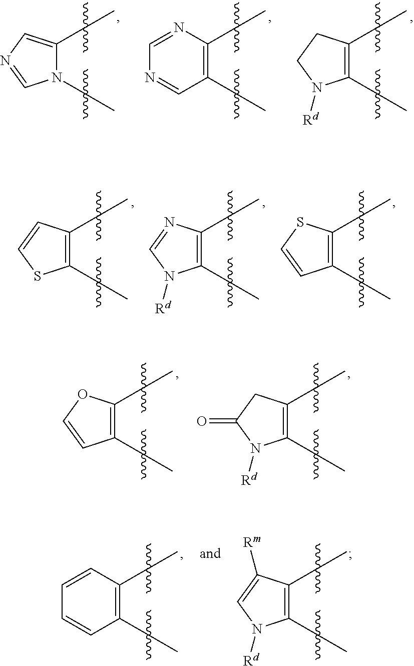

wherein said ring can be optionally substituted with one, two, or three groups independently selected from C₁-C₄ alkyl, —N(C₁-C₄ alkyl)₂, cyano, unsubstituted phenyl, and phenyl substituted with 1-5 halo atoms; R^{d} can be C₁-C₄ alkyl; R^{m} can be cyano; and X can be N or CH.

In some embodiments, when R^{J} is —NR^{a}R^{b}; G can be N; [symbol] joining G and J can be a double bond; R^{a} can hydrogen; R^{b} can be —CH₂CH₂—R^{c}; R^{c} can be unsubstituted five- to ten-membered heteroaryl having 1-4 atoms selected from the group consisting of O, N, and S; or R^{c} can be substituted C₆₋₁₀ aryl, substituted with one or more E, wherein E is —OH; R^{K} can be unsubstituted five- to ten-membered heteroaryl having 1-4 atoms selected from the group consisting of O, N, and S; or R^{K} can be substituted five- to ten-membered heteroaryl having 1-4 atoms selected from the group consisting of O, N, and S; substituted with one or more Q, wherein Q can be selected from cyano, halo, or C₁-C₄ alkyl; R^{Y} and R^{Z} taken together can be

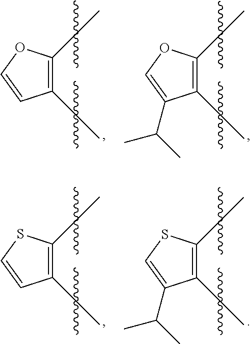

In some embodiments, when R^{J} is —NR^{a}R^{b}; G can be N; [symbol] joining G and J can be a double bond; R^{a} can hydrogen; R^{b} can be —CH₂CH₂—R^{c}; R^{c} can be unsubstituted five- to ten-membered heteroaryl having 1-4 atoms selected from the group consisting of O, N, and S; or R^{c} can be substituted C₆₋₁₀ aryl, substituted with one or more E, wherein E is —OH; R^{K} can be hydrogen, C₁₋₄ alkyl, or unsubstituted five- to ten-membered heteroaryl having 1-4 atoms selected from the group consisting of O, N, and S; and R^{Y} and R^{Z} taken together can be

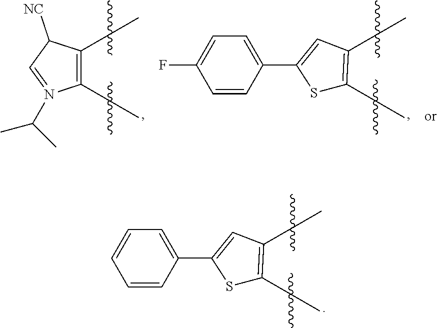

In some embodiments, when R^{J} is —NR^{a}R^{b}; G can be N; [symbol] joining G and J can be a double bond; R^{a} can hydrogen; R^{b} can be —CH₂CH₂—R^{c}; R^{c} can be unsubstituted five- to ten-membered heteroaryl having 1-4 atoms selected from the group consisting of O, N, and S; or R^{c} can be substituted C₆₋₁₀ aryl, substituted with one or more E, wherein E is —OH; R^{K} can be hydrogen, C₁₋₄ alkyl, or unsubstituted five- to ten-membered heteroaryl having 1-4 atoms selected from the group consisting of O, N, and S; and R^{Y} and R^{Z} taken together can be

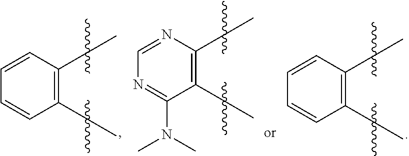 [SMILES: *c1ccccc1* ; *c1ncnc(N(C)C)c1* ; *c1ccccc1*]

In some embodiments, when R^{J} is —NR^{a}R^{b}; G can be N; [symbol] joining G and J can be a double bond, R^{a} can be hydrogen; R^{b} can be —CH₂CH₂—R^{c}; R^{c} can be substituted C₆₋₁₀ aryl; substituted with one or more E, wherein E can be —OH; R^{K} can be unsubstituted five- to ten-membered heteroaryl having 1-4 atoms selected from the group consisting of O, N, and S; R^{Y} can be —NH(C₁₋₄ alkyl); R^{Z} can be hydrogen; J can be C; X can be N; Y can be C; Z can be C; and [symbol] joining Y and Z can be a double bond. In some embodiments, the compound of Formula (I) can be 4-(2-((2-(benzo[b]thiophen-3-yl)-6-(isopropylamino)pyrimidin-4-yl)amino)ethyl)phenol.

In some embodiments, when R^{J} is —NR^{a}R^{b}; G can be N; [symbol] joining G and J can be a double bond; R^{a} can be hydrogen; R^{b} can be —CH₂CH₂—R^{c}, R^{c} can be substituted C₆₋₁₀ aryl, substituted with one or more E, wherein E can be —OH; R^{K} can be unsubstituted five- to ten-membered heteroaryl having 1-4 atoms selected from the group consisting of O, N, and S; R^{Y} and R^{Z} taken together is

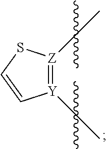

wherein the ring is substituted with C₁-C₄ alkyl; J can be C; X can be N; Y can be C; and Z can be C. In some embodiments, the compound of Formula (I) can be 4-(2-((2-(benzo[b]thiophen-3-yl)-7-isopropylthieno[3,2-d]pyrimidin-4-l)amino)ethyl)phenol.

In some embodiments, when R^{J} is —NR^{a}R^{b}; G can be N; [symbol] joining G and J can be a double bond; R^{a} can be hydrogen; R^{b} can be —CH₂CH₂—R^{c}, R^{c} can be substituted C₆₋₁₀ aryl, substituted with one or more E, wherein E can be —OH; R^{K} can be unsubstituted five- to ten-membered heteroaryl having 1-4 atoms selected from the group consisting of O, N, and S; R^{Y} and R^{Z} taken together is

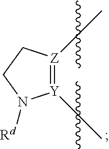

R^{d} can be C₁-C₄ alkyl; J can be C; X can be N; Y can be C; and Z can be C. In some embodiments, the compound of Formula (I) can be 4-(2-((2-(benzo[b]thiophen-3-yl)-7-isopropyl-6,7-dihydro-5H-pyrrolo[2,3-d]pyrimidin-4-yl)amino)ethyl)phenol.

In some embodiments, when R^{J} is —NR^{a}R^{b}; G can be N; [symbol] joining G and J can be a double bond; R^{a} can be hydrogen; R^{b} can be —CH₂CH₂—R^{c}, R^{c} can be substituted C₆₋₁₀ aryl, substituted with one or more E, wherein E can be —OH; R^{K} can be unsubstituted five- to ten-membered heteroaryl having 1-4 atoms selected from the group consisting of O, N, and S; R^{Y} and R^{Z} taken together is

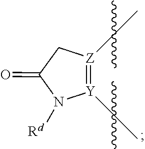

R^{d} can be C₁-C₄ alkyl; J can be C; X can be N; Y can be C; and Z can be C. In some embodiments, the compound of Formula (I) can be 2-(benzo[b]thiophen-3-yl)-4-((4-hydroxyphenethyl)amino)-7-isopropyl-5,7-dihydro-6H-pyrrolo[2,3-d]pyrimidin-6-one.

In some embodiments, when R^{J} is —OR^{b}; G can be N; [symbol] joining G and J can be a double bond; R^{b} can be —CH₂CH₂—R^{c}; R^{c} can be —C(═O)NH₂; R^{K} can unsubstituted five- to ten-membered heteroaryl having 1-4 atoms selected from the group consisting of O, N, and S; R^{Y} and R^{Z} taken together can be

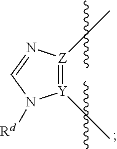

R^{d} can be C₁-C₄ alkyl; J can be C; X can be N; Y can be C; and Z is C. In some embodiments, the compound of Formula (I) can be 3-((2-(benzo[b]thiophen-3-yl)-9-isopropyl-9H-purin-6-yl)oxy)propanamide.

In some embodiments, when R^{J} is is —NR^{a}R^{b}; G can be N; [symbol] joining G and J can be a double bond; R^{b} can be —CH₂CH₂—R^{c}; R^{c} can be substituted C₆₋₁₀ aryl, substituted with one or more E, wherein E is —OH; R^{K} is unsubstituted five- to ten-membered heteroaryl having 1-4 atoms selected from the group consisting of O, N, and S; R^{Y} and R^{Z} taken together can be

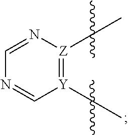

wherein said ring is substituted with —N(C₁₋₄ alkyl)₂; J can be C; X can be N; Y can be C; and Z is C. In some embodiments, the compound of Formula (I) can be 4-(2-((2-(benzo[b]thiophen-3-yl)-8-(dimethylamino)pyrimido[5,4-d]pyrimidin-4-yl)amino)ethyl)phenol.

In some embodiments, when R^{J} is is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is cyano; R^{Y} can be —NH(C₁₋₄ alkyl); R^{Z} can be absent; J can be C; X can be C; Y can be C; Z can be N; and [symbol] joining Y and Z can be a double bond. In some embodiments, the compound of Formula (I) can be 5-(2-((2-(1H-indol-3-yl)ethyl)amino)-6-(sec-butylamino)pyrimidin-4-yl)nicotinonitrile.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be unsubstituted C₁₋₆ alkyl; R^{Y} and R^{Z} taken together can

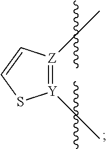

wherein the ring is substituted with unsubstituted C₆-C₁₀ aryl; J can be C; X can be N; Y can be C; Z can be C. In some embodiments, the compound of Formula (I) can be N-(2-(1H-indol-3-yl)ethyl)-2-methyl-6-phenylthieno[2,3-d]pyrimidin-4-amine

In some embodiments, when R^{J} can be —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be hydrogen; R^{Y} and R^{Z} taken together can be

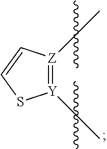

wherein the ring is substituted with substituted C₆-C₁₀ aryl; J can be C; X can be N; Y can be C; and Z can be C. In some embodiments, the compound of Formula (I) can be N-(2-(1H-indol-3-yl)ethyl)-6-(4-fluorophenyl)thieno[2,3-d]pyrimidin-4-amine

In some embodiments, when R^{J} is ═O; G can be N substituted with R^{G}; [symbol] joining G and J can be a single bond; R^{G} can be —(C₁₋₄ alkyl)-C(═O)NH₂; R^{K} can be unsubstituted five- to ten-membered heteroaryl having 1-4 atoms selected from the group consisting of O, N, and S; R^{Y} and R^{Z} taken together can be

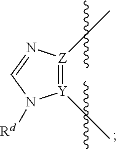

R^{d} can be C₁-C₄ alkyl; J can be C; X can be N; Y can be C; and Z can be C. In some embodiments, the compound of Formula (I) can be 3-(2-(benzo[b]thiophen-3-yl)-9-isopropyl-6-oxo-6,9-dihydro-1H-purin-1-yl)propanamide.

In some embodiments, when R^{J} is —NR^{a}R^{b}; G can be N; [symbol] joining G and J can be a double bond R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q can be halo; R^{Y} and R^{Z} taken together can be

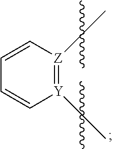

J can be C; X can be N; Y can be C; and Z can be C. In some embodiments, the compound of Formula (I) can be N-(2-(1H-indol-3-yl)ethyl)-2-(5-fluoropyridin-3-yl)quinazolin-4-amine.

In some embodiments, when R^{J} is —NR^{a}R^{b}; G is N; [symbol] joining G and J can be a double bond; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q can be cyano; R^{Y} and R^{Z} taken together is

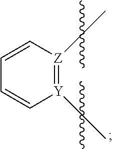

J can be C; X can be N; Y can be C; and Z can be C. In some embodiments, the compound of Formula (I) can be 5-(4-((2-(1H-indol-3-yl)ethyl)amino)quinazolin-2-yl)nicotinonitrile.

In some embodiments, when R^{J} is —NR^{a}R^{b}; G can be N; [symbol] joining G and J can be a double bond; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be —NH(C₁₋₄ alkyl); R^{Y} and R^{Z} taken together can be

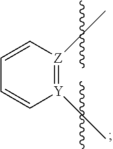

J can be C; X can be N; Y can be C; and Z can be C. In some embodiments, the compound of Formula (I) can be N⁴-(2-(1H-indol-3-yl)ethyl)-N²-(sec-butyl)quinazoline-2,4-diamine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be substituted C₆₋₁₀ aryl, substituted with one or more E, wherein E is —OH; R^{K} can be unsubstituted five- to ten-membered heteroaryl having 1-4 atoms selected from the group consisting of O, N, and S; R^{Y} and R^{Z} taken together can be

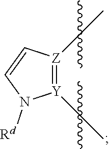

wherein the ring is substituted with cyano; R^{d} can be C₁-C₄ alkyl; J can be C; X can be N; Y can be C; and Z can be C. In some embodiments, the compound of Formula (I) can be 2-(benzo[b]thiophen-3-yl)-4-((4-hydroxyphenethyl)amino)-7-isopropyl-7H-pyrrolo[2,3-d]pyrimidine-5-carbonitrile.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be unsubstituted five- to ten-membered heteroaryl having 1-4 atoms selected from the group consisting of O, N, and S; R^{Y} and R^{Z} taken together can be

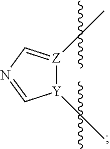

wherein the ring is substituted with C₁₋₄ alkyl; J can be C; X can be C; Y can be N; and Z can be C; wherein the valency of any carbon atom is filled as needed with hydrogen atoms. In some embodiments, the compound of Formula (I) can be N-(2-(1H-indol-3-yl)ethyl)-6-(benzo[b]thiophen-3-yl)-3-isopropylimidazo[1,5-a]pyrazin-8-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be substituted C₆₋₁₀ aryl, substituted with one or more E, wherein E is —OH; R^{K} can be unsubstituted five- to ten-membered heteroaryl having 1-4 atoms selected from the group consisting of O, N, and S; R^{Y} and R^{Z} taken together can be

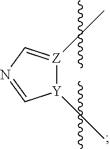

wherein the ring can be substituted with C₁₋₄ alkyl; J can be C; X can be C; Y can be N; and Z can be C; wherein the valency of any carbon atom is filled as needed with hydrogen atoms. In some embodiments, the compound of Formula (I) can be 4-(2-((6-(benzo[b]thiophen-3-yl)-3-isopropylimidazo[1,5-a]pyrazin-8-yl)amino)ethyl)phenol.

In some embodiments, when R^{J} is —NR^{a}R^{b}; G can be N; [symbol] joining G and J represents a double bond; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is cyano; R^{Y} and R^{Z} taken together is

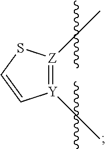

wherein the ring is substituted with C₁-C₄ alkyl; J can be C; X can be N; Y can be C; and Z can be C. In some embodiments, the compound of Formula (I) can be 5-(4-((2-(1H-indol-3-yl)ethyl)amino)-7-isopropylthieno[3,2-d]pyrimidin-2-yl)nicotinonitrile.

In some embodiments, when R^{J} is —NR^{a}R^{b}; G can be N; [symbol] joining G and J represents a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is halo; R^{Y} and R^{Z} taken together can be

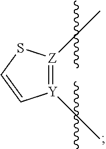

wherein the ring is substituted with C₁-C₄ alkyl; J can be C; X can be N; Y can be C; and Z can be C. In some embodiments, the compound of Formula (I) can be N-(2-(1H-indol-3-yl)ethyl)-2-(5-fluoropyridin-3-yl)-7-isopropylthieno[3,2-d]pyrimidin-4-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is halo; R^{Y} and R^{Z} taken together can be

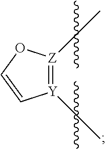

J can be C; X can be N; Y can be C; and Z can be C. In some embodiments, the compound of Formula (I) can be N-(2-(1H-indol-3-yl)ethyl)-2-(5-fluoropyridin-3-yl)furo[3,2-d]pyrimidin-4-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is C₁-C₄ alkyl; R^{Y} and R^{Z} taken together can be

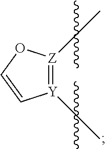

J can be C; X can be N; Y can be C; and Z can be C. In some embodiments, the compound of Formula (I) can be N-(2-(1H-indol-3-yl)ethyl)-2-(5-methylpyridin-3-yl)furo[3,2-d]pyrimidin-4-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is C₁-C₄ alkyl; R^{Y} and R^{Z} taken together can be

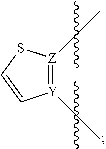

wherein the ring is substituted with C₁-C₄ alkyl J can be C; X can be N; Y can be C; and Z can be C. In some embodiments, the compound of Formula (I) can be N-(2-(1H-indol-3-yl)ethyl)-7-isopropyl-2-(5-methylpyridin-3-yl)thieno[3,2-d]pyrimidin-4-amine.

In some embodiments, when R^{J} is —NR^{a}R^{b}; G is N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is cyano; R^{Y} and R^{Z} taken together can be

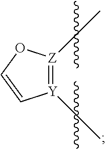

J can be C; X can be N; Y can be C; and Z can be C. In some embodiments, the compound of Formula (I) can be 5-(4-((2-(1H-indol-3-yl)ethyl)amino)furo[3,2-d]pyrimidin-2-yl)nicotinonitrile.

In some emdiments, provided herein is compound of Formula (I), wherein the compound can be selected from:
4-(2-((2-(benzo[b]thiophen-3-yl)-6-(isopropylamino)pyrimidin-4-yl)amino)ethyl)phenol;
4-(2-((2-(benzo[b]thiophen-3-yl)-7-isopropylthieno[3,2-d]pyrimidin-4-yl)amino)ethyl)phenol;
4-(2-((2-(benzo[b]thiophen-3-yl)-7-isopropyl-6,7-dihydro-5H-pyrrolo[2,3-d]pyrimidin-4-yl)amino)ethyl)phenol;
2-(benzo[b]thiophen-3-yl)-4-((4-hydroxyphenethyl)amino)-7-isopropyl-5,7-dihydro-6H-pyrrolo[2,3-d]pyrimidin-6-one;
3-((2-(benzo[b]thiophen-3-yl)-9-isopropyl-9H-purin-6-yl)oxy)propanamide;
4-(2-((2-(benzo[b]thiophen-3-yl)-8-(dimethylamino)pyrimido[5,4-d]pyrimidin-4-yl)amino)ethyl)phenol;
5-(2-((2-(1H-indol-3-yl)ethyl)amino)-6-(sec-butylamino)pyrimidin-4-yl)nicotinonitrile;
N-(2-(1H-indol-3-yl)ethyl)-2-methyl-6-phenylthieno[2,3-d]pyrimidin-4-amine;
N-(2-(1H-indol-3-yl)ethyl)-6-(4-fluorophenyl)thieno[2,3-d]pyrimidin-4-amine;
3-(2-(benzo[b]thiophen-3-yl)-9-isopropyl-6-oxo-6,9-dihydro-1H-purin-1-yl)propanamide;
N-(2-(1H-indol-3-yl)ethyl)-2-(5-fluoropyridin-3-yl)quinazolin-4-amine;
5-(4-((2-(1H-indol-3-yl)ethyl)amino)quinazolin-2-yl)nicotinonitrile;
N⁴-(2-(1H-indol-3-yl)ethyl)-N²-(sec-butyl)quinazoline-2,4-diamine;
2-(benzo[b]thiophen-3-yl)-4-((4-hydroxyphenethyl)amino)-7-isopropyl-7H-pyrrolo[2,3-d]pyrimidine-5-carbonitrile;
N-(2-(1H-indol-3-yl)ethyl)-6-(benzo[b]thiophen-3-yl)-3-isopropylimidazo[1,5-a]pyrazin-8-amine;
4-(2-((6-(benzo[b]thiophen-3-yl)-3-isopropylimidazo[1,5-a]pyrazin-8-yl)amino)ethyl)phenol;
5-(4-((2-(1H-indol-3-yl)ethyl)amino)-7-isopropylthieno[3,2-d]pyrimidin-2-yl)nicotinonitrile;
N-(2-(1H-indol-3-yl)ethyl)-2-(5-fluoropyridin-3-yl)-7-isopropylthieno[3,2-d]pyrimidin-4-amine;
N-(2-(1H-indol-3-yl)ethyl)-2-(5-fluoropyridin-3-yl)furo[3,2-d]pyrimidin-4-amine;
N-(2-(1H-indol-3-yl)ethyl)-2-(5-methylpyridin-3-yl)furo[3,2-d]pyrimidin-4-amine;
N-(2-(1H-indol-3-yl)ethyl)-7-isopropyl-2-(5-methylpyridin-3-yl)thieno[3,2-d]pyrimidin-4-amine;
5-(4-((2-(1H-indol-3-yl)ethyl)amino)furo[3,2-d]pyrimidin-2-yl)nicotinonitrile; and
   pharmaceutically acceptable salts thereof.

### Formula (I-A)

In some embodiments provided herein, the compound of Formula (I) can have the structure of Formula (I-A):

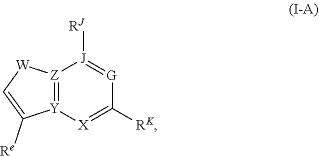

including pharmaceutically acceptable salts thereof, wherein: R^{J} can be NR^{a}R^{b}; R^{a} can be hydrogen or C₁-C₄ alkyl; R^{b} can be R^{c} or —(C₁-C₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: hydrogen, unsubstituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); Y and Z can each be C; X can be N or CH; W can be O or S; and R^{C} can be hydrogen or C₁-C₄ alkyl.

In some embodiments, R^{a} can be hydrogen. In other embodiments, R^{a} can be C₁-C₄ alkyl.

In some embodiments, R^{b} can be —(C₁-C₄ alkyl)-R^{c}. For example, R^{b} can be —CH₂—R^{c}, —CH₂CH₂—R^{c}, —CH₂CH₂CH₂—R^{c}, or —CH₂CH₂CH₂CH₂—R^{c}.

In some embodiments, R^{c} can be —OH. In some embodiments, R^{c} can be —O(C₁-C₄ alkyl). In some embodiments, R^{c} can be —O(C₁-C₄ haloalkyl). In some embodiments, R^{c} can be —C(═O)NH₂. In some embodiments, R^{c} can be unsubstituted C₆₋₁₀ aryl. In some embodiments, R^{c} can be substituted C₆₋₁₀ aryl. In some embodiments, R^{c} can be unsubstituted five- to ten-membered heteroaryl having 1-4 atoms selected from the group consisting of O, N, and S. In some embodiments, R^{c} can be substituted five- to ten-membered heteroaryl having 1-4 atoms selected from the group consisting of O, N, and S. In some embodiments, when a R^{c} moiety is indicated as substituted, the moiety can be substituted with one or more, for example, one, two, three, or four substituents E. In some embodiments, E can be —OH. In some embodiments, E can be C₁-C₄ alkyl. In some embodiments, E can be C₁-C₄ haloalkyl. In some embodiments, E can be —O(C₁-C₄ alkyl). In some embodiments, E can be —O(C₁-C₄ haloalkyl). In some embodiments R^{c} can be phenyl. In other embodiments, R^{c} can be hydroxyphenyl. In still other embodiments, R^{c} can be indolyl.

In some embodiments, R^{K} can be unsubstituted five- to ten-membered heteroaryl having 1-4 atoms selected from the group consisting of O, N, and S. In some embodiments, R^{K} can be substituted five- to ten-membered heteroaryl having 1-4 atoms selected from the group consisting of O, N, and S; wherein the substituted heteroaryl can substituted with one or more substituents Q, wherein each Q can independently selected from the group consisting of: —OH, C₁₋₄ alkyl, C₁₋₄ haloalkyl, halo, cyano, —O—(C₁₋₄ alkyl), and —O—(C₁₋₄ haloalkyl). In some embodiments, R^{K} can be pyridinyl. In other embodiments, R^{K} can be pyridinyl substituted with one or more substituents Q. For example, R^{K} can be methylpyridinyl, ethylpyridinyl cyanopyridinyl, chloropyridinyl, fluoropyridinyl, or bromopyridinyl.

In some embodiments, R^{e} can be hydrogen. In some embodiments, R^{e} can be C₁-C₄ alkyl. For example, R^{e} can be methyl, ethyl, n-propyl, iso-propyl, n-butyl, iso-butyl or tert-butyl.

In some embodiments, R^{a} can be hydrogen; R^{b} can be —(C₁-C₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein the substituted heteroaryl is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); and R^{e} can be C₁-C₄ alkyl.

In some embodiments, R^{a} can be hydrogen; R^{b} can be —(CH₂—CH₂)—R^{c}; R^{c} can be selected from the group consisting of: substituted phenyl and unsubstituted indolyl; wherein the substituted phenyl is substituted with one substituent E, wherein E can be —OH; R^{K} can be selected from the group consisting of: unsubstituted benzothiophenyl and substituted pyridinyl; wherein the substituted pyridinyl is substituted with one substituent Q, wherein Q can be selected from the group consisting of: C₁₋₄ alkyl, halo, and cyano; and R^{e} can be isopropyl.

In some embodiments, when W is O, R^{J} can be —NR^{a}R^{b}; R^{a} can be hydrogen; R^{b} can be —CH₂CH₂—R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, and —O(C₁-C₄ alkyl); R^{K} can be selected from the group consisting of unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —C₁₋₄ alkyl, halo, cyano, and —O—(C₁₋₄ alkyl); Y and Z can each be C; X can be N or CH; and R^{e} can be hydrogen or C₁-C₄ alkyl.

In some embodiments, when W is S, R^{J} can be —NR^{a}R^{b}; R^{a} can be hydrogen; R^{b} can be —CH₂CH₂—R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, and —O(C₁-C₄ alkyl); R^{K} can be selected from the group consisting of unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —C₁₋₄ alkyl, halo, cyano, and —O—(C₁₋₄ alkyl); Y and Z can each be C; X can be N or CH; and R^{e} can be hydrogen or C₁-C₄ alkyl.

In some embodiments, when R^{J} is —NR^{a}R^{b}; G can be N; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is C₁-C₄ alkyl; W can be S; R^{e} can be C₁-C₄ alkyl; J can be C; X can be N; Y can be C; and Z can be C. In some embodiments, the compound of Formula (I-A) can be N-(2-(1H-indol-3-yl)ethyl)-7-isopropyl-2-(5-methylpyridin-3-yl)thieno[3,2-d]pyrimidin-4-amine.

In some embodiments, when R^{J} is —NR^{a}R^{b}; G can be N; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is cyano; W can be S; R^{e} can be C₁-C₄ alkyl; J can be C; X can be N; Y can be C; and Z can be C. In some embodiments, the compound of Formula (I-A) can be 5-(4-((2-(1H-indol-3-yl)ethyl)amino)-7-isopropylthieno[3,2-d]pyrimidin-2-yl)nicotinonitrile.

In some embodiments, when R^{J} is —NR^{a}R^{b}; G can be N; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is halo; W can be S; R^{e} can be C₁-C₄ alkyl; J can be C; X can be N; Y can be C; and Z can be C. In some embodiments, the compound of Formula (I-A) can be N-(2-(1H-indol-3-yl)ethyl)-2-(5-fluoropyridin-3-yl)-7-isopropylthieno[3,2-d]pyrimidin-4-amine.

In some embodiments, when R^{J} is —NR^{a}R^{b}; G can be N; R^{a} can be hydrogen; R^{b} can be —CH₂CH₂—R^{c}, R^{c} can be substituted C₆₋₁₀ aryl, substituted with one or more E, wherein E can be —OH; R^{K} can be unsubstituted five- to ten-membered heteroaryl having 1-4 atoms selected from the group consisting of O, N, and S; W can be S; R^{e} can be C₁-C₄ alkyl; J can be C; X can be N; Y can be C; and Z can be C. In some embodiments, the compound of Formula (I-A) can be 4-(2-((2-(benzo[b]thiophen-3-yl)-7-isopropylthieno[3,2-d]pyrimidin-4-yl)amino)ethyl)phenol.

In some embodiments, when R^{J} is —NR^{a}R^{b}; G can be N; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is halo; W can be O; R^{e} can be hydrogen; J can be C; X can be N; Y can be C; and Z can be C. In some embodiments, the compound of Formula (I-A) can be N-(2-(1H-indol-3-yl)ethyl)-2-(5-fluoropyridin-3-yl)furo[3,2-d]pyrimidin-4-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is C₁-C₄ alkyl; W can be O; R^{e} can be hydrogen; J can be C; X can be N; Y can be C; and Z can be C. In some embodiments, the compound of Formula (I-A) can be N-(2-(1H-indol-3-yl)ethyl)-2-(5-methylpyridin-3-yl)furo[3,2-d]pyrimidin-4-amine.

In some embodiments, when R^{J} is —NR^{a}R^{b}; G is NR^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is cyano; W can be O; R^{c} can be hydrogen; J can be C; X can be N; Y can be C; and Z can be C. In some embodiments, the compound of Formula (I-A) can be 5-(4-((2-(1H-indol-3-yl)ethyl)amino)furo[3,2-d]pyrimidin-2-yl)nicotinonitrile.

In some embodiments, the compound of Formula (I-A), or a pharmaceutically acceptable salt thereof, can selected from the group consisting of:
N-(2-(1H-indol-3-yl)ethyl)-7-isopropyl-2-(5-methylpyridin-3-yl)thieno[3,2-d]pyrimidin-4-amine;
5-(4-((2-(1H-indol-3-yl)ethyl)amino)-7-isopropylthieno[3,2-d]pyrimidin-2-yl)nicotinonitrile;
N-(2-(1H-indol-3-yl)ethyl)-2-(5-fluoropyridin-3-yl)-7-isopropylthieno[3,2-d]pyrimidin-4-amine;
4-(2-((2-(benzo[b]thiophen-3-yl)-7-isopropylthieno[3,2-d]pyrimidin-4-yl)amino)ethyl)phenol;
N-(2-(1H-indol-3-yl)ethyl)-2-(5-fluoropyridin-3-yl)furo[3,2-d]pyrimidin-4-amine;
N-(2-(1H-indol-3-yl)ethyl)-2-(5-methylpyridin-3-yl)furo[3,2-d]pyrimidin-4-amine; and
5-(4-((2-(1H-indol-3-yl)ethyl)amino)furo[3,2-d]pyrimidin-2-yl)nicotinonitrile.

### Formula (I-B)

In other embodiments provided herein, the compound of Formula (I) can have the structure of Formula (I-B):

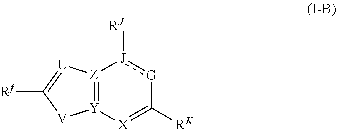

including pharmaceutically acceptable salts thereof, wherein: R^{a} can be hydrogen or C₁-C₄ alkyl; R^{b} can be R^{c} or —(C₁₋₄ alkyl)-R^{c}; R^{c} can be selected from the group consisting of: —OH, —O(C₁-C₄ alkyl), —O(C₁-C₄ haloalkyl); —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: hydrogen, unsubstituted C₁₋₆ alkyl; substituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁-4 alkyl), and —O—(C₁₋₄ haloalkyl); R^{G} can be selected from the group consisting of hydrogen, C₁₋₄ alkyl, and —(C₁₋₄ alkyl)-C(═O)NH₂; R^{f} can be selected from the group consisting of hydrogen, C₁₋₄ alkyl, unsubstituted C₆-C₁₀ aryl, and C₆-C₁₀ aryl substituted with 1-5 halo atoms; U can be N or CR^{U}; V can be S or NR^{V}; R^{U} can be selected from the group consisting of hydrogen, C₁₋₄ alkyl, halo, and cyano; R^{V} can be hydrogen or C₁-C₄ alkyl; wherein when U is CR^{U} and V is NR^{V}, R^{U} is selected from the group consisting of C₁₋₄ alkyl, halo, and cyano; Y and Z can each be C; and X can be N or CH.

In some embodiments, R^{a} can be hydrogen. In other embodiments, R^{a} can be C₁-C₄ alkyl.

In some embodiments, R^{b} can be —(C₁-C₄ alkyl)-R^{c}. For example, R^{b} can be —CH₂—R^{c}, —CH₂CH₂—R^{c}, —CH₂CH₂CH₂—R^{c}, or —CH₂CH₂CH₂CH₂—R^{c}. In certain embodiments, R^{b} can be —(CH₂CH₂)—R^{c}. In certain embodiments, R^{b} can be —(CH₂CH₂)—C(═O)NH₂. In certain embodiments, R^{b} can be —(CH₂CH₂)-(indolyl). In certain embodiments, R^{b} can be —(CH₂CH₂)-(hydroxyphenyl).

In some embodiments, R^{c} can be —OH. In some embodiments, R^{c} can be —O(C₁-C₄ alkyl). In some embodiments, R^{c} can be —O(C₁-C₄ haloalkyl). In some embodiments, R^{c} can be —C(═O)NH₂. In some embodiments, R^{c} can be unsubstituted C₆₋₁₀ aryl. In some embodiments, R^{c} can be substituted C₆₋₁₀ aryl. In some embodiments, R^{c} can be unsubstituted five- to ten-membered heteroaryl having 1-4 atoms selected from the group consisting of O, N, and S. In some embodiments, R^{c} can be substituted five- to ten-membered heteroaryl having 1-4 atoms selected from the group consisting of O, N, and S. In some embodiments, when a R^{c} moiety is indicated as substituted, the moiety can be substituted with one or more, for example, one, two, three, or four substituents E. In some embodiments, E can be —OH. In some embodiments, E can be C₁-C₄ alkyl. In some embodiments, E can be C₁-C₄ haloalkyl. In some embodiments, E can be —O(C₁-C₄ alkyl). In some embodiments, E can be —O(C₁-C₄ haloalkyl).

In some embodiments, R^{K} can be hydrogen. In other embodiments, R^{K} can be C₁-C₄ alkyl. For example, R^{K} can be methyl, ethyl, n-propyl, iso-propyl, n-butyl, iso-butyl or tert-butyl. In some embodiments, R^{K} can be selected from the group consisting of: unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein the substituted heteroaryl can substituted with one or more substituents Q, wherein each Q can independently selected from the group consisting of: —OH, C₁₋₄ alkyl, C₁₋₄ haloalkyl, halo, cyano, —O—(C₁₋₄ alkyl), and —O—(C₁₋₄ haloalkyl). In certain embodiments, R^{K} can be benzothiophenyl. In other embodiments, R^{K} can be pyridinyl substituted with one or more substituents Q. For example, R^{K} can be methylpyridinyl, ethylpyridinyl cyanopyridinyl, chloropyridinyl, fluoropyridinyl, or bromopyridinyl.

In some embodiments, R^{G} can be selected from the group consisting of hydrogen, C₁₋₄ alkyl, and —(C₁₋₄ alkyl)-C(═O)NH₂. In certain embodiments, R^{G} can be —(CH₂CH₂)—C(—O)NH₂.

In some embodiments, R^{f} can be hydrogen. In other embodiments, R^{f} can be C₁₋₄ alkyl. For example, R^{f} can be methyl, ethyl, n-propyl, iso-propyl, n-butyl, iso-butyl or tert-butyl. In some embodiments, R^{f} can be unsubstituted C₆-C₁₀ aryl. In other embodiments, R^{f} can be C₆-C₁₀ aryl substituted with 1-5 halo atoms. In certain embodiments, R^{f} can be phenyl substituted with 1-5 halo atoms. In certain embodiments, R^{f} can be fluorophenyl.

In some embodiments, U can be N. In other embodiments, U can be CR^{U}.

In some embodiments, V can be S. In other embodiments, V can be NR^{V}.

In some embodiments, R^{U} can be hydrogen. In some embodiments, R^{U} can be C₁₋₄ alkyl. In other embodiments R^{U} can be halo. For example, R^{U} can be fluoro, chloro, bromo, or iodo. In still other embodiments, R^{U} can be cyano.

In some embodiments, R^{V} can be hydrogen. In other embodiments, R^{V} can be C₁₋₄ alkyl. For example, R^{V} can be methyl, ethyl, n-propyl, iso-propyl, n-butyl, iso-butyl or tert-butyl. In some embodiments, Y and Z can each be C and X can be N. In other embodiments, Y and Z can each be C and X can be CH.

In some embodiments, R^{a} can be hydrogen; R^{b} can be —(C₁₋₄ alkyl)-R^{c}; R^{c} can be selected from the group consisting of: —C(═O)NH₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted can be substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein the substituted heteroaryl is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); R^{G} is C₁₋₄ alkyl or —(C₁₋₄ alkyl)-C(═O)NH₂; R^{f} can be selected from the group consisting of hydrogen, unsubstituted phenyl, and phenyl substituted with 1-5 halo atoms; Y and Z each can be C; and X can be CH.

In some embodiments, R^{a} can be hydrogen; R^{b} can be —(CH₂—CH₂)—R^{c}; R^{c} can be selected from the group consisting of: —C(═O)NH₂, substituted phenyl and unsubstituted indolyl; wherein the substituted phenyl is substituted with one substituent E, wherein E can be —OH; R^{K} can be selected from the group consisting of: unsubstituted benzothiohenyl and substituted pyridinyl; wherein the substituted pyridinyl is substituted with one substituent Q, wherein Q can be selected from the group consisting of: C₁₋₄ alkyl, halo, and cyano; R^{G} can be —(CH₂CH₂)—C(═O)NH₂; R^{f} can be selected from the group consisting of hydrogen, phenyl, and fluorophenyl; Y and Z each can be C; and X can be CH.

In some embodiments, when V is S, R^{a} can be hydrogen or C₁-C₄ alkyl; R^{b} can be R^{c} or —(CH₂—CH₂)—R^{c}; R^{c} can be selected from the group consisting of: —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, and —O(C₁-C₄ alkyl); R^{K} can be selected from the group consisting of: hydrogen, unsubstituted C₁₋₆ alkyl; substituted C₁₋₆ alkyl; —NH(C₁₋₄ alkyl); and —N(C₁₋₄ alkyl)₂; wherein a R^{K} moiety indicated as substituted is substituted with one or more substituents Q, wherein each Q can be independently selected from the group consisting of: —OH, C₁₋₄ alkyl, halo, cyano, and —O—(C₁₋₄ alkyl; R^{G} can be selected from the group consisting of hydrogen, C₁₋₄ alkyl, and —(C₁₋₄ alkyl)-C(═O)NH₂; R^{f} can be selected from the group consisting of hydrogen, C₁₋₄ alkyl, unsubstituted C₆-C₁₀ aryl, and C₆-C₁₀ aryl substituted with 1-5 halo atoms; U can be CR^{U}; R^{U} can be selected from the group consisting of hydrogen, C₁₋₄ alkyl, halo, and cyano; Y and Z can each be C; and X can be N.

In some embodiments, when V is NR^{V}, R^{a} can be hydrogen or C₁-C₄ alkyl; R^{b} can be R^{c} or —(CH₂—CH₂)—R^{c}; R^{c} can be selected from the group consisting of: —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C_{4,} and —O(C₁-C₄ alkyl); R^{K} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, halo, cyano, and —O—(C₁₋₄ alkyl); R^{G} can be selected from the group consisting of hydrogen, C₁₋₄ alkyl, and —(C₁₋₄ alkyl)-C(═O)NH₂; R^{f} can be hydrogen; U can be N or CR^{U}; R^{U} can be selected from the group consisting of C₁₋₄ alkyl, halo, and cyano; R^{V} can be hydrogen or C₁-C₄ alkyl; Y and Z can each be C; and X can be N or CH.

In some embodiments, when R^{J} is —OR^{b}; G can be N; [symbol] joining G and J can be a double bond; R^{b} can be —CH₂CH₂—R^{c}; R^{c} can be —C(═O)NH₂; R^{K} can unsubstituted five- to ten-membered heteroaryl having 1-4 atoms selected from the group consisting of O, N, and S; U can N; V can be NR^{v}; R^{v} can be C₁-C₄ alkyl; R^{f} can be hydrogen; J can be C; X can be N; Y can be C; and Z can be C. In some embodiments, the compound of Formula (I-B) can be 3-((2-(benzo[b]thiophen-3-yl)-9-isopropyl-9H-purin-6-yl)oxy)propanamide.

In some embodiments, when R^{J} is ═O; G can be N substituted with R^{G}; [symbol] joining G and J can be a single bond; R^{G} can be —(C₁₋₄ alkyl)-C(═O)NH₂; R^{K} can be unsubstituted five- to ten-membered heteroaryl having 1-4 atoms selected from the group consisting of O, N, and S; U can N; V can be NR^{v}; R^{v} can be C₁-C₄ alkyl; R^{f} can be hydrogen; J can be C; X can be N; Y can be C; and Z can be C. In some embodiments, the compound of Formula (I-B) can be 3-(2-(benzo[b]thiophen-3-yl)-9-isopropyl-6-oxo-6,9-dihydro-1H-purin-1-yl)propanamide.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be substituted C₆₋₁₀ aryl, substituted with one or more E, wherein E is —OH; R^{K} can be unsubstituted five- to ten-membered heteroaryl having 1-4 atoms selected from the group consisting of O, N, and S; U can be CR^{u}; R^{u} can be cyano; V can be NR^{v}R^{v} can be C₁-C₄ alkyl; R^{f} can be hydrogen; J can be C; X can be N; Y can be C; and Z can be C. In some embodiments, the compound of Formula (I-B) can be 2-(benzo[b]thiophen-3-yl)-4-((4-hydroxyphenethyl)amino)-7-isopropyl-7H-pyrrolo[2,3-d]pyrimidine-5-carbonitrile.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be unsubstituted C₁₋₆ alkyl; U can be CR^{u}; R^{u} can be hydrogen; V can be S; R^{f}can be phenyl; J can be C; X can be N; Y can be C; Z can be C. In some embodiments, the compound of Formula (I-B) can be N-(2-(1H-indol-3-yl)ethyl)-2-methyl-6-phenylthieno[2,3-d]pyrimidin-4-amine.

In some embodiments, when R^{J} can be —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be hydrogen; U can be CR^{u}; R^{u} can be hydrogen; V can be S; R^{f} can be fluorophenyl; J can be C; X can be N; Y can be C; and Z can be C. In some embodiments, the compound of Formula (I-B) can be N-(2-(1H-indol-3-yl)ethyl)-6-(4-fluorophenyl)thieno[2,3-d]pyrimidin-4-amine.

In some embodiments, the compound of Formula (I-B), or a pharmaceutically acceptable salt thereof, can selected from the group consisting of:
3-((2-(benzo[b]thiophen-3-yl)-9-isopropyl-9H-purin-6-yl)oxy)propanamide;
3-(2-(benzo[b]thiophen-3-yl)-9-isopropyl-6-oxo-6,9-dihydro-1H-purin-1-yl)propanamide;
2-(benzo[b]thiophen-3-yl)-4-((4-hydroxyphenethyl)amino)-7-isopropyl-7H-pyrrolo[2,3-d]pyrimidine-5-carbonitrile;
N-(2-(1H-indol-3-yl)ethyl)-2-methyl-6-phenylthieno[2,3-d]pyrimidin-4-amine; and
N-(2-(1H-indol-3-yl)ethyl)-6-(4-fluorophenyl)thieno[2,3-d]pyrimidin-4-amine.

### Formula (I-C)

In still other embodiments provided herein, the compound of Formula (I) can have the structure of Formula (I-C):

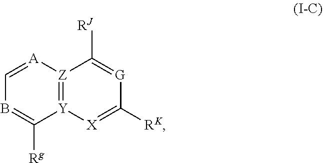

including pharmaceutically acceptable salts thereof, wherein: R^{J} can be —NR^{a}R^{b}; R^{a} can be hydrogen or C₁-C₄ alkyl; R^{b} can be R^{c} or —(C₁-C₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: hydrogen, unsubstituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); A can be N or CH; B can be N or CH; R^{g} can be selected from the group consisting of hydrogen, C₁₋₄ alkyl, and —N(C₁₋₄ alkyl)₂; Y and Z can each be C; and X can be N or CH.

In some embodiments, R^{K} can be —NH(C₁₋₄ alkyl). For example, in some embodiments, R^{K} can be —NH(CH₃), —NH(CH₂CH₃), —NH(isopropyl), or —NH(sec-butyl). In some embodiments, R^{K} can be unsubstituted benzothiophenyl. In other embodiments, R^{K} can be substituted pyridinyl. For example, R^{K} can be methylpyridinyl, ethylpyridinyl, cyanopyridinyl, chloropyridinyl, fluoropyridinyl, or bromopyridinyl.

In some embodiments, A can be N and B can be N. In other embodiments, A can be N and B can be CH. In still other embodiments, A can be CH and B can be N. In yet still other embodiments, A can be CH and B can be CH.

In some embodiments, R^{g} can be hydrogen. In other embodiments, R^{g} can be —N(C₁₋₄ alkyl)₂. In certain embodiments, R^{g} can be —N(CH₃)₂.

In some embodiments, R^{a} can be hydrogen; R^{b} can be —(C₁-C₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: —NH(C₁₋₄ alkyl); unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein the substituted heteroaryl is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); and R^{g} can be hydrogen or —N(C₁₋₄ alkyl)₂.

In some embodiments, R^{a} can be hydrogen; R^{b} can be —(C₁-C₄ alkyl)-R^{c}; R^{c} can be selected from the group consisting of: substituted phenyl and unsubstituted indolyl; wherein the substituted phenyl is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: —NH(C₁₋₄ alkyl); unsubstituted benzothiophenyl; and substituted pyridinyl; wherein the substituted pyridinyl is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); and R^{g} can be hydrogen or —N(C₁₋₄ alkyl)₂.

In some embodiments, R^{a} can be hydrogen; R^{b} can be —(CH₂CH₂)—R^{c}; R^{c} can be selected from the group consisting of: substituted phenyl and unsubstituted indolyl; wherein the substituted phenyl is substituted with one substituent E, wherein E can be —OH; R^{K} can be selected from the group consisting of: —NH(sec-butyl); unsubstituted benzothiohenyl, and substituted pyridinyl; wherein the substituted pyridinyl is substituted with one or more substituents Q, wherein each Q can be independently selected from the group consisting of: C₁₋₄ alkyl, halo, and cyano; and R^{g} can be hydrogen or —N(CH₃)₂.

In some embodiments, when A is C and B is C, R^{J} can be —NR^{a}R^{b}; G can be N; R^{a} can be hydrogen; R^{b} can be —CH₂CH₂—R^{c}; R^{c} can be substituted C₆₋₁₀ aryl, substituted with one or more E, wherein E is —OH; or unsubstituted five- to ten-membered heteroaryl having 1-4 atoms selected from the group consisting of O, N, and S; R^{K} can be unsubstituted five- to ten-membered heteroaryl having 1-4 atoms selected from the group consisting of O, N, and S; R^{g} can be hydrogen; J can be C; X can be N; Y can be C; and Z is C.

In some embodiments, when R^{J} is —NR^{a}R^{b}; G can be N; R^{a} can be hydrogen; R^{b} can be —CH₂CH₂—R^{c}R^{c}; R^{c} can be substituted C₆₋₁₀ aryl, substituted with one or more E, wherein E is —OH; R^{K} is unsubstituted five- to ten-membered heteroaryl having 1-4 atoms selected from the group consisting of O, N, and S; A can be N; B can be N; R^{g} can be —N(C₁₋₄ alkyl)₂; J can be C; X can be N; Y can be C; and Z is C. In some embodiments, the compound of Formula (I-C) can be 4-(2-((2-(benzo[b]thiophen-3-yl)-8-(dimethylamino)pyrimido[5,4-d]pyrimidin-4-yl)amino)ethyl)phenol.

In some embodiments, when R^{J} is —NR^{a}R^{b}; G can be N; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q can be halo; A can be CH; B can be CH; R^{g} can be hydrogen; J can be C; X can be N; Y can be C; and Z can be C. In some embodiments, the compound of Formula (I-C) can be N-(2-(1H-indol-3-yl)ethyl)-2-(5-fluoropyridin-3-yl)quinazolin-4-amine.

In some embodiments, when R^{J} is —NR^{a}R^{b}; G is N; [symbol] joining G and J can be a double bond; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q can be cyano; A can be CH; B can be CH; R^{g} can be hydrogen; J can be C; X can be N; Y can be C; and Z can be C. In some embodiments, the compound of Formula (I-C) can be 5-(4-((2-(1H-indol-3-yl)ethyl)amino)quinazolin-2-yl)nicotinonitrile.

In some embodiments, when R^{J} is —NR^{a}R^{b}; G can be N; [symbol] joining G and J can be a double bond; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be —NH(C₁₋₄ alkyl); A can be CH; B can be CH; R^{g} can be hydrogen; J can be C; X can be N; Y can be C; and Z can be C. In some embodiments, the compound of Formula (I-C) can be N⁴-(2-(1H-indol-3-yl)ethyl)-N²-(sec-butyl)quinazoline-2,4-diamine.

In some embodiments, the compound of Formula (I-C), or a pharmaceutically acceptable salt thereof, can selected from the group consisting of:
4-(2-((2-(benzo[b]thiophen-3-yl)-8-(dimethylamino)pyrimido[5,4-d]pyrimidin-4-yl)amino)ethyl)phenol;
N-(2-(1H-indol-3-yl)ethyl)-2-(5-fluoropyridin-3-yl)quinazolin-4-amine;
5-(4-((2-(1H-indol-3-yl)ethyl)amino)quinazolin-2-yl)nicotinonitrile; and
N⁴-(2-(1H-indol-3-yl)ethyl)-N²-(sec-butyl)quinazoline-2,4-diamine.

### Formula (I-D)

In yet still other embodiments provided herein, the compound of Formula (I) can have the structure of Formula (I-D):

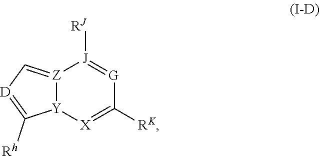

including pharmaceutically acceptable salts thereof, wherein: R^{J} can be —NR^{a}R^{b}; R^{a} can be hydrogen or C₁-C₄ alkyl; R^{b} can be R^{c} or —(C₁₋₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); R^{h} can be hydrogen or C₁₋₄ alkyl; D can be N or CH; Y can be N; Z can be C; and X can be N or CH.

In some embodiments, R^{h} can be hydrogen. In other embodiments, R^{h} can be C₁₋₄ alkyl. For example, R^{h} can be methyl, ethyl, n-propyl, iso-propyl, n-butyl, iso-butyl or tert-butyl.

In some embodiments, D can be N. In other embodiments, D can be CH.

In some embodiments, when D is N, Y can be N, Z can be C, and X can be N. In other embodiments, when D is N, Y can be N, Z can be C, and X can be CH. In some embodiments, when D is CH, Y can be N, Z can be C, and X can be N. In other embodiments, when D is CH, Y can be N, Z can be C, and X can be CH.

In some embodiments, R^{a} can be hydrogen; R^{b} can be —(C₁₋₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁-4 alkyl), and —O—(C₁₋₄ haloalkyl); and R^{h} can be hydrogen or C₁₋₄ alkyl.

In some embodiments, R^{a} can be hydrogen; R^{b} can be —(C₁-C₄ alkyl)-R^{c}; R^{c} can be selected from the group consisting of: substituted phenyl and unsubstituted indolyl; wherein the substituted phenyl is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be unsubstituted benzothiophenyl; and R^{h} can be hydrogen or C₁₋₄ alkyl.

In some embodiments, R^{a} can be hydrogen; R^{b} can be —(CH₂—CH₂)—R^{c}; R^{c} can be selected from the group consisting of: substituted phenyl and unsubstituted indolyl; wherein the substituted phenyl is substituted with one substituent E, wherein E can be —OH; R^{K} can be unsubstituted benzothiophenyl; and ER^{h} can be hydrogen or C₁₋₄ alkyl.

In some embodiments, when D is N; R^{J} is —NR^{a}R^{b}; G can be N; R^{a} can be hydrogen; R^{b} can be —CH₂CH₂—R^{c}; R^{c} can be unsubstituted five- to ten-membered heteroaryl having 1-4 atoms selected from the group consisting of O, N, and S; or substituted C₆₋₁₀ aryl, substituted with one or more E, wherein E is —OH; R^{K} can be unsubstituted five- to ten-membered heteroaryl having 1-4 atoms selected from the group consisting of O, N, and S; R^{h} can be C₁₋₄ alkyl; J can be C; X can be C; Y can be N; and Z can be C; wherein the valency of any carbon atom is filled as needed with hydrogen atoms.

In some embodiments, when R^{J} is —NR^{a}R^{b}; G can be N; R^{a} can be hydrogen; R^{b} can be —CH₂CH₂—R^{c}; R^{c} can be unsubstituted five- to ten-membered heteroaryl having 1-4 atoms selected from the group consisting of O, N, and S or substituted C₆₋₁₀ aryl, substituted with one or more E, wherein E is —OH; R^{K} can be unsubstituted five- to ten-membered heteroaryl having 1-4 atoms selected from the group consisting of O, N, and S; D can be N; R^{h} can be C₁₋₄ alkyl; J can be C; X can be C; Y can be N; and Z can be C; wherein the valency of any carbon atom is filled as needed with hydrogen atoms. In some embodiments, the compound of Formula (I-D) can be N-(2-(1H-indol-3-yl)ethyl)-6-(benzo[b]thiophen-3-yl)-3-sopropylimidazo[1,5-a]pyrazin-8-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be substituted C₆₋₁₀ aryl, substituted with one or more E, wherein E is —OH; R^{K} can be unsubstituted five- to ten-membered heteroaryl having 1-4 atoms selected from the group consisting of O, N, and S; D can be N; R^{h} can be C₁₋₄ alkyl; J can be C; X can be C; Y can be N; and Z can be C; wherein the valency of any carbon atom is filled as needed with hydrogen atoms. In some embodiments, the compound of Formula (I-D) can be 4-(2-((6-(benzo[b]thiophen-3-yl)-3-isopropylimidazo[1,5-a]pyrazin-8-yl)amino)ethyl)phenol.

In some embodiments, the compound of Formula (I-D), or a pharmaceutically acceptable salt thereof, can selected from the group consisting of: N-(2-(1H-indol-3-yl)ethyl)-6-(benzo[b]thiophen-3-yl)-3-sopropylimidazo[1,5-a]pyrazin-8-amine; and 4-(2((6-(benzo[b]thiophen-3-yl)-3-isopropylimidazo[1,5-a]pyrazin-8-yl)amino)ethyl)phenol.

### Synthesis

Compounds of Formula (I), (I-A), (I-B), (I-C), or (I-D) and those described herein may be prepared in various ways. General synthetic routes to the compounds of Formula (I), (I-A), (I-B), (I-C), or (I-D), and some examples of starting materials used to synthesize the compounds of Formula (I), (I-A), (I-B), (I-C), or (I-D) are shown and described herein. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

### Pharmaceutical Compositions

Some embodiments described herein relate to a pharmaceutical composition, that can include an effective amount of one or more compounds described herein (e.g., a compound of Formula (I), (I-A), (I-B), (I-C), or (I-D), or a pharmaceutically acceptable salt thereof) and a pharmaceutically acceptable carrier, diluent, excipient or combination thereof.

The term “pharmaceutical composition” refers to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

The term “physiologically acceptable” defines a carrier, diluent or excipient that does not abrogate the biological activity and properties of the compound nor cause appreciable damage or injury to an animal to which delivery of the composition is intended.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound exist in the art including, but not limited to, oral, rectal, pulmonary, topical, aerosol, injection and parenteral delivery, including intramuscular, subcutaneous, intravenous, intramedullary injections, intrathecal, direct intraventricular, intraperitoneal, intranasal and intraocular injections.

One may also administer the compound in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory infection may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound described herein formulated in a compatible pharmaceutical carrier may also be prepared, placed in an appropriate container, and labeled for treatment of an indicated condition.

Methods of Use

Some embodiments described herein relate to a method of using a compound of Formula (I), (I-A), (I-B), (I-C), or (I-D), or pharmaceutically acceptable salts thereof to stimulate the expansion of cells. In some embodiments, the method comprises contacting cells with a compound of Formula (I), (I-A), (I-B), (I-C), or (I-D). In some embodiments, the expansion of cells using a compound of Formula (I), (I-A), (I-B), (I-C), or (I-D), or pharmaceutically acceptable salts, results in an increase in the number of cells. In some embodiments, the number of cells may be increased by increasing the number of cell divisions.

In some embodiments, the cells may be stem cells, for example hematopoietic stem cells. In other embodiments, the cells may be progenitor cells. In some embodiments, the method of cell expansion may be an in vivo method. In some embodiments, the method of cell expansion may be an in vitro method. In some embodiments, the method of cell expansion may be an ex vivo method.

Expansion of cell populations can provide cells for treatment of a variety of diseases of disorders. For many of these treatments, a relatively large number of cells should be isolated. The number of available cells is often a clinical limitation for procedures such as cell transplantation. For example, it is estimated that about a quarter of autologous donor stem cell transplants cannot be performed due to insufficient availability of cells. Moreover, fewer than 25% of patients that need allopathic stem cell transplants can find a suitable donor. Compounds of Formula (I), (I-A), (I-B), (I-C), or (I-D) can be used to expand the number of stem cells. Additionally, the compounds of Formula (I), (I-A), (I-B), (I-C), or (I-D) can be used to increase the number of other clinically useful cells including but not limited to progenitor cells and differentiated cells, such as differentiated hematopoietic cells.

In some embodiments, compounds of Formula (I), (I-A), (I-B), (I-C), or (I-D), or a pharmaceutically acceptable salt thereof, can be used to expand cell populations for transplantation to a subject. In some embodiments, the transplantation may be a autologous transplantation. In other embodiments, the transplantation may be an allogenic transplantation. In some embodiments, the diseases or disorders that may be treated by cell transplantation include, but are not limited to, acute lymphoblastic leukemia, acute myelofibrosis, acute myeloid leukemia, acute undifferentiated leukemia, adrenoleukodystrophies, amyloidosis, amyotrophic lateral sclerosis, aplastic anemia, Alzheimer's disease, ataxia, cerebral palsy, chronic lymphocytic leukemia, chronic myeloid leukemia, chronic obstructive pulmonary disease, coronary artery disease, diabetes-type 1, Crohn's disease, Fanconi anemia, fibromyalgia, Gaucher disease germ cell tumors, Graft-versus-Host disease, hemophagocytic lymphohistiocytosis, Hodgkin disease, Hurler's Syndrome, kidney disease, Krabbe's disease, liver disease, metachromatic leukodystrophiesm, mucopolysaccharidosis, muscular dystrophy, myeloproliferative disorders, myelodysplastic syndromes, multiple myeloma, multiple sclerosis, neuroblastoma, non-Hodgkin lymphoma, osteoarthritis, Parkinson's disease, paroxysmal nocturnal hemoglobinuria, pure red cell aplasia, rheumatoid arthritis, scleroderma, sexual dysfunction, severe combined immunodeficiency, sickle cell anemia, spinal cord injuries, stroke, systemic lupus erythrematosus, systemic sclerosis, thalassemia major, and Wiskott-Aldrich syndrome.

In some embodiments, contacting cells with a compound of Formula (I), (I-A), (I-B), (I-C), or (I-D), or a pharmaceutically acceptable salt thereof, can result in increasing or expanding the number of cells by from about 10 to about 50,000 fold. In some embodiments, a compound of Formula (I), (I-A), (I-B), (I-C), or (I-D), or a pharmaceutically acceptable salt thereof, described herein can increase or expand the number of cells by 1.05, 1.1, 1.2, 1.3, 1.4, 1.5, 2, 3, 5, 7, 10, 15, 20, 25, 50, 75, 100, 200, 300, 400, 500, 600, 700, 800, 900, 1,000, 2,000, 3,000, 4,000, 5,000, 6,000, 7,000, 8,000, 9,000, 10,000, 15,000, 20,000, 25,000, 30,000, 35,000, 40,000, 45,000, or 50,000-fold, or within a range defined by any two of the aforementioned values. In some embodiments, the cells may be stem cells, or progenitor cells. In some embodiments, the stem cells may be hematopoietic stem cells.

In some embodiments, contacting cells with a compound of Formula (I), is followed by, or carried out contemporaneously with, contacting cells with conditions, e.g., cytokines, etc., that promote differentiation into a desired differentiated cell population to produce an expanded differentiated cell population. In some embodiments, the differentiated cell population is a hematopoietic cell population.

In some embodiments, the increase in the number of hematopoietic stem cells may be determined by counting the number of CD34+ cells in a cell population treated with a compound of Formula (I), (I-A), (I-B), (I-C), or (I-D). For example, an increase in the number of CD34+ cells by 1.05, 1.1, 1.2, 1.3, 1.4, 1.5, 2, 3, 5, or 10-fold, or greater as compared to number of CD34+ cells in a population without expansion may be indicative of hematopoietic stem cell expansion.

In some embodiments, the increase in the number of hematopoietic stem cells may be determined by counting the number of CD34+ cells in a cell population treated with a compound of Formula (I), (I-A), (I-B), (I-C), or (I-D). For example, an increase in the number of CD34+ cells by 1.05, 1.1, 1.2, 1.3, 1.4, 1.5, 2, 3, 5, or 10-fold, or greater as compared to number of CD34+ cells in a population without expansion may be indicative of hematopoietic stem cell expansion.

Upon expanding a particular cell population, the cells may harvested. Harvesting is performed by separating the cell culture from the growing medium. Several techniques may be used to harvest the cells. For example, harvesting may be accomplished using methods including, but not limited to, centrifugation, microfiltration, depth filtration, tangential-flow filtration, filtration through absolute pore size membranes, or any combination thereof. Once harvested, the cells may be further expanded or frozen for later use.

In some embodiments, the population of cells can be stem cells, for example hematopoietic stem cells. In other embodiments, the population of cells can be progenitor cells. In other embodiments, the population of cells can be differentiated hematopoietic cells. In some embodiments, the population of cells can be derived from bone marrow. In other embodiments, the population of cells can be derived from umbilical cord blood. In other embodiments, the population of cells can be derived from placenta or placental perfusate. In some embodiments, the population of cells can be derived from peripheral blood.

Culturing cells is a method by which cell populations are grown under controlled conditions. In general, cell culture is performed in vitro. The culturing of cells (e.g., stem cells, hematopoietic stem cells, or progenitor cells) may be performed under conditions known to the person skilled in the art. For example, the CO₂ and O₂ content, nutritive media, duration, etc. can be determined by a person skilled in the art, and varies depending upon the starting cell population. In some embodiments, the culturing conditions may comprise the use of various cytokines and/or growth factors which include, but are not limited to, G-CSF, GM-CSF, SCF, FLT3-L, thrombopoietin, erythropoietin, IL-1, IL-3, IL-6, IL-11, and combinations thereof. In some embodiments, the culture conditions may comprise cytokines and/or growth factors, as generally known in the art.

In some embodiments, the expansion of cells using a compound of Formula (I), (I-A), (I-B), (I-C), or (I-D), or a pharmaceutically acceptable salt thereof, may be carried out in a variety of growth media. A growth medium is a solid, liquid, or semi-solid designed to support the growth of cells. In some embodiments, the expansion of cells may be carried out in a basal medium. In general, a basal medium may comprise amino acids, carbon sources, vitamins, serum proteins, various salts, divalent cations (e.g., Ca²⁺, Mg^{2+,} Mn²⁺, Cu²⁺, Fe²⁺, Co²⁺, Zn²⁺), and buffers and any other element suitable for use in expansion of cells. Examples of basal medium appropriate for a method of expanding cell populations provided herein include, but are not limited to, StemSpan® H3000-Defined Medium, CellGro® SCGM, StemPro®-34 SFM, StemSpan® SFEM—Serum-Free Expansion Medium, Clonetics® Lymphocyte Growth Media-3 LGM-3®, and PluriSTEM® Human ES/iPS Medium.

In some embodiments, one or more compounds of Formula (I), (I-A), (I-B), (I-C), or (I-D), or pharmaceutically acceptable salts thereof may be added to a growth medium. In some embodiments, the one or more compounds of Formula (I), (I-A), (I-B), (I-C), or (I-D) may be present in the medium at a concentration of at least 1 pM, at least 5 pM, at least 10 pM, at least 20 pM, at least 50 pM, at least 100 pM, at least 200 pM, at least 300 pM, at least 400 pM, at least 500 pM, at least 600 pM, at least 700 pM, at least 800 pM, at least 900 pM, at least 1 nM, at least 5 nM, at least 10 nM, at least 20 nM, at least 50 nM, at least 100 nM, at least 200 nM, at least 300 nM, at least 400 nM, at least 500 nM, at least 600 nM, at least 700 nM, at least 800 nM, at least 900 nM, at least 1 μM, at least 5 μM, at least 10 μM, at least 20 μM, at least 50 μM, or within a range defined by any two of the aforementioned concentrations. In some embodiments, the one or more compounds of Formula (I), (I-A), (I-B), (I-C), or (I-D) may be present in the medium at a concentration of at most 1 pM, at most 5 pM, at most 10 pM, at most 20 pM, at most 50 pM, at most 100 pM, at most 200 pM, at most 300 pM, at most 400 pM, at most 500 pM, at most 600 pM, at most 700 pM, at most 800 pM, at most 900 pM, at most 1 nM, at most 5 nM, at most 10 nM, at most 20 nM, at most 50 nM, at most 100 nM, at most 200 nM, at most 300 nM, at most 400 nM, at most 500 nM, at most 600 nM, at most 700 nM, at most 800 nM, at most 900 nM, at most 1 μM, at most 5 μM, at most 10 μM, at most 20 μM, at most 50 μM, or within a range defined by any two of the aforementioned concentrations

In some embodiments, the population of cells selected for expansion may be subjected to enrichment. As used herein, a population of cells that is “enriched” in cells having or lacking a particular marker refers to cell populations wherein at least 50%, 55%, 60%, 65%, 70%, 75%, 80%, 85%, 90%, or more of the cells in the populations have or lack a particular marker. For example, cell populations enriched in CD34+ comprise at least 50% 55%, 60%, 65%, 70%, 75%, 80%, 85%, 90%, or more CD34+ cells. In some embodiments, the population of cells may be selected based on particular cellular markers. For example, the population of cells may be selected based on the presence or absence of one or more markers including, but not limited to, CD3, CD11a, CD16, CD34, CD56, CD90, CD94, and CD133. Isolating starting cell population based on specific cellular markers may be achieved by methods known to the person skilled in the art including but not limited to flow cytometry and affinity purification.

In some embodiments, the population selected for expansion may comprise at least 100,000 nucleated cells. In some embodiments, the population selected for expansion may comprise at least 1,000,000 nucleated cells. In some embodiments, the population selected for expansion may comprise at least 10,000,000 nucleated cells. In some embodiments, the population selected for expansion may comprise at least 100,000,000 nucleated cells. In some embodiments, the population selected for expansion may comprise at least 1,000,000,000 nucleated cells. In some embodiments, the population selected for expansion may comprise at least 10,000,000,000 nucleated cells. In some embodiments, the population of cells selected for expansion may comprise from 100,000 to 10,000,000,000 nucleated cells. In some embodiments the cell population selected is enriched in CD34+ cells. In other embodiments the cell population selected is enriched in CD56+ cells. In some embodiments, the cell population selected is enriched in CD16+ cells. In some embodiments, the cell population selected is enriched in CD11a+ cells. In some embodiments, the cell population selected is enriched in CD9430 cells. In some embodiments, the cell population selected is enriched in CD3- cells.

In some embodiments, the cell population selected for expansion may be used directly for expansion (for example, without being frozen or stored for use at a later date). In other embodiments, the cell population selected for expansion may be frozen and/or stored for use at a later date.

In some embodiments, starting cell population selected for expansion may be cultured in the presence of one or more compounds of Formula (I), (I-A), (I-B), (I-C), or (I-D), or pharmaceutically acceptable salts thereof, for from about 3 days to about 90 days. For example, the contacting can occur for about 3 days, 6 days, 9 days, 12 days, 15 days, 18 days, 21 days, 24 days, 27 days, 30 days, 33 days, 36 days, 39 days, 42 days, 45 days, 48 days, 51 days, 54 days, 57 days, 60 days, 63 days, 66 days, 69 days, 72 days, 75 days, 78 days, 81 days, 84 days, 87 days, or 90 days, or within any range defined by two of the aforementioned values. In some embodiments, the starting cell population selected for expansion may be cultured for from about 5 days to about 15 days. For example the culturing can occur for about 5 days, 6 days, 7 days, 8 days, 9 days, 11 days, 12 days, 13 days, 14 days, 15 days, or within any range defined by two of the aforementioned values. In some embodiments, the culturing can occur for more than 90 days. In some embodiments, the culturing can occur for from about 2 hours, 4 hours, 6 hours, 8 hours, 10 hours, 12 hours, 16 hours, 20 hours, 24 hours, 30 hours, 36 hours, 42 hours, 48 hours, 54 hours, 60 hours, 66 hours, 72 hours, or within any range defined by two of the aforementioned values.

In some embodiments, the starting cell population may be cultured in the presence of one or more compounds of Formula (I), (I-A), (I-B), (I-C), or (I-D), or pharmaceutically acceptable salts thereof, for a duration that provides adequate time to expand the cell population. For example, the starting cell population may be cultured for a time adequate to increase the starting cell population by about 1.1, 1.2, 1.3, 1.4, 1.5, 1.6, 1.7, 1.8, 1.9, 2.0, 3.0, 5.0, 10, 15, 20, 25, 50, 75, 100, 200, 300, 400, 500, 600, 700, 800, 900, 1,000, 2,000, 3,000, 4,000, 5,000, 6,000, 7,000, 8,000, 9,000, 10,000, 15,000, 20,000, 25,000, 30,000, 35,000, 40,000, 45,000, of 50,000-fold, or within a range defined by any two of the aforementioned values.

In some embodiments, the cell population obtained after an expansion method provided herein may be used without further purification. In other embodiments, the cell population obtained after an expansion method provided herein may be subject to purification.

In some embodiments provided herein is a method of treating a subject by resuspending cells obtained by the methods described herein and administering said cells to a subject in need of treatment. In some embodiments, such a cell population may be resuspended in a pharmaceutically acceptable medium suitable for administration to a subject.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject is human. In some specific embodiments, the subject may be a bone marrow donor. In some embodiments, the subject may be a recipient of a bone marrow transplant. In some embodiments, the subject may have received chemotherapy. In some embodiments, the subject may have received radiation therapy.

As used herein, the terms “treat,” “treating,” “treatment,” “therapeutic,” and “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the subject's overall feeling of well-being or appearance, and may positively affect one or more symptoms or aspects of the disease while having effects on other aspects of the disease or on unrelated systems that may be considered undesirable.

The terms “therapeutically effective amount” and “effective amount” are used to indicate an amount of an active compound, or pharmaceutical agent, that elicits the biological or medicinal response indicated. For example, a therapeutically effective amount of compound can be the amount needed to prevent, treat, alleviate or ameliorate one or more symptoms or conditions of disease or prolong the survival of the subject being treated This response may occur in a tissue, system, animal or human and includes alleviation of the signs or symptoms of the disease being treated. Determination of an effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

In some embodiments, a compound of Formula (I), (I-A), (I-B), (I-C), or (I-D), or a pharmaceutically acceptable salt thereof, or a pharmaceutical composition that includes a compound described herein, can be used in combination with one or more additional agent(s). In some embodiments, a compound of Formula (I), (I-A), (I-B), (I-C), or (I-D), or a pharmaceutically acceptable salt thereof, can be used in combination with one or more agents commonly used for culturing stem cells and/or progenitor cells. For example, the additional agent can be IL6, TPO, SCF, or Flt3-L, or a combination thereof.

In some embodiments, a compound of Formula (I), (I-A), (I-B), (I-C), or (I-D), or a pharmaceutically acceptable salt thereof, can be administered with one or more additional agent(s) together in a single pharmaceutical composition. In some embodiments, a compound of Formula (I), (I-A), (I-B), (I-C), or (I-D), or a pharmaceutically acceptable salt thereof, can be administered with one or more additional agent(s) as two or more separate pharmaceutical compositions. For example, a compound of Formula (I), (I-A), (I-B), (I-C), or (I-D), or a pharmaceutically acceptable salt thereof, can be administered in one pharmaceutical composition, and at least one of the additional agents can be administered in a second pharmaceutical composition. If there are at least two additional agents, one or more of the additional agents can be in a first pharmaceutical composition that includes a compound of Formula (I), (I-A), (I-B), (I-C), or (I-D), or a pharmaceutically acceptable salt thereof, and at least one of the other additional agent(s) can be in a second pharmaceutical composition.

The order of administration of a compound of Formula (I), (I-A), (I-B), (I-C), or (I-D), or a pharmaceutically acceptable salt thereof, with one or more additional agent(s) can vary. In some embodiments, a compound of Formula (I), (I-A), (I-B), (I-C), or (I-D), or a pharmaceutically acceptable salt thereof, can be administered prior to all additional agents. In other embodiments, a compound of Formula (I), (I-A), (I-B), (I-C), or (I-D), or a pharmaceutically acceptable salt thereof, can be administered prior to at least one additional agent. In still other embodiments, a compound of Formula (I), (I-A), (I-B), (I-C), or (I-D), or a pharmaceutically acceptable salt thereof, can be administered concomitantly with one or more additional agent(s). In yet still other embodiments, a compound of Formula (I), (I-A), (I-B), (I-C), or (I-D), or a pharmaceutically acceptable salt thereof, can be administered subsequent to the administration of at least one additional agent. In some embodiments, a compound of Formula (I), (I-A), (I-B), (I-C), or (I-D), or a pharmaceutically acceptable salt thereof, can be administered subsequent to the administration of all additional agents.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials and in vitro studies.

The dosage may range broadly, depending upon the desired effects and the therapeutic indication. Alternatively dosages may be based and calculated upon the surface area of the patient, as understood by those of skill in the art. Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the subject. In some embodiments, the compounds will be administered for a period of continuous therapy, for example for a week or more, or for months or years.

In instances where human dosages for compounds have been established for at least some condition, those same dosages may be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

Additional embodiments are disclosed in further detail in the following examples, which are not in any way intended to limit the scope of the claims.

The present invention will now be further described by the following examples.

### Example 1

### Synthesis of Example Compounds

The following compounds were selected for synthesis and/or further analysis:

### 4-(2-((2-(benzo[b]thiophen-3-yl)-6-(isopropylamino)pyrimidin-4-yl)amino)ethyl)phenol) (“CRL1”)

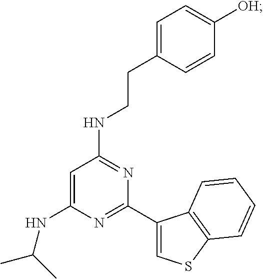 [SMILES: CC(C)Nc1cc(NCCc2ccc(O)cc2)nc(-c2csc3ccccc23)n1]

### 4-(2-((2-(benzo[b]thiophen-3-yl)-7-isopropylthieno[3,2-d]pyrimidin-4-yl)amino)ethyl)phenol)) (“CRL2”)

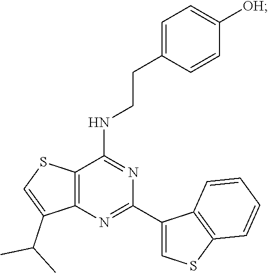 [SMILES: CC(C)c1csc2c(NCCc3ccc(O)cc3)nc(-c3csc4ccccc34)nc12]

### 4-(2-((2-(benzo[b]thiophen-3-yl)-7-isopropyl-6,7-dihydro-5H-pyrrolo[2,3-d]pyrimidin-4-yl)amino)ethyl)phenol (“CRL3”)

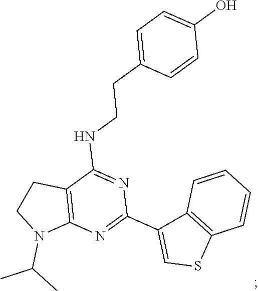 [SMILES: CC(C)N1CCc2c(NCCc3ccc(O)cc3)nc(-c3csc4ccccc34)nc21]

### 2-(benzo[b]thiophen-3-yl)-4-((4-hydroxyphenethyl)amino)-7-isopropyl-5,7-dihydro-6H-pyrrolo[2,3-d]pyrimidin-6-one (“CRL4”)

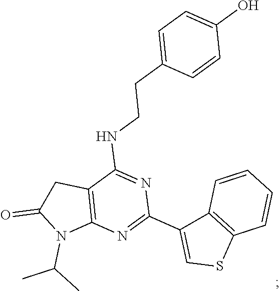 [SMILES: CC(C)N1C(=O)Cc2c(NCCc3ccc(O)cc3)nc(-c3csc4ccccc34)nc21]

### 3-((2-(benzo[b]thiophen-3-yl)-9-isopropyl-9H-purin-6-yl)oxy)propanamide (“CRL5”)

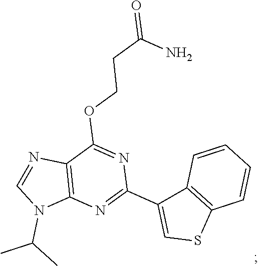 [SMILES: CC(C)n1cnc2c(OCCC(N)=O)nc(-c3csc4ccccc34)nc21]

### 4-(2-((2-(benzo[b]thiophen-3-yl)-8-(dimethylamino)pyrimido[5,4-d]pyrimidin-4-yl)amino)ethyl)phenol (“CRL6”)

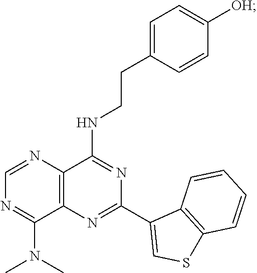 [SMILES: CN(C)c1ncnc2c(NCCc3ccc(O)cc3)nc(-c3csc4ccccc34)nc12]

### 5-(2-((2-(1H-indol-3-yl)ethyl)amino)-6-(sec-butylamino)pyrimidin-4-yl)nicotinonitrile (“CRL7”)

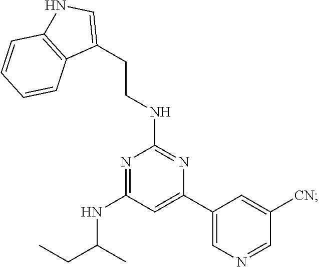 [SMILES: CCC(C)Nc1cc(-c2cncc(C#N)c2)nc(NCCc2c[nH]c3ccccc23)n1]

### N-(2-(1H-indol-3-yl)ethyl)-2-methyl-6-phenylthieno[2,3-d]pyrimidin-4-amine (“CRL8”)

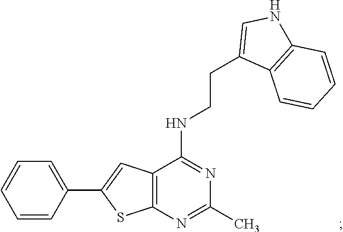 [SMILES: Cc1nc(NCCc2c[nH]c3ccccc23)c2cc(-c3ccccc3)sc2n1]

### N-(2-(1H-indol-3-yl)ethyl)-6-(4-fluorophenyl)thieno[2,3-d]pyrimidin-4-amine (“CRL9”)

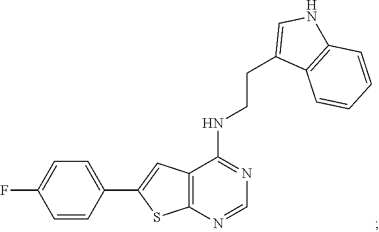 [SMILES: Fc1ccc(-c2cc3c(NCCc4c[nH]c5ccccc45)ncnc3s2)cc1]

### 3-(2-(benzo[b]thiophen-3-yl)-9-isopropyl-6-oxo-6,9-dihydro-1H-purin-1-yl)propanamide (“CRL10”)

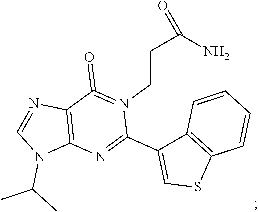

### N-(2-(1H-indol-3-yl)ethyl)-2-(5-fluoropyridin-3-yl)quinazolin-4-amine (“CRL11”)

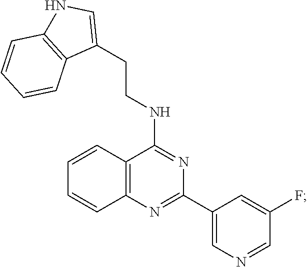 [SMILES: Fc1cncc(-c2nc(NCCc3c[nH]c4ccccc34)c3ccccc3n2)c1]

### 5-(4-((2-(1H-indol-3-yl)ethyl)amino)quinazolin-2-yl)nicotinonitrile (“CRL12”)

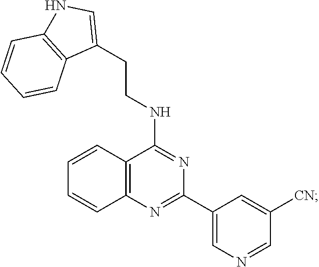 [SMILES: N#Cc1cncc(-c2nc(NCCc3c[nH]c4ccccc34)c3ccccc3n2)c1]

### N⁴-(2-(1H-indol-3-yl)ethyl)-N²-(sec-butyl)quinazoline-2,4-diamine (“CRL13”)

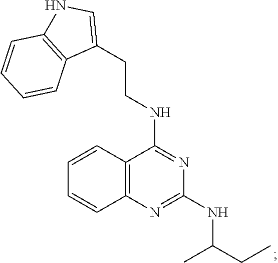 [SMILES: CCC(C)Nc1nc(NCCc2c[nH]c3ccccc23)c2ccccc2n1]

### 2-(benzo[b]thiophen-3-yl)-4-((4-hydroxyphenethyl)amino)-7-isopropyl-7H-pyrrolo[2,3-d]pyrimidine-5-carbonitrile (“CRL14”)

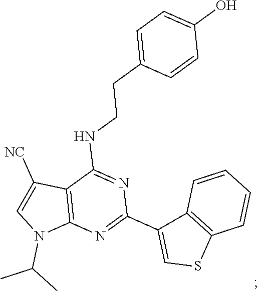 [SMILES: CC(C)n1cc(C#N)c2c(NCCc3ccc(O)cc3)nc(-c3csc4ccccc34)nc21]

### N-(2-(1H-indol-3-yl)ethyl)-6-(benzo[b]thiophen-3-yl)-3-isopropylimidazo[1,5-a]pyrazin-8-amine (“CRL15”)

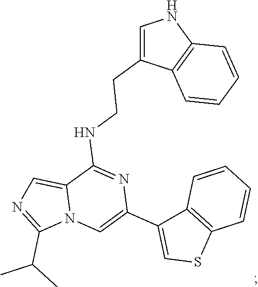 [SMILES: CC(C)c1ncc2c(NCCc3c[nH]c4ccccc34)nc(-c3csc4ccccc34)cn12]

### 4-(2-((6-(benzo[b]thiophen-3-yl)-3-isopropylimidazo[1,5-a]pyrazin-8-yl)amino)ethyl)phenol (“CRL16”)

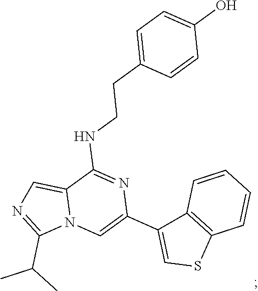 [SMILES: CC(C)c1ncc2c(NCCc3ccc(O)cc3)nc(-c3csc4ccccc34)cn12]

### 5-(4-((2-(1H-indol-3-yl)ethyl)amino)-7-isopropylthieno[3,2-d]pyrimidin-2-yl)nicotinonitrile (“CRL17”)

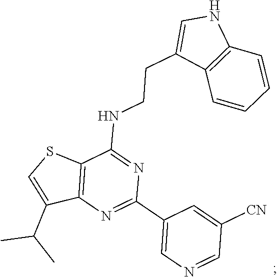 [SMILES: CC(C)c1csc2c(NCCc3c[nH]c4ccccc34)nc(-c3cncc(C#N)c3)nc12]

### N-(2-(1H-indol-3-yl)ethyl)-2-(5-fluoropyridin-3-yl)-7-isopropylthieno[3,2-d]pyrimidin-4-amine (“CRL18”)

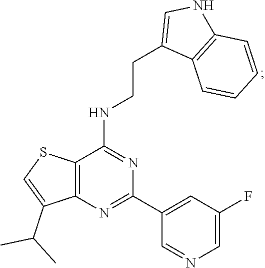 [SMILES: CC(C)c1csc2c(NCCc3c[nH]c4ccccc34)nc(-c3cncc(F)c3)nc12]

### N-(2-(1H-indol-3-yl)ethyl)-2-(5-fluoropyridin-3-yl)furo[3,2-d]pyrimidin-4-amine (“CRL19”)

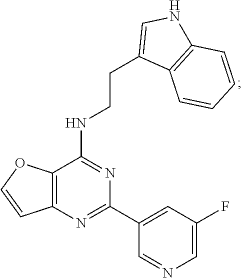 [SMILES: Fc1cncc(-c2nc(NCCc3c[nH]c4ccccc34)c3occc3n2)c1]

### N-(2-(1H-indol-3-yl)ethyl)-2-(5-methylpyridin-3-yl)furo[3,2-d]pyrimidin-4-amine (“CRL20”)

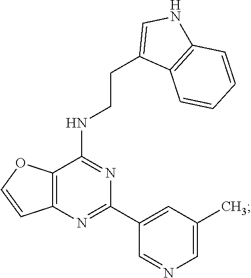 [SMILES: Cc1cncc(-c2nc(NCCc3c[nH]c4ccccc34)c3occc3n2)c1]

### N-(2-(1H-indol-3-yl)ethyl)-7-isopropyl-2-(5-methylpyridin-3-yl)thieno[3,2-d]pyrimidin-4-amine (“CRL21”)

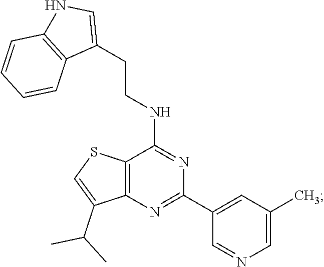 [SMILES: Cc1cncc(-c2nc(NCCc3c[nH]c4ccccc34)c3scc(C(C)C)c3n2)c1]

and

### 5-(4-((2-(1H-indol-3-yl)ethyl)amino)furo[3,2-d]pyrimidin-2-yl)nicotinonitrile (“CRL22”)

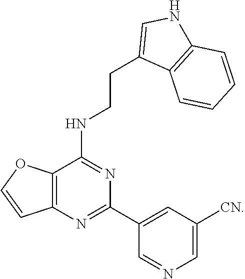 [SMILES: N#Cc1cncc(-c2nc(NCCc3c[nH]c4ccccc34)c3occc3n2)c1]

### PREPARATIONS OF INTERMEDIATES AND EXAMPLES

### General Experimental Details

Purification by chromatography refers to purification using a CombiFlash® Companion purification system or a Biotage SP1 purification system. Where products were purified using an Si cartridge, this refers to an Isolute® pre-packed polypropylene column containing unbounded activated silica with irregular particles with average size of 50 μm and nominal 60 Å porosity. Fractions containing the required product (identified by TLC and/or LCMS analysis) were pooled and concentrated in vacuo. Where HPLC was used for purification (purification by MDAP) fractions containing the required product (identified by TLC and/or LCMS analysis) were pooled and the solvent removed using a Biotage EV10 Evaporator. Alternatively the pooled product fraction was lyophilised.

NMR spectra were obtained on a Varian Unity Inova 400 spectrometer with a 5 mm inverse detection triple resonance probe operating at 400 MHz or on a Bruker Avance DRX 400 spectrometer with a 5 mm inverse detection triple resonance TXI probe operating at 400 MHz or on a Bruker Avance DPX 300 spectrometer with a standard 5 mm dual frequency probe operating at 300 MHz or on a Bruker Fourier 300 spectrometer with a 5 mm dual probe operating at 300 MHz. Shifts are given in ppm relative to tetramethyl silane.

TABLE 1
LCMS Method 1

| Instrumentation | Acquity UPLC (binary pump/PDA detector) + | | |
|---|---|---|---|
| | ZQ Mass Spectrometer | | |
| Column | ACQUITY UPLC BEH C₁₈ 1.7 μm, | | |
| | 100 × 2.1 mm, maintained at 40° C. | | |
| Mobile Phase A | 0.1% Aqueous formic acid (v/v) | | |
| Mobile Phase B | 0.1% Formic acid in acetonitrile (v/v) | | |
| Flow | 0.4 ml/min | | |
| Gradient Program | Time (mins) | % A | % B |
| | 0.0 | 95 | 05 |
| | 0.4 | 95 | 05 |
| | 6.0 | 05 | 95 |
| | 6.8 | 05 | 95 |
| | 7.0 | 95 | 05 |
| | 8.0 | 95 | 05 |

| | |
|---|---|
| Sample | 1 μl injection of a 0.2-0.5 mg/ml solution |
| | in an appropriate solvent at 20° C. |
| Detectors | UV, diode array 200-500 nm |
| | MS, mass 100-800 (or −1500 for HM method) |
| | in ES + & ES-(no split to MS) |
| Data Analysis | Peak area percentage (APCT) with an |
| | integration threshold of 0.2% (relative) |

TABLE 2
LCMS Method 2

| Instrumentation | Acquity i-Class (quarternary pump/PDA detector) + | | |
|---|---|---|---|
| | Quattro Micro Mass Spectrometer | | |
| Column | ACQUITY UPLC BEH C₁₈ 1.7 μm, | | |
| | 100 × 2.1 mm, maintained at 40° C. | | |
| Mobile Phase A | 0.1% Aqueous formic acid (v/v) | | |
| Mobile Phase B | 0.1% Formic acid in acetonitrile (v/v) | | |
| Flow | 0.4 ml/min | | |
| Gradient Program | Time (mins) | % A | % B |
| | 0.0 | 95 | 05 |
| | 0.4 | 95 | 05 |
| | 6.0 | 05 | 95 |
| | 6.8 | 05 | 95 |
| | 7.0 | 95 | 05 |
| | 8.0 | 95 | 05 |

| | |
|---|---|
| Sample | 1 μl injection of a 0.5 mg/ml solution |
| | in an appropriate solvent at 20° C. |
| Detectors | UV, diode array 200-500 nm |
| | MS, mass 100-800 (or −1500 for HM method) |
| | in ES + & ES-(no split to MS) |
| Data Analysis | Peak area percentage (APCT) with an |
| | integration threshold of 0.2% (relative) |

TABLE 3
LCMS Method 3

| Instrumentation | Acquity H-Class (quaternary pump/PDA detector) + | | |
|---|---|---|---|
| | QDa Mass Spectrometer | | |
| Column | Acquity UPLC CSH C18 1.7 μm, | | |
| | 50 × 2.1 mm at 40° C. | | |
| Mobile Phase A | 0.1% Aqueous formic acid (v/v) | | |
| Mobile Phase B | 0.1% Formic acid in acetonitrile (v/v) | | |
| Flow | 1.0 ml/min | | |
| Gradient Program | Time (mins) | % A | % B |
| | 0.0 | 97 | 03 |
| | 4 | 01 | 99 |
| | 4.4 | 01 | 99 |
| | 4.5 | 97 | 03 |
| | 5 | 97 | 03 |

| | |
|---|---|
| Sample | 1 μl injection (Open Access) |
| Detectors | UV, diode array 190-450 nm |
| | MS, mass 160-1000 (or 100-800 for LM |
| | or 160-1250 for HM method) in ES + & ES- |

### MDAP Method (Acidic)

Agilent Technologies 1260 Infinity purification system with an XSELECT CSH Prep C₁₈ column (19×250 mm, 5 μm OBD) maintained at RT
Mobile Phase A: 0.1% aqueous formic acid
Mobile Phase B: 0.1% formic acid in acetonitrile
Flow Rate: 20 ml/min
Gradient Program: 10%-95%, 22 min, centered around a specific focused gradient
Sample: Injection of a 20-60 mg/ml solution in DMSO (+optional formic acid and water)

### MDAP Method (Basic)

Agilent Technologies 1260 Infinity purification system with an XBridge Prep C18 OBD column (19×250 mm, 5 μm OBD) maintained at RT
Mobile Phase A: 0.1% aqueous ammonia
Mobile Phase B: 0.1% ammonia in acetonitrile
Flow Rate: 20 ml/min
Gradient Program: 10%-95%, 22 min, centered around a specific focused gradient
Sample: Injection of a 20-60 mg/ml solution in DMSO+optional formic acid and water)

### Preparative HPLC Method (Acidic Conditions)

Where compounds were purified by HPLC they were carried out on a C18-reverse-phase column (250×21.2 mm Phenomenex Kinetex with 5 μm particle size). Specific eluting mixtures are described and, unless otherwise stated, peaks were detected by UV (254 nm). Fractions containing the pure product were generally combined and freeze-dried to give a solid.

### Preparative HPLC Method (Basic Conditions)

Where compounds were purified by HPLC they were carried out on a C18-reverse-phase column (250×21.2 mm Phenomenex Kinetex EVO with 5 μm particle size). Specific eluting mixtures are described and, unless otherwise stated, peaks were detected by UV (254 nm). Fractions containing the pure product were generally combined and freeze-dried to give a solid.

### Abbreviations Used in the Experimental Section:

CH₃CN Acetonitrile
DCM Dichloromethane
DIPEA Di-isopropylethylamine
DMF N,N-Dimethylformamide
DMSO Dimethylsulphoxide
h Hours
HCl Hydrochloric acid
HCO₂H Formic acid
H₂O Water
HPLC High performance liquid chromatography
H₂SO₄ Sulfuric acid
IMS Industrial methylated spirits
IPA Isopropyl alcohol
LCMS Liquid chromatography-mass spectrometry
LiAlH₄ Lithium aluminum hydride
LDA Lithium diisopropylamide
MDAP Mass-directed autopurification
MgSO₄ Magnesium sulfate
MW Microwave
NaHCO₃ Sodium bicarbonate
NaOH Sodium hydroxide
Na₂SO₄ Sodium sulfate
NH₄OH Ammonium hydroxide
NMR Nuclear Magnetic Resonance
POCl₃ Phosphorus (V) oxychloride
Rt Retention time
TEA Triethylamine
THF Tetrahydrofuran

### Compound Example 1: 3-{[2-(benzo[b]thiophen-3-yl)-9-isopropyl-9H-purin-6-yl]oxy}propanamide and 2: 3-[-2-(Benzo[b]thiophen-3-yl)-9-isopropyl-6-oxo-6,9-dihydro-1H-purin-1-yl]propanamide

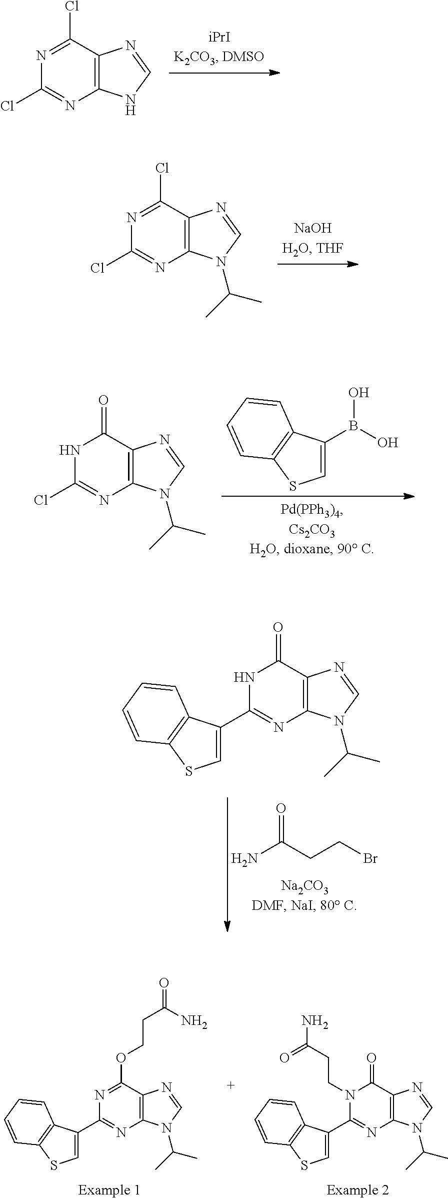

### Intermediate 1: 2,6-Dichloro-9-isopropyl-9H-purine

2-Iodopropane (7.9 mL) was added to a stirred, ice-cooled suspension of 2,6-dichloro-9H-purine (3.0 g) and potassium carbonate (6.58 g) in DMSO (15 mL) then the mixture was allowed to warm to room temperature and stirred overnight. The mixture was diluted with ethyl acetate and water and the layers were separated. The aqueous layer was further extracted with ethyl acetate and the combined organic layers were washed with water and brine, then dried (Na₂SO₄) and filtered. The filtrate was concentrated in vacuo and the residue was purified by chromatography on silica eluting with 0-20% acetone in DCM to give the title compound as a white solid (2.53 g). ¹H NMR (400 MHz, CDCl₃) δ 8.17 (1H, s), 4.96-4.88 (1H, m), 1.66 (6H, d, J=6.8 Hz); LCMS (Method 3) Rt 1.11 min m/z 231/233/235 [MH⁺].

### Intermediate 2: 2-Chloro-9-isopropyl-6H-purine-6-one

Aqueous sodium hydroxide (1 M, 35 mL) was added to a solution of Intermediate 1 (2.53 g) in THF (35 mL) and the resultant mixture was stirred at room temperature overnight. Further aqueous sodium hydroxide (1 M, 35 mL) was added and the mixture was stirred for a further 7 hours. The mixture was treated with acetic acid (3.1 mL) to take the pH to 4 then diluted with ethyl acetate. The layers were separated and the organic layer was washed with water, and brine then dried (Na₂SO₄) and filtered. The filtrate was concentrated in vacuo and the residue was combined with an earlier experiment using 0.49 g of Intermediate 1 and purified by chromatography on silica eluting with 0-5% methanol in DCM to give the title compound as an off-white solid (0.54 g). ¹H NMR (400 MHz, CDCl₃) 12.48 (br s, 1H), 7.89 (s, 1H), 4.80 (sept, J=6.8 Hz, 1H), 1.62 (d, J=6.8 Hz, 6H); LCMS (Method 3) Rt 0.79 min m/z 213/215 [M+H]⁺.

### Intermediate 3: 2-(Benzo[b]thiophen-3-yl)-9-isopropyl-1,9-dihydro-6H-purine-6-one

A mixture of Intermediate 2 (0.433 g), benzo[b]thien-3-yl boronic acid (0.544 g) and cesium carbonate (2 M aqueous solution, 2 mL) in dioxane (10 mL) was degassed under a stream of argon. Tetrakis-(triphenylphosphine)palladium (0) (0.235 g) was added and the vial was sealed and heated under argon for 4 hours. After cooling, the mixture was concentrated in vacuo and the residue was partitioned between DCM and water. The aqueous layer was further extracted with DCM and the combined organic layers were washed with water and brine then filtered through a phase separator. The filtrate was concentrated in vacuo and the residue was triturated with a mixture of methanol and DCM to give the title compound as a white solid (0.24 g). A further 0.05 g product was isolated from the filtrate by concentration and chromatographic purification of the residue using silica and eluting with 0-5% methanol in DCM. ¹H NMR (300 MHz, d6-DMSO) δ 12.48 (1H, s), 8.80 (1H, d, J=8.5 Hz), 8.71 (1H, s), 8.24 (1H, s), 8.13 (1H, d, J=7.9 Hz), 7.58-7.46 (2H, m), 4.89-4.80 (1H, m), 1.60 (6H, d, J=6.7 Hz); LCMS (Method 3) Rt 1.44 min m/z 311 [M+H]⁺.

### Compound Example 1: 3-{[2-(Benzo[b]thiophen-3-yl)-9-isopropyl-9H-purin-6-yl]oxy}propanamide

A mixture of Intermediate 3 (0.15 g) sodium carbonate (0.205 g), 3-bromopropanamide (0.147 g) and sodium iodide (0.0036 g) in dry DMF (5 mL) was stirred and heated at 80° C. under argon for 4 hours. Further sodium carbonate (0.201 g) and 2-bromopropanamide (0.15 g) were added and heating was continued at 80° C. for an additional 1.5 hours. After cooling, the mixture was poured into water and the precipitated solid was collected by filtration then redissolved in a mixture of DCM and methanol, dried (Na₂SO₄) and filtered. The residue was triturated with diethyl ether and the solid was collected by filtration to give the title compound as a white solid (0.122 g). ¹H NMR (400 MHz, d6-DMSO) δ 9.17 (1H, d, J=7.9 Hz), 8.77 (1H, s), 8.49 (1H, s), 8.10 (1H, d, J=7.9 Hz), 7.58-7.44 (3H, m), 6.97 (1H, s), 4.99-4.87 (3H, m), 2.72 (2H, t, J=6.2 Hz), 1.65 (6H, d, J=6.7 Hz); LCMS (Method 1) Rt 4.48 min m/z 382 [M+H]⁺.

### Compound Example 2: 3-[2-(Benzo[b]thiophen-3-yl)-9-isopropyl-6-oxo-6,9-dihydro-/H-purin-1-yl]propanamide

The diethyl ether filtrate from the formation of Example 1 was concentrated in vacuo and the residue was purified by MDAP (basic method) to give a white solid (0.006 g). ¹H NMR (400 MHz, d6-DMSO) δ 8.28 (1H, s), 8.22 (1H, s), 8.15-8.12 (1H, m), 7.69-7.66 (1H, m), 7.50-7.41 (2H, m), 7.26 (1H, s), 6.74 (1H, s), 4.72-4.64 (1H, m), 4.08-4.01 (2H, m), 2.42-2.36 (2H, m), 1.49 (6H, d, J=6.8 Hz); LCMS (Method 1) Rt 3.45 min m/z 382 [M+H]⁺.

### Compound Example 3: N-[2-(1H-Indol-3-yl)ethyl]-2-(5-fluoropyridin-3-yl)quinazolin-4-amine

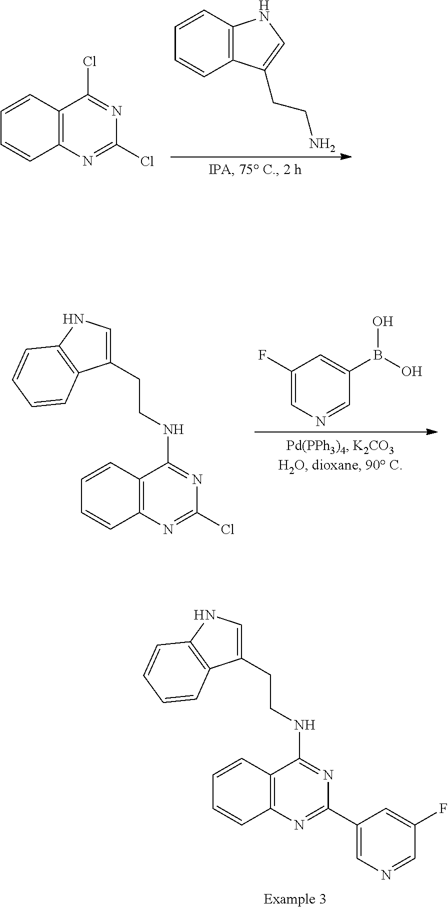 [SMILES: Clc1nc(Cl)c2ccccc2n1 ; NCCc1c[nH]c2ccccc12 ; Clc1nc(NCCc2c[nH]c3ccccc23)c2ccccc2n1 ; OB(O)c1cncc(F)c1 ; Fc1cncc(-c2nc(NCCc3c[nH]c4ccccc34)c3ccccc3n2)c1]

### Intermediate 4: N-[2-(1H-Indol-3-yl)ethyl]-2-chloroquinazolin-4-amine

A mixture of 2,4-dichloroquinazoline (0.3 g) and tryptamine (0.266 g) in IPA (10 mL) was sealed in a microwave vial and then heated at 75° C. for 2 hours. After cooling, the mixture was partitioned between water and ethyl acetate and the layers were separated. The organic layer was washed with water and brine, dried (Na₂SO₄) and filtered. The filtrate was concentrated in vacuo and the residue was purified by chromatography on silica eluting with 0-50% ethyl acetate in iso-hexane to give the title compound as a yellow foam (0.3 g). ¹H NMR (300 MHz, d6-DMSO) δ 10.85 (1H, s), 8.96-8.90 (1H, m), 8.26-8.23 (1H, m), 7.80 (1H, ddd, J=1.3, 7.0, 8.3 Hz), 7.71 (1H, d, J=7.8 Hz), 7.63 (1H, dd, J=0.8, 8.6 Hz), 7.54 (1H, ddd, J=1.3, 7.0, 8.2 Hz), 7.37-7.33 (1H, m), 7.21 (1H, d, J=2.2 Hz), 7.08 (1H, ddd, J=1.1, 7.0, 8.1 Hz), 7.02-6.95 (1H, m), 3.83-3.74 (2H, m), 3.11-3.03 (2H, m); LCMS (Method 3) Rt 1.38 min m/z 323/325 [M+H]⁺.

### Compound Example 3: N-[2-(1H-Indol-3-yl)ethyl]-2-(5-fluoropyridin-3-yl)quinazolin-4-amine

A mixture of Intermediate 4 (0.1 g), 5-fluoropyridin-3-ylboronic acid (0.061 g) and aqueous potassium carbonate solution (2 M, 0.31 mL) in dioxane (2 mL) was degassed under argon then tetrakis(triphenylphosphine)palladium (0) (0.036 g) was added. The mixture was then heated at 90° C. in a microwave for 30 minutes. After cooling, the mixture was poured into water and extracted with ethyl acetate, washed with water and brine, dried (Na₂SO₄) and filtered. The filtrate was concentrated in vacuo and the residue was purified by MDAP (basic method) to give the title compound as a white solid (0.072 g). ¹H NMR (400 MHz, DMSO) δ 10.84 (1H, s), 9.50 (1H, t, J=1.6 Hz), 8.72-8.68 (2H, m), 8.52-8.47 (1H, m), 8.28 (1H, d, J=8.3 Hz), 7.83-7.81 (2H, m), 7.66-7.63 (1H, m), 7.58-7.52 (1H, m), 7.36-7.33 (1H, m), 7.24 (1H, d, J=2.2 Hz), 7.10-7.05 (1H, m), 7.00-6.95 (1H, m), 4.01-3.94 (2H, m), 3.19-3.13 (2H, m); LCMS (Method 1) Rt 3.78 m/z 384 [M+H]⁺.

### Compound Example 4: 5-{4-[(2-{1H-Indol-3-yl}ethyl)amino]quinazolin-2-yl }nicotinonitrile

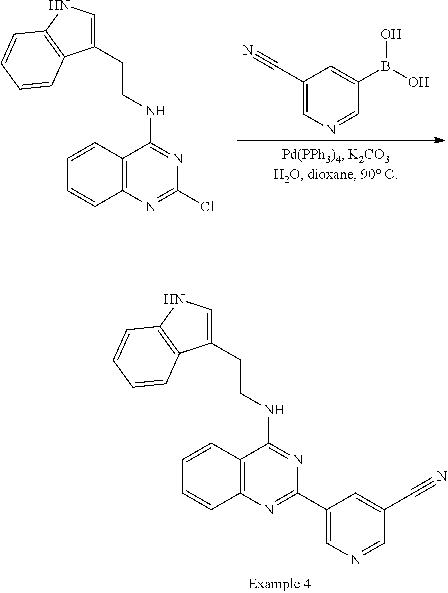 [SMILES: Clc1nc(NCCc2c[nH]c3ccccc23)c2ccccc2n1 ; N#Cc1cncc(B(O)O)c1 ; N#Cc1cncc(-c2nc(NCCc3c[nH]c4ccccc34)c3ccccc3n2)c1]

Prepared in a similar manner to Example 3, starting from Intermediate 4 (0.1 g) and 5-cyanopyridin-3-ylboronic acid (0.064 g) to give the title compound as a white solid (0.038 g). ¹H NMR (400 MHz, d6-DMSO) δ 10.83 (1H, s), 9.82-9.81 (1H, m), 9.14 (1H, d, J=2.0 Hz), 9.05 (1H, t, J=2.0 Hz), 8.73 (1H, t, J=5.6 Hz), 8.31-8.27 (1H, m), 7.83-7.82 (2H, m), 7.65 (1H, d, J=7.8 Hz), 7.59-7.54 (1H, m), 7.35-7.32 (1H, m), 7.23 (1H, d, J=2.3 Hz), 7.09-6.98 (2H, m), 4.03-3.95 (2H, m), 3.16 (2H, t, J=6.9 Hz); LCMS (Method 1) Rt 4.09 min m/z 391 [M+H]⁺.

### Compound Example 5: N⁴-[2-(1H-indol-3-yl)ethyl]-N²-sec-butyl)quinazolin-2,4-diamine

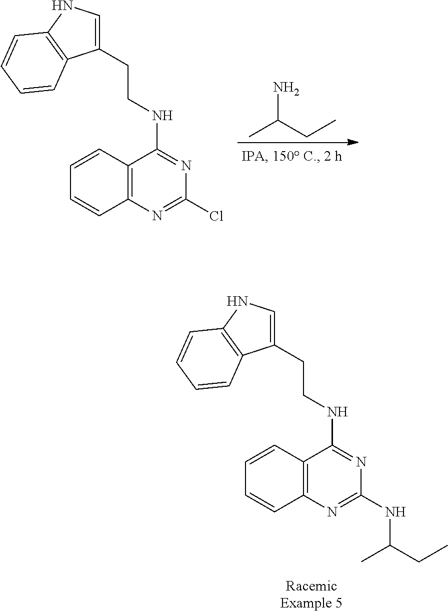

A mixture of Intermediate 4 (0.1 g) and sec-butylamine (0.094 mL) in isopropanol (2 mL) was heated in the microwave at 120° C. for 30 minutes. Further sec-butylamine (0.4 mL) was added and the mixture was heated in the microwave at 150° C. for a total of 1.25 hours. After cooling, the mixture was concentrated in vacuo and the residue was purified by MDAP (basic method) to give the title compound as a white solid (0.046 g). ¹H NMR (400 MHz, d6-DMSO) δ 10.82 (1H, s), 7.94-7.91 (2H, m), 7.61-7.58 (1H, m), 7.47-7.42 (1H, m), 7.36-7.33 (1H, m), 7.22-7.17 (2H, m), 7.07 (1H, ddd, J=1.1, 7.0, 8.2 Hz), 7.01-6.95 (2H, m), 6.19 (1H, s), 4.10-4.01 (1H, m), 3.76 (2H, dd, J=6.4, 14.4 Hz), 3.06 (2H, t, J=7.6 Hz), 1.61-1.40 (2H, m), 1.13 (3H, d, J=6.5 Hz), 0.88 (3H, t, J=7.4 Hz); LCMS (Method 1) Rt 3.73 m/z 360 [M+H]⁺.

### Compound Example 6: 2-(Benzo[b]thiophen-3-yl)-4-[(4-hydroxyphenethyl)amino]-7-isopropyl-7H-pyrrolo[2,3-d]pyrimidine-5-carbonitrile

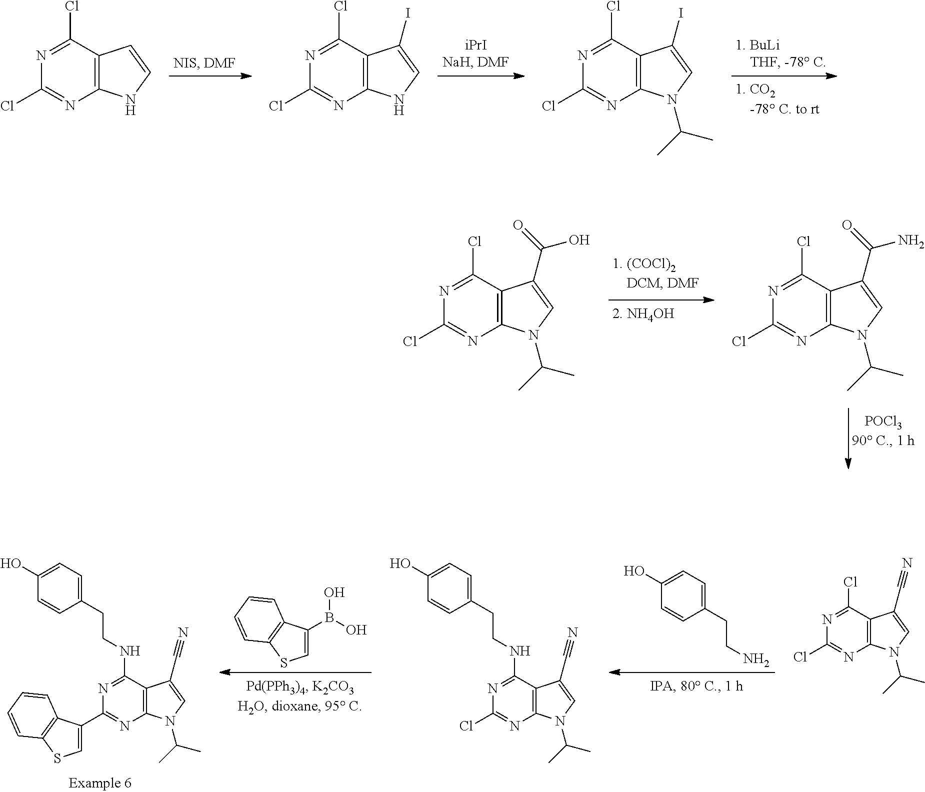

### Intermediate 5: 2,4-Dichloro-5-iodo-7H-pyrrolo[2,3-d]pyrimidine

N-iodosuccinimide (3.14 g) was added in portions to a solution of 2,4-dichloro-7H-pyrrolo[2,3-d]pyrimidine (2.5 g) in DMF (13.5 mL) warming to 40° C. to ensure initiation. On completion of the addition the mixture was allowed to cool to room temperature and stirred for 2 hours. The mixture was poured into ice/water with stirring and the resultant precipitate was collected by filtration and washed with cold water and dried in vacuo to give the title compound as a pale pink solid (3.93 g). NMR (400 MHz, d6-DMSO) δ 13.13 (1H, s), 7.98 (1H, s); LCMS (Method 3) Rt 1.24 min m/z 313/315/317 [M+H]⁺.

### Intermediate 6: 2,4-Dichloro-5-iodo-7-isopropyl-7H-pyrrolo[2,3-d]pyrimidine

Prepared in a similar manner to Intermediate 1, starting from Intermediate 5 (1 g), 2-iodopropane (0.95 mL) and sodium hydride (60%, 0.255 g) in DMF (10 mL) to give the title compound as a white solid (0.65 g). ¹H NMR (300 MHz, CDCl₃) δ 7.44 (1H, s), 5.15-5.05 (1H, m), 1.52 (6H, t, J=6.2 Hz); LCMS (Method 3) Rt 1.70 min m/z 356/358 [M+H]⁺.

### Intermediate 7: 2,4-Dichloro-7-isopropyl-7H-pyrrolo[2,3-d]pyrimidine-5-carboxylic acid

A solution of Intermediate 6 (0.65 g) in dry THF (2 mL) was added to a stirred, cooled solution of n-butyllithium (1.6 M in hexanes) in dry THF (10 mL) while maintaining the temperature below −70° C. The resultant mixture was stirred at −78° C. for 15 minutes then carbo dioxide gas was bubbled through the solution while allowing the temperature to rise to room temperature. Acetic acid (0.3 mL) was added, followed by water (50 mL) and the mixture was extracted with ethyl acetate. The combined organic layers were washed with water and brine, dried (Na₂SO₄) and filtered. The filtrate was concentrated in vacuo and the residue was stirred in iso-hexane overnight. The solid was collected by filtration and air dried to give the title compound as a white solid (0.174 g). ¹H NMR (300 MHz, d6-DMSO) δ 12.73 (1H, s), 8.57 (1H, s), 5.05-4.94 (1H, m), 1.51-1.47 (6H, m); LCMS (Method 3) Rt 1.30 min m/z 274/276 [M+H]⁺.

### Intermediate 8: 2,4-Dichloro-7-isopropyl-7H-pyrrolo[2,3-d]pyrimidine-5-carboxamide

Oxalyl chloride (0.11 g) was added to a solution of Intermediate 7 (0.174 g) in DCM (10 mL) containing a few drops of DMF under argon. The resultant mixture was stirred for 45 minutes then concentrated in vacuo. The residue was redissolved in DCM (10 mL) under argon and aqueous ammonium chloride (35%, 3.0 mL) was added. The mixture was stirred for 2 hours then poured into water and extracted with DCM. The organic layer was washed with brine then filtered through a phase separator. The filtrate was concentrated in vacuo and the residue was purified by chromatography on silica eluting with 0-100% ethyl acetate in iso-hexane to give the title compound as a white solid (0.1 g). ¹H NMR (300 MHz, CDCl₃) δ 8.17 (1H, s), 5.20-5.10 (1H, m), 1.55 (6H, d, J=6.6 Hz); LCMS (Method 3) Rt 1.03 min m/z 273/275 [M+H]⁺.

### Intermediate 9: 2,4-Dichloro-7-isopropyl-7H-pyrrolo[2,3-d]pyrimidine-5-carbonitrile

A mixture of Intermediate 8 (0.1 g) in phosphorus oxychloride (3 mL) was stirred and heated at 90° C. for 1 hour. After cooling, the mixture was added cautiously to a stirred mixture of aqueous sodium bicarbonate and ethyl acetate. The layers were separated and the aqueous layer was further extracted with ethyl acetate. The combined organic layers were washed with water and brine, dried (Na₂SO₄) and filtered. The filtrate was concentrated in vacuo to give the title compound as a white solid (0.09 g). ¹H NMR (300 MHz, CDCl₃) δ 7.86 (1H, s), 5.20-5.05 (1H, m), 1.58 (6H, d, J=7.0 Hz); LCMS (Method 3) Rt 1.37 min m/z 255/257 [M+H]⁺.

### Intermediate 10: 2-Chloro-4-[(4-hydroxyphenethyl)amino]-7-isopropyl-7H-pyrrolo[2,3-d]pyrimidine-5-carbonitrile

A mixture of Intermediate 9 (0.09 g) and 4-(2-aminoethyl)phenol (0.058 g) in IPA (5 mL) was stirred and heated at 80° C. for 1 hour. After cooling, the mixture was concentrated in vacuo and the residue was purified by chromatography on silica, eluting with 0-100% ethyl acetate in iso-hexane to give the title compound as a white solid (0.06 g). ¹H NMR (300 MHz, d6-DMSO) δ 9.20 (1H, s), 8.38 (1H, s), 7.09-7.02 (2H, m), 6.72-6.66 (2H, m), 4.88-4.78 (1H, m), 3.68-3.59 (2H, m), 2.83-2.76 (2H, m), 1.43 (6H, d, J=6.7 Hz); LCMS (Method 3) Rt 1.39 min m/z 356/358 [M+H]⁺.

### Compound Example 6: 2-(Benzo[b]thiophen-3-yl)-4-[(4-hydroxyphenethyl)amino]-7-isopropyl-7H-pyrrolo[2,3-d]pyrimidine-5-carbonitrile

A mixture of Intermediate 10 (0.06 g), benzo[b]thien-3-ylboronic acid (0.042 g) and potassium carbonate (2 M aqueous solution, 0.17 mL) in dioxane (2 mL) was degassed under a stream of argon then tetrakis(triphenylphosphine)palladium (0) (0.19 g) was added and the resultant mixture was stirred and heated in the microwave at 95° C. for 45 minutes. After cooling, the mixture was poured into water and extracted with ethyl acetate, washed with water and brine, dried (Na₂SO₄) and filtered. The filtrate was concentrated in vacuo and the residue was purified by MDAP (basic method) to give the title compound as a pale yellow solid (0.029 g). ¹H NMR (400 MHz, d6-DMSO) δ 9.22-9.16 (2H, m), 8.68 (1H, s), 8.39 (1H, s), 8.08-8.06 (1H, m), 7.51-7.42 (2H, m), 7.16-7.10 (2H, m), 6.74-6.69 (2H, m), 6.69-6.63 (1H, m), 5.15-5.04 (1H, m), 3.89-3.81 (2H, m), 2.91 (2H, t, J=7.5 Hz), 1.55 (6H, d, J=6.7 Hz); LCMS (Method 2) Rt 6.05 min m/z 454 [M+H]⁺.

### Compound Example 7: 4-(2-{[2-(Benzo[b]thiophen-3-yl)-7-isopropylthieno[3,2-d]pyrimidin-4-yl]amino}ethyl)phenol

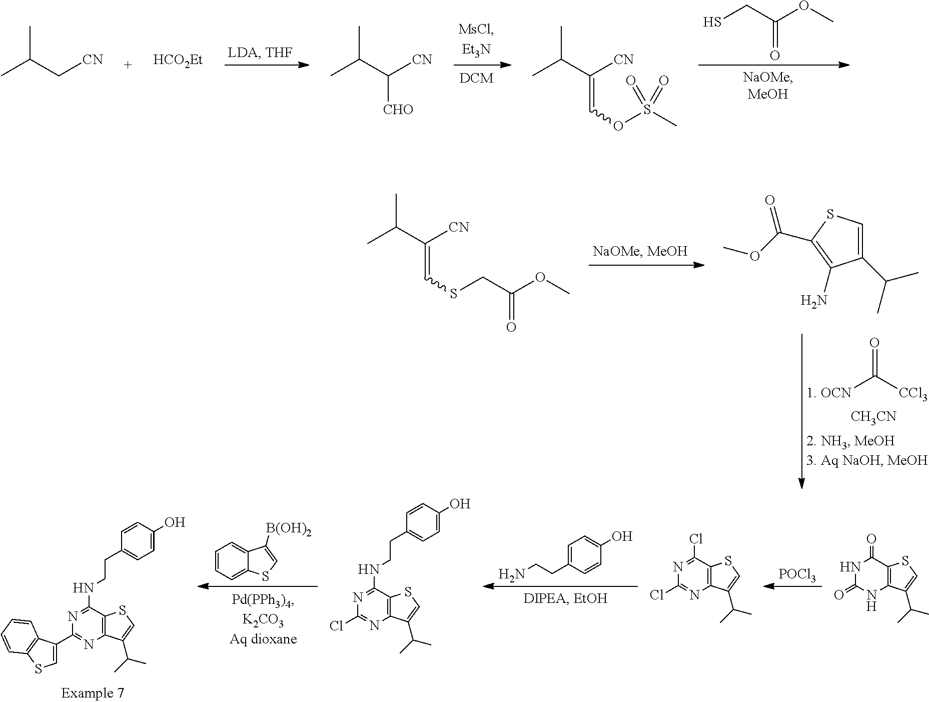

### Intermediate 11: 2-Formyl-3-methylbutanenitrile

Isovalerylnitrile (10 g) was added to a stirred, cooled solution of LDA (2.0 M in THF, 60.2 mL) in THF (40 mL) while maintaining the temperature below −70° C. On completion of the addition, the mixture was stirred for 10 minutes at −78° C. The resultant solution was added by cannula to a solution of ethyl formate (10.2 mL) in THF (50 mL) while maintaining the temperature below −70° C. The mixture was then stirred overnight while allowing the temperature to rise slowly to room temperature. The mixture was acidified to pH₃ by addition of 1 M hydrochloric acid. The layers were separated and the aqueous layer was extracted with ethyl acetate. The combined organic layers were dried (MgSO₄) and filtered. The filtrate was concentrated in vacuo and the residue was purified by chromatography on silica eluting with 0-100% ethyl acetate in cyclohexane to give the title compound as an orange oil (11.4 g). ¹H NMR (300 MHz, CDCl₃) δ 9.58 (1H, d, J=1.1 Hz), 3.42 (1H, dd, J=1.1, 4.8 Hz), 2.53-2.42 (1H, m), 1.20 (3H, d, J=6.5 Hz), 1.12 (3H, d, J=6.9 Hz).

### Intermediate 12: 2-Cyano-3-methylbut-1-en-1-yl methanesulfonate

A solution of methanesulfonyl chloride (11.4 g) in DCM (10 mL) was added dropwise to a stirred, cooled solution of Intermediate 11 (10.0 g) and trimethylamine (27.3 g) in DCM (190 mL) while maintaining the temperature below 5° C. The cooling bath was removed and the mixture was stirred while the temperature rose to room temperature. The mixture was partitioned between water and DCM and the layers were separated. The aqueous layer was further extracted with DCM and the combined organic layers were dried (MgSO₄) and filtered. The filtrate was concentrated in vacuo and the residue was purified by chromatography on silica, eluting with 0-3% methanol in DCM to give the title compound as a yellow oil which is a 3:2 mixture of cis and trans isomers (4.81 g). ¹H NMR (300 MHz, CDCl₃) δ 7.19 (0.4H, s), 7.12 (0.6H, s), 3.22 (1.8H, s), 3.20 (1.2H, s), 3.03-2.95 (0.4H, m), 2.61-2.49 (0.6H, m), 1.20 (3.6H, d, J=6.8 Hz), 1.16 (2.4H, d, J=6.8 Hz).

### Intermediate 13: Methyl 2-[(2-cyano-3-methylbut-1-en-1-yl)thio]acetate

Sodium methoxide (1.25 g) was added to a solution of Intermediate 12 (4.8 g) and methyl 2-mercaptoacetate (2.79 g) in methanol (60 mL) and the resultant mixture was stirred and heated at reflux for 5 hours. After cooling, the mixture was concentrated in vacuo and the residue was partitioned between water and ethyl acetate. The layers were separated and the aqueous layer was further extracted with ethyl acetate. The combined organic layers were dried (MgSO₄) and filtered. The filtrate was concentrated in vacuo and the residue was purified by chromatography on silica eluting with 0-50% ethyl acetate in iso-hexane to give the title compound (2.32 g). ¹H NMR (300 MHz, CDCl₃) δ 6.88 (1H, d, J=0.8 Hz), 3.78 (3H, s), 3.49 (2H, s), 2.60-2.51 (1H, m), 1.16 (6H, d, J=6.8 Hz); LCMS (Method 3) Rt 1.17 min m/z 200 [M+H]⁺.

### Intermediate 14: Methyl 3-amino-4-isopropylthiophene-2-carboxylate

A mixture of Intermediate 13 (2.08 g) and sodium methoxide (0.677 g) in methanol (16 mL) was heated in the microwave at 100° C. for 1 hour. After cooling, the mixture was partitioned between water and ethyl acetate. The layers were separated and the aqueous layer was further extracted with ethyl acetate. The combined organic layers were dried (MgSO₄) and filtered. The filtrate was concentrated in vacuo and the residue was purified by chromatography on silica to give the title compound (1.19 g). ¹H NMR (300 MHz, CDCl₃) δ 6.98 (1H, s), 5.48 (2H, s), 3.82 (3H, s), 2.74-2.65 (1H, m), 1.25 (6H, d, J=6.7 Hz).

### Intermediate 15: 7-Isopropylthieno[3,2-d]pyrimidine-2,4(1H,3H)dione

A mixture of Intermediate 14 (1.19 g) and 2,2,2-trichloroacetyl isocyanate (1.56 g) in acetonitrile (60 mL) was stirred at room temperature for 30 minutes. The mixture was concentrated in vacuo and the residue was treated with a solution of ammonia in methanol (7 M, 20 mL) and the mixture was heated in the microwave at 70° C. for 15 minutes. After cooling, the mixture was concentrated in vacuo and the residue was dissolved in methanol (8 mL) and aqueous sodium hydroxide (1 M, 7.5 mL) was added. The mixture was heated in the microwave at 100° C. for 30 minutes. After cooling, the mixture was concentrated in vacuo and the residue was dissolved in water and acidified to pH 1 with concentrated hydrochloric acid. The solid was collected by filtration, washed with water and dried in vacuo over potassium hydroxide to give the title compound as a white solid (0.75 g). ¹H NMR (300 MHz, CDCl₃) δ 7.39 (1H, s), 3.02-2.94 (1H, m), 1.31 (6H, d, J=7.0 Hz).

### Intermediate 16: 2,4-Dichloro-7-isopropylthieno[3,2-d]pyrimidine

Intermediate 15 (50 mg) and POCl₃ (1 mL) were stirred at 100° C. for 3 hours. Then the reaction mixture was evaporated to near dryness and quenched with water. The product was washed with dichloromethane (×3). The combined organic layer was dried (MgSO₄) and concentrated in vacuo to give the title compound (65 mg). ¹H NMR (300 MHz, CDCl₃) δ 7.73 (d, J=0.9 Hz, 1H), 3.56-3.41 (m, 1H), 1.39 (d, J=7.4 Hz, 6H).

### Intermediate 17: 4-(2-((2-Chloro-7-isopropylthieno[3,2-d]pyrimidin-4-yl)amino)ethyl)phenol

To a mixture of Intermediate 16 (65 mg) and 4-(2-aminoethyl)phenol (34 mg) in ethanol (3 mL) DIPEA (0.69 mL) was added and the resulting mixture was stirred at room temperature for 6 hours. The reaction mixture was partitioned between water and ethyl acetate (×3). The combined organic layer was dried (MgSO₄) and concentrated in vacuo. The residue was purified by chromatography on silica eluting with 0-5% methanol in dichloromethane to give the title compound (56 mg). ¹H NMR (300 MHz, CDCl₃) δ 7.33 (d, J=1.2 Hz, 1H), 7.14-7.07 (m, 2H), 6.82-6.78 (m, 2H), 4.96 (br s, 1H), 4.83 (s, 1H), 3.87 (dt, J=5.9, 6.9 Hz, 2H), 3.48-3.39 (m, 1H), 2.92 (t, J=6.9 Hz, 2H), 1.33 (d, J=6.6 Hz, 6H);

### Compound Example 7: 4-(2-((2-(Benzo[b]thiophen-3-yl)-7-isopropylthieno[3,2-d]pyrimidin-4-yl)amino)ethyl)phenol

A mixture of Intermediate 17 (50 mg), benzo[b]thiophen-3-ylboronic acid (33 mg), potassium carbonate (50 mg), tetrakis(triphenylphosphine)palladium (0) (25 mg) in dioxane/water 20:1 mixture (5 mL) was stirred under microwave irradiation at 100° C. for 3 hours. The cooled reaction mixture was partitioned between water and ethyl acetate (×3). The combined organic layer was dried (MgSO₄) and concentrated in vacuo. The residue was purified by chromatography on silica eluting with 0-5% methanol in dichloromethane to give the title compound (36 mg). ¹H NMR (400 MHz, d6-DMSO) δ 9.28 (dd, J=1.4, 7.2 Hz, 1H), 9.18 (s, 1H), 8.66 (s, 1H), 8.06 (dd, J=1.4, 7.2 Hz, 1H), 7.93 (app t, J=5.6 Hz, 1H), 7.74 (d, J=1.4 Hz, 1H), 7.51-7.41 (m, 2H), 7.12 (d, J=8.4 Hz, 2H), 6.71 (d, J=8.4 Hz, 2H), 3.79 (app q, J=8.5 Hz, 2H), 3.48 (d, J=6.8 Hz, 1H), 2.91 (t, J=8.5 Hz, 2H), 1.41 (d, J=6.8 Hz, 6H); LCMS (Method 1) Rt 6.23 min m/z 446 [M+H]⁺.

### Compound Example 8: N-(2-(1H-Indol-3-yl)ethyl)-6-(benzo[b]thiophen-3-yl)-3-isopropylimidazo[1,5-a]pyrazin-8-amine

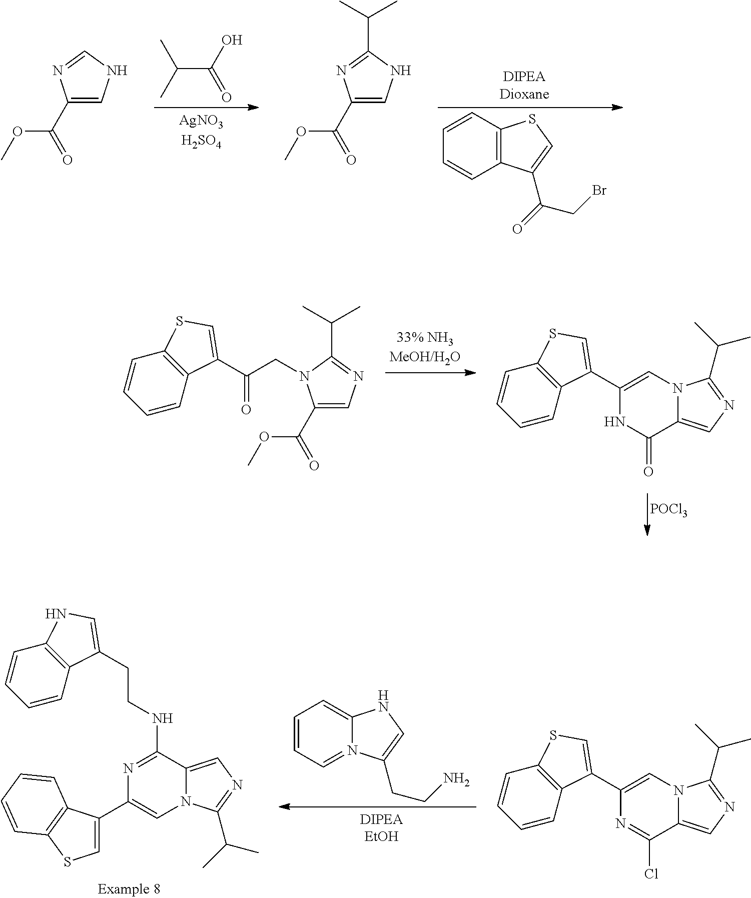

### Intermediate 18: Methyl 2-isopropyl-1H-imidazole-4-carboxylate

A solution of methyl 1H-imidazole-4-carboxylate (25 g), silver nitrate (20.2 g) and isobutyric acid (52.4 g) in H₂SO₄ (10% aqueous solution, 750 mL) was warmed to 80° C. for 15 minutes. An aqueous solution of ammonium persulphate (136 g, 595 mL) was added dropwise over 15 minutes at 80° C. The reaction mixture was allowed to cool to room temperature over 1 hour, cooled with ice and then basified to pH 11. The product was washed into ethyl acetate (×2). The combined organic layer was dried (MgSO₄) and concentrated in vacuo. The residue was purified by chromatography on silica eluting with 0-6% methanol in dichloromethane. The product was triturated with diethyl ether to give the title compound as a white solid (6.48 g). ¹H NMR (300 MHz, CDCl₃) δ 10.32 (br s, 1H), 7.64 (d, J=6.9 Hz, 1H), 3.87 (s, 3H), 3.13 (sept, J=6.6 Hz 1H), 1.38 (d, J=6.6 Hz, 6H).

### Intermediate 19: Methyl 1-(2-(benzo[b]thiophen-3-yl)-2-oxoethyl)-2-isopropyl-1H-imidazole-5-carboxylate

To a solution of intermediate 18 (25 g), silver nitrate (2.73 g) and 1-(benzo[b]thiophen-3-yl)-2-bromoethan-1-one (4.56 g) in dioxane (35 mL), DIPEA was added dropwise (5.7 mL) and the resulting mixture was stirred under microwave irradiation at 140° C. for 1 hour. The cooled reaction mixture was partitioned between water and ethyl acetate (×3). The combined organic layer was dried (MgSO₄) and concentrated in vacuo. The residue was purified by chromatography on silica eluting with 0-5% methanol/ammonia 2 N in dichloromethane to give the title compound (1.70 g). ¹H NMR (300 MHz, CDCl₃) δ 8.68-8.65 (m, 1H), 8.50 (s, 1H), 7.91-7.79 (m, 2H), 7.52-7.41 (m, 2H), 5.79 (br s, 2H), 3.76 (s, 3H), 2.89 (sept, J=6.8 Hz, 1H), 1.36 (d, J=6.8 Hz, 6H).

### Intermediate 20: 6-(Benzo[b]thiophen-3-yl)-3-isopropylimidazo[1,5-a]pyrazin-8(7H)-one

A solution of intermediate 19 (1.14 g) in 33% aqueous ammonia (18.14 g) was stirred under microwave irradiation at 100° C. for 1 hour. The cooled reaction mixture was concentrated in vacuo and the residue was triturated in ethyl acetate to give the title compound (0.6 g). ¹H NMR (300 MHz, CDCl₃) δ 8.61 (br s, 1H), 7.98-7.93 (m, 3H), 7.76 (s, 1H), 7.51-7.45 (m, 2H), 7.15 (s, 1H), 3.24 (sept, J=6.8 Hz, 1H), 1.46 (d, J=6.8 Hz, 6H).

### Intermediate 21: 6-(Benzo[b]thiophen-3-yl)-8-chloro-3-isopropylimidazo[1,5-a]pyrazine

Intermediate 20 (0.59 g) and POCl₃ (9 mL) were stirred at 100° C. for 3 hours. The cooled reaction mixture was evaporated to near dryness and partitioned between sodium bicarbonate saturated aqueous solution and ethyl acetate (×3). The combined organic layer was dried (MgSO₄) and concentrated in vacuo to give a brown solid (0.84 g). The residue was purified by chromatography on silica eluting with 0-50% ethyl acetate in cyclohexane to give the title compound (0.17 g). ¹H NMR (300 MHz, CDCl₃) δ 8.19-8.14 (m, 1H), 7.96-7.91 (m, 2H), 7.85-7.83 (m, 2H), 7.52-7.41 (m, 2H), 3.39 (sept, J=7.3 Hz, 1H), 1.51 (d, J=7.3 Hz, 6H).

### Compound Example 8: N-(2-(1H-Indol-3-yl)ethyl)-6-(benzo[b]thiophen-3-yl)-3-isopropylimidazo[1,5-a]pyrazin-8-amine

A mixture of Intermediate 21 (90 mg), tryptamine (88 mg) and DIPEA (0.14 mL) in ethanol (3 mL) was stirred under microwave irradiation at 120° C. for 2 hours. The cooled reaction mixture was concentrated in vacuo. The residue was purified by chromatography on silica eluting with 0-5% methanol in dichloromethane to give the title compound (85 mg). ¹H NMR (400 MHz, d6-DMSO) δ 10.81 (br s, 1H), 8.48 (d, J=7.8 Hz, 1H), 8.05-8.03 (m, 2H), 7.90-7.85 (m, 2H), 7.73 (s, 1H), 7.57 (d, J=7.8 Hz, 1H), 7.43-7.30 (m, 3H), 7.18 (d, J=2.0 Hz, 1H), 7.06-7.01 (m, 1H), 6.85-6.80 (m, 1H), 3.85-3.78 (m, 2H), 3.52 (sept, J=6.8 Hz, 1H), 3.09 (t, J=7.6 Hz, 2H), 1.33 (d, J=6.8 Hz, 6H); LCMS (Method 1) Rt 4.45 min m/z 452 [M+H]⁺.

### Compound Example 9: 4-(2-((6-(Benzo[b]thiophen-3-yl)-3-isopropylimidazo[1,5-a]pyrazin-8-yl)amino)ethyl)phenol

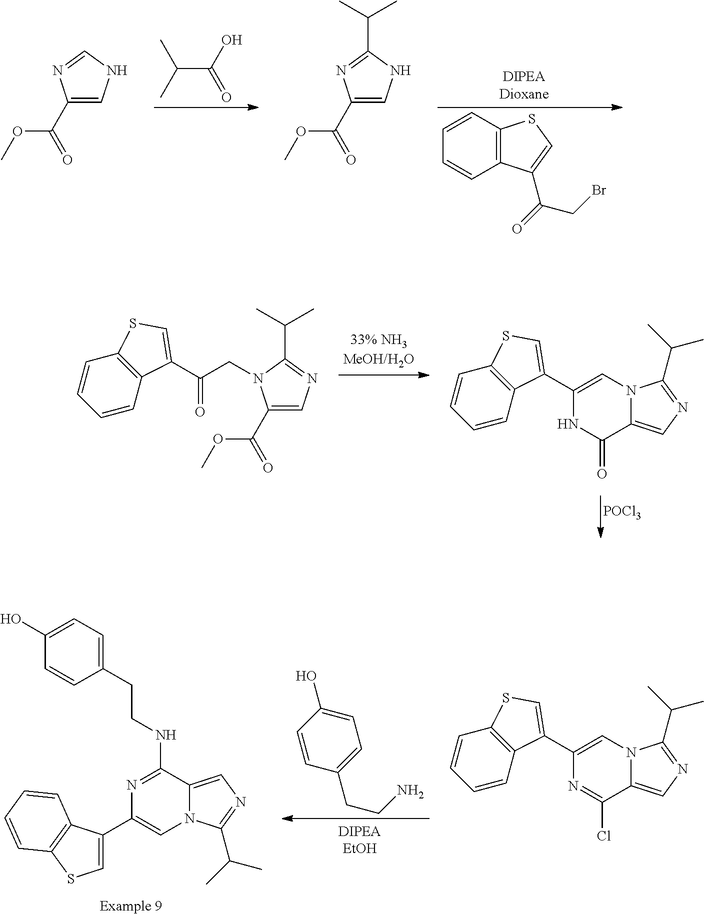

### Compound Example 9: 4-(2-((6-(Benzo[b]thiophen-3-yl)-3-isopropylimidazo[1,5-a]pyrazin-8-yl)amino)ethyl)phenol

A mixture of Intermediate 21 (80 mg), tyramine (67 mg) and DIPEA (0.13 mL) in ethanol (3 mL) was stirred under microwave irradiation at 100° C. for 8 hours. The cooled reaction mixture was concentrated in vacuo and partitioned between water and ethyl acetate (×3). The combined organic layer was dried (MgSO₄) and concentrated in vacuo. The residue was purified by chromatography on silica eluting with 0-10% methanol in dichloromethane to give the title compound (75mg). ¹H NMR (400 MHz, DMSO) δ 9.16 (s, 1H), 8.53-8.48 (m, 1H), 8.06-8.05 (m, 2H), 7.86 (s, 1H), 7.81 (app t, J=5.6 Hz, 1H), 7.71 (s, 1H), 7.43-7.40 (m, 2H), 7.10 (d, J=8.2 Hz, 2H), 6.67 (d, J=8.2 Hz, 2H), 3.71-3.64 (m, 2H), 3.51 (sept, J=6.2 Hz, 1H), 2.91-2.84 (m, 2H), 1.33 (d, J=6.2 Hz, 6H); LCMS (Method 1) Rt 4.01 min m/z 429 [M+H]⁺.

### Compound Example 10: 5-(4-((2-(1H-Indol-3-yl)ethyl)amino)-7-isopropylthieno[3,2-d]pyrimidin-2-yl)nicotinonitrile

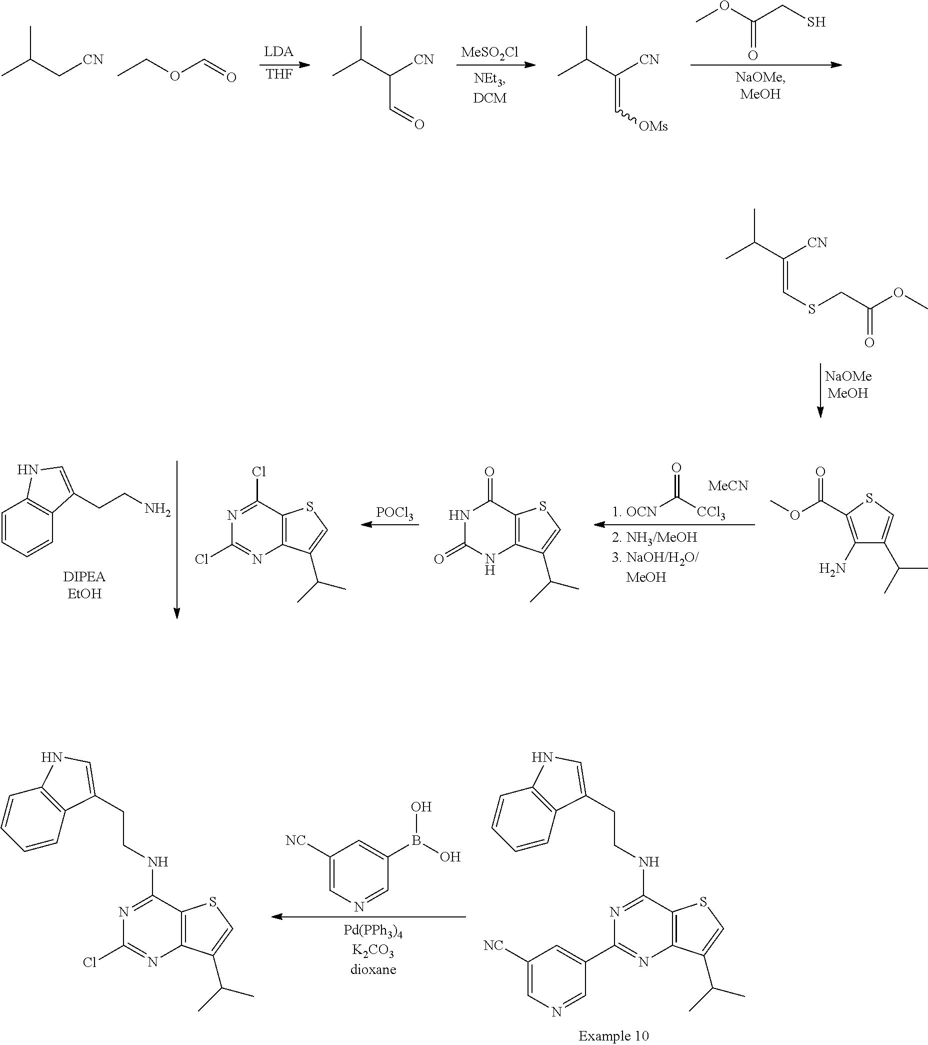

### Intermediate 22: N-(2-(1H-Indol-3-yl)ethyl)-2-chloro-7-isopropylthieno[3,2-d]pyrimidin-4-amine

To a mixture of Intermediate 16 (360 mg) and tryptamine (257 mg) in ethanol (8 mL) DIPEA (0.76 mL) was added and the resulting mixture was stirred at room temperature for 1.5 hours. The reaction mixture was partitioned between water and ethyl acetate (×3). The combined organic layer was dried (MgSO₄) and concentrated in vacuo. The residue was purified by chromatography on silica eluting with 0-4% methanol in dichloromethane with 1% triethylamine to give the title compound (0.45 g). ¹H NMR (300 MHz, CDCl₃) δ 8.42 (s, 1H), 7.70-7.66 (m, 1H), 7.40-7.37 (m, 1H), 7.30-7.12 (m, 3H), 7.06 (d, J=2.7 Hz, 1H), 5.13 (br s, 1H), 3.97 (app q, J=6.4 Hz, 2H), 3.50-3.35 (m, 1H), 3.15 (t, J=6.4 Hz, 2H), 1.32 (d, J=6.8 Hz, 6H).

### Compound Example 10: 5-(4-((2-(1H-Indol-3-yl)ethyl)amino)-7-isopropylthieno[3,2-d]pyrimidin-2-yl)nicotinonitrile

A mixture of Intermediate 22 (100 mg), (5-cyanopyridin-3-yl)boronic acid (96 mg), potassium carbonate (93 mg), tetrakis(triphenylphosphine)palladium (0) (31 mg) in dioxane/water 5:1 mixture (6 mL) was stirred under microwave irradiation at 90° C. for 30 minutes. The cooled reaction mixture was partitioned between water and ethyl acetate (×3). The combined organic layer was dried (MgSO₄) and concentrated in vacuo. The residue was purified by chromatography on silica eluting with 0-5% methanol in dichloromethane to give the title compound (85 mg). ¹H NMR (400 MHz, d6-DMSO) δ 10.83 (s, 1H), 9.78 (d, J=2.1 Hz, 1H), 9.12 (d, J=2.1 Hz, 1H), 9.00 (app t, J=2.1 Hz, 1H), 8.20 (app t, J=5.6 Hz, 1H), 7.78 (d, J=1.1 Hz, 1H), 7.65 (d, J=7.6 Hz, 1H), 7.33 (d, J=7.6 Hz, 1H), 7.21 (d, J=2.3 Hz, 1H), 7.09-6.98 (m, 2H), 3.96-3.88 (m, 2H), 3.50-3.42 (m, 1H), 3.12 (t, J=7.6 Hz, 2H), 1.38 (d, J=7.6 Hz, 6H); LCMS (Method 1) Rt 6.04 min m/z 439 [M+H]⁺.

### Compound Example 11: N-(2-(1H-Indol-3-yl)ethyl)-2-(5-fluoropyridin-3-yl)-'7-isopropylthieno[3,2-d]pyrimidin-4-amine

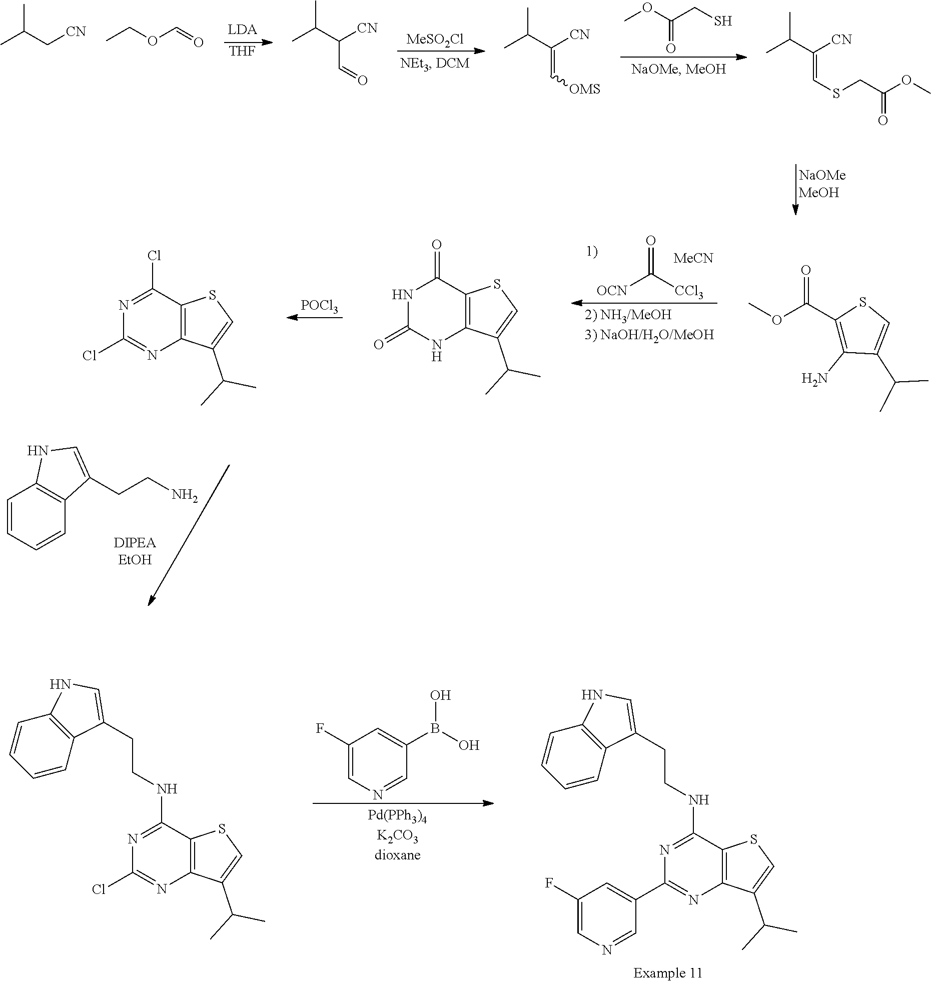

### Compound Example 11: N-(2-(1H-Indol-3-yl)ethyl)-2-(5-fluoropyridin-3-yl)-'7-isopropylthieno[3,2-d]pyrimidin-4-amine

A mixture of Intermediate 22 (90 mg), (5-fluoropyridin-3-yl)boronic acid (68 mg), potassium carbonate (84 mg), tetrakis(triphenylphosphine)palladium (0) (28 mg) in dioxane/water 5:1 mixture (6 mL) was stirred under microwave irradiation at 100° C. for 2 hours. The cooled reaction mixture was partitioned between water and ethyl acetate (×3). The combined organic layer was dried (MgSO₄) and concentrated in vacuo. The residue was purified by chromatography on silica eluting with 0-5% methanol in dichloromethane to give the title compound (95 mg) ¹H NMR (400 MHz, d6-DMSO) 10.84 (s, 1H), 9.46 (app t, J=1.6 Hz, 1H), 8.69 (d, J=3.0 Hz, 1H), 8.46-8.42 (m, 1H), 8.17 (app t, J=5.6 Hz, 1H), 7.77 (d, J=0.8 Hz, 1H), 7.64 (d, J=8.1 Hz, 1H), 7.34 (d, J=8.1 Hz, 1H), 7.22 (d, J=2.3 Hz, 1H), 7.10-6.96 (m, 2H), 3.92 (app q, J=6.4 Hz, 2H), 3.50-3.39 (m, 1H), 3.12 (t, J=7.6 Hz, 2H), 1.38 (d, J=6.4 Hz, 6H); LCMS (Method 1) Rt 6.23 min m/z 432 [M+H]⁺.

### Compound Example 12: N-(2-(1H-Indol-3-yl)ethyl)-2-(5-fluoropyridin-3-yl)furo[3,2-d]pyrimidin-4-amine

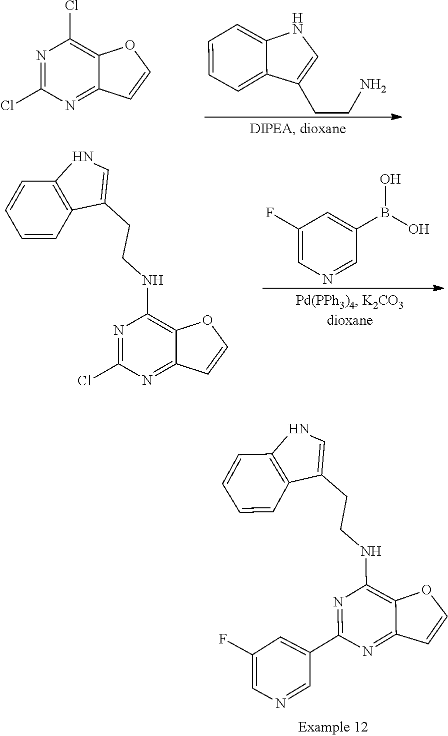 [SMILES: Clc1nc(Cl)c2occc2n1 ; NCCc1c[nH]c2ccccc12 ; Clc1nc(NCCc2c[nH]c3ccccc23)c2occc2n1 ; OB(O)c1cncc(F)c1 ; Fc1cncc(-c2nc(NCCc3c[nH]c4ccccc34)c3occc3n2)c1]

### Intermediate 23: N-(2-(1H-indol-3-yl)ethyl)-2-chlorofuro[3,2-d]pyrimidin-4-amine

To a mixture of 2,4-dichlorofuro[3,2-d]pyrimidine (0.90 g) and tryptamine (0.763 g) in dioxane (16 mL) DIPEA (1.7 mL) was added dropwise and the resulting mixture was stirred under microwave irradiation at 60° C. for 1 hour. The cooled reaction mixture was partitioned between water and ethyl acetate (×3). The combined organic layer was dried (MgSO₄) and concentrated in vacuo. The residue was purified by chromatography on silica eluting with 0-10% methanol in dichloromethane to give the title compound (0.30 g). ¹H NMR (300 MHz, d6-DMSO) δ 10.83 (br s, 1H), 8.53 (br s, 1H), 8.27 (d, J=1.8 Hz, 1H), 7.66 (br d, J=8.0 Hz, 1H), 7.36-7.32 (m, 1H), 7.20 (d, J=2.2 Hz, 1H), 7.10-7.01 (m, 2H), 6.96 (d, J=2.2 Hz, 1H), 3.74-3.64 (br m, 2H), 3.03 (t, J=7.4 Hz, 2H); LCMS (Method 3) Rt 1.38 min m/z 312.9-314.9 [M+H]⁺.

### Compound Example 12: N-(2-(1H-Indol-3-yl)ethyl)-2-(5-fluoropyridin-3-yl)furo[3,2-d]pyrimidin-4-amine

A mixture of Intermediate 23 (100 mg), (5-fluoropyridin-3-yl)boronic acid (90 mg), potassium carbonate (110 mg), tetrakis(triphenylphosphine)palladium (0) (37 mg) in dioxane/water 2:1 mixture (3 mL) was stirred under microwave irradiation at 90° C. for 45 minutes. The cooled reaction mixture was partitioned between water and ethyl acetate (×3). The combined organic layer was dried (MgSO₄) and concentrated in vacuo. The residue was purified by chromatography on silica eluting with 0-8% methanol in dichloromethane to give the semicrude product (110 mg). The product was purified in MDAP under acidic conditions to afford the title compound as a white solid (28 mg). ¹H NMR (400 MHz, DMSO) δ 10.83 (s, 1H), 9.38 (s, 1H), 8.66 (d, J=2.7 Hz, 1H), 8.40-8.35 (m, 1H), 8.29 (d, J=2.7 Hz, 1H), 8.26 (br s, 1H), 7.63 (d, J=8.1 Hz, 1H), 7.34 (d, J=8.1 Hz, 1H), 7.23 (d, J=2.3 Hz, 1H), 7.08-6.97 (m, 3H), 3.89 (app q, J=7.5 Hz, 2H), 3.10 (t, J=7.5 Hz, 2H); LCMS (Method 1) Rt 4.47 min m/z 374 [M+H]⁺.

### Compound Example 13: N-(2-(1H-Indol-3-yl)ethyl)-2-(5-methylpyridin-3-yl)furo[3,2-d]pyrimidin-4-amine

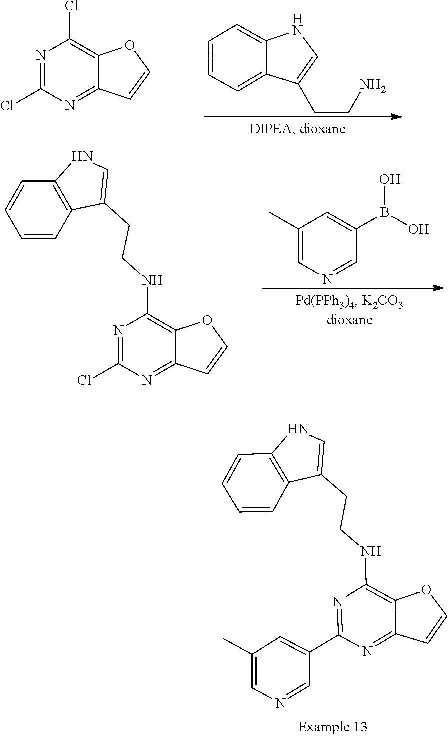

### Compound Example 13: N-(2-(1H-Indol-3-yl)ethyl)-2-(5-methylpyridin-3-yl)furo[3,2-d]pyrimidin-4-amine

A mixture of Intermediate 23 (100 mg), (5-methylpyridin-3-yl)boronic acid (88 mg), potassium carbonate (110 mg), tetrakis(triphenylphosphine)palladium (0) (37 mg) in dioxane/water 2:1 mixture (3 mL) was stirred under microwave irradiation at 90° C. for 45 minutes. The cooled reaction mixture was partitioned between water and ethyl acetate (×3). The combined organic layer was dried (MgSO₄) and concentrated in vacuo. The residue was purified by chromatography on silica eluting with 0-8% methanol in dichloromethane to give the semicrude product (50 mg). The product was purified in MDAP under acidic conditions to afford the title compound as a white solid (27 mg). ¹H NMR (400 MHz, d6-DMSO) δ 10.84 (s, 1H), 9.32 (d, J=2.1 Hz, 1H), 8.48-8.45 (m, 2H), 8.26 (d, J=2.1 Hz, 1H), 8.16 (br s, 1H), 7.64 (d, J=8.4 Hz, 1H), 7.34 (d, J=8.4 Hz, 1H), 7.23 (d, J=2.3 Hz, 1H), 7.10-6.95 (m, 3H), 3.91 (app q, J=7.8 Hz, 2H), 3.11 (t, J=7.8 Hz, 2H), 2.40 (s, 3H); LCMS (Method 1) Rt 3.32 min m/z 370 [M+H]⁺.

### Compound Example 14: 5-(4-((2-(1H-Indol-3-yl)ethyl)amino)furo[3,2-d]pyrimidin-2-yl)nicotinonitrile

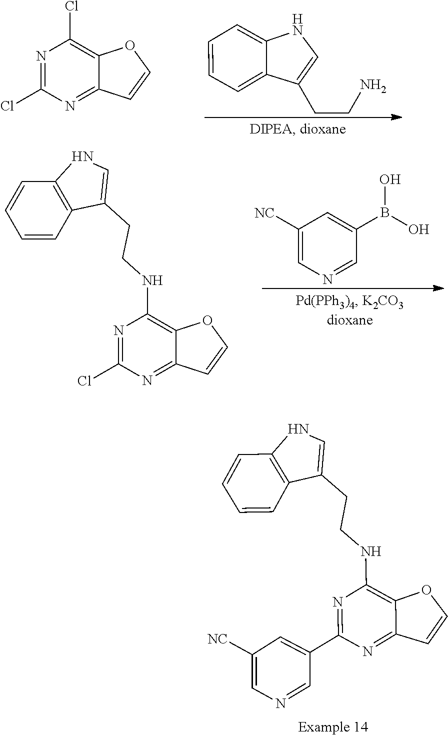 [SMILES: Clc1nc(Cl)c2occc2n1 ; NCCc1c[nH]c2ccccc12 ; Clc1nc(NCCc2c[nH]c3ccccc23)c2occc2n1 ; N#Cc1cncc(B(O)O)c1 ; N#Cc1cncc(-c2nc(NCCc3c[nH]c4ccccc34)c3occc3n2)c1]

### Compound Example 14: 5-(4-((2-(1H-Indol-3-yl)ethyl)amino)furo[3,2-d]pyrimidin-2-yl)nicotinonitrile

A mixture of Intermediate 23 (100 mg), (5-cyanopyridin-3-yl)boronic acid (113 mg), potassium carbonate (110 mg), tetrakis(triphenylphosphine)palladium (0) (37 mg) in dioxane/water 2:1 mixture (3 mL) was stirred under microwave irradiation at 90° C. for 45 minutes. The cooled reaction mixture was partitioned between water and ethyl acetate (×3). The combined organic layer was dried (MgSO₄) and concentrated in vacuo. The residue was purified by chromatography on silica eluting with 0-8% methanol in dichloromethane to the semicrude compound (125 mg). The product was purified in MDAP under acidic conditions to afford the title compound as a white solid (50 mg). ¹H NMR (400 MHz, d6-DMSO) δ 10.82 (s, 1H), 9.70 (d, J=2.0 Hz, 1H), 9.10 (d, J=2.0 Hz, 1H), 8.93 (app t, J=2.0 Hz, 1H), 8.30-8.26 (m, 2H), 7.63 (d, J=7.5 Hz, 1H), 7.32 (d, J=7.5 Hz, 1H), 7.22 (d, J=2.3 Hz, 1H), 7.08 (d, J=2.3 Hz, 1H), 7.07-6.98 (m, 2H), 3.95-3.86 (m, 2H), 3.10 (t, J=7.5 Hz, 2H); LCMS (Method 1) Rt 4.50 min m/z 381 [M+H]⁺.

### Compound Example 15: N-(2-(1H-Indol-3-yl)ethyl)-7-isopropyl-2-(5-methylpyridin-3-yl)thieno[3,2-d]pyrimidin-4-amine

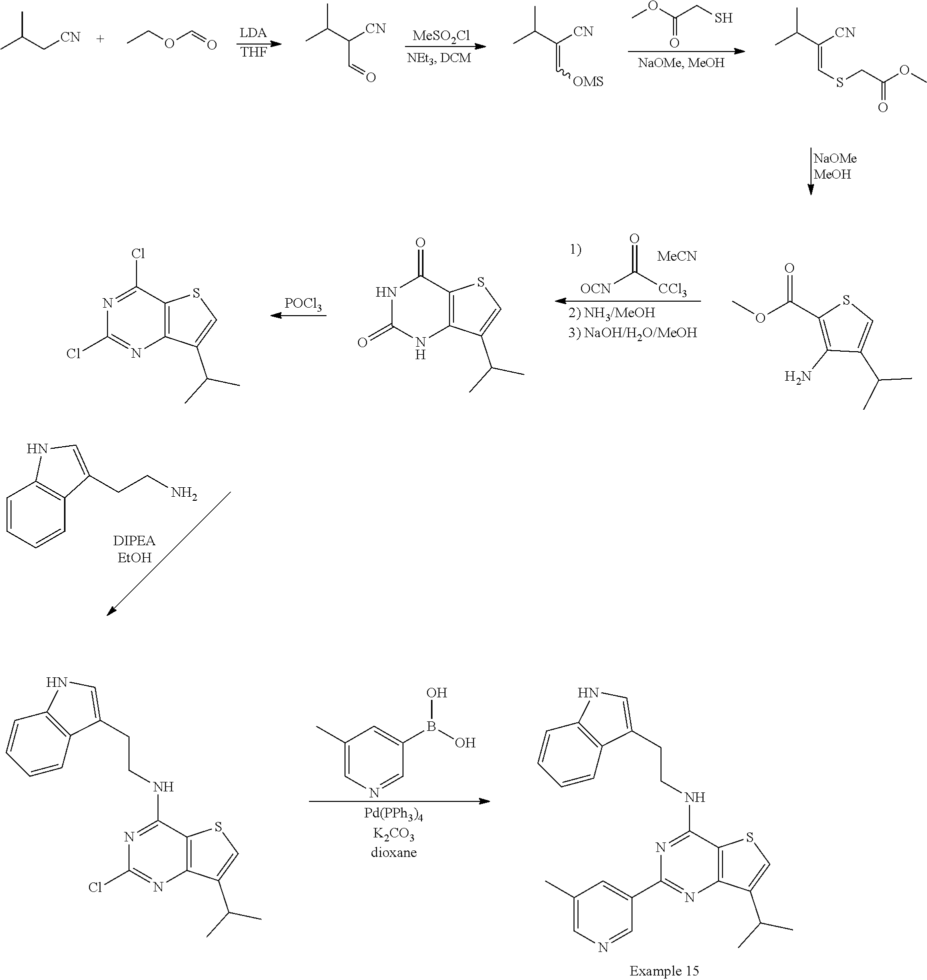

### Compound Example 15: N-(2-(1H-Indol-3-yl)ethyl)-7-isopropyl-2-(5-methylpyridin-3-yl)thieno[3,2-d]pyrimidin-4-amine

A mixture of Intermediate 22 (90 mg), (5-methylpyridin-3-yl)boronic acid (66 mg), potassium carbonate (84 mg), tetrakis(triphenylphosphine)palladium (0) (28 mg) in dioxane/water 5:1 mixture (6 mL) was stirred under microwave irradiation at 100° C. for 2 hours. The cooled reaction mixture was acidified at pH 3 with 1 N HCl solution and re-basified at pH 13 by addition of solid potassium carbonate. The product was washed into ethyl acetate (×3). The combined organic layer was dried (MgSO₄) and concentrated in vacuo. The residue was purified by chromatography on silica eluting with 0-5% methanol in dichloromethane to give the semicrude product (100 mg). The product was purified in MDAP under acidic conditions to afford the title compound as a white solid (36 mg). ¹H NMR (400 MHz, d6-DMSO) δ 10.85 (s, 1H), 9.41 (d, J=1.0 Hz, 1H), 8.52-8.51 (m, 2H), 8.08 (app t, J=5.6 Hz, 1H), 7.74 (d, J=1.0 Hz, 1H), 7.65 (d, J=8.2 Hz, 1H), 7.35 (d, J=8.2 Hz, 1H), 7.23 (d, J=2.2 Hz, 1H), 7.11-6.96 (m, 2H), 3.90 (app q, J=8.4 Hz, 2H), 3.50-3.40 (m, 1H), 3.13 (t, J=8.4 Hz, 2H), 2.41 (s, 3H), 1.39 (d, J=7.4 Hz, 6H); LCMS (Method 1) Rt 4.56 min m/z 428 [M+H]⁺.

### Compound Example 16: 4-(2-((2-(Benzo[b]thiophen-3-yl)-6-(isopropylamino)pyrimidin-4-yl)amino)ethyl)phenol

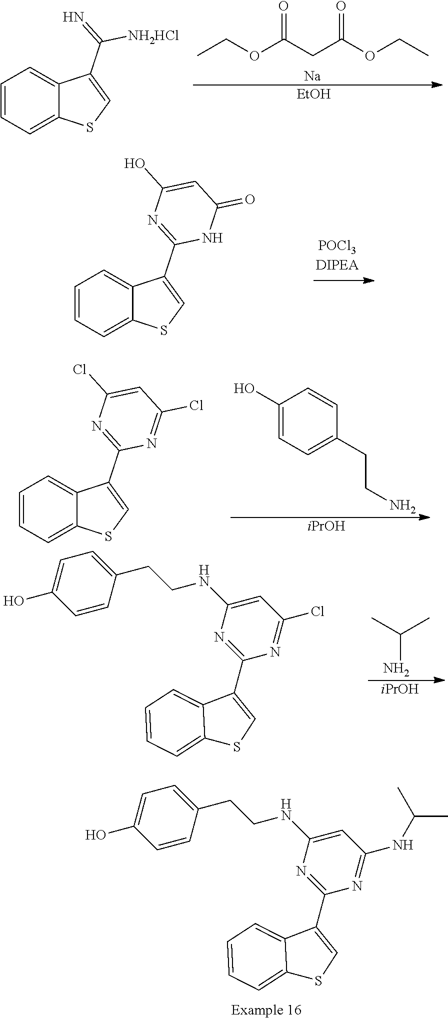

### Intermediate 24: 2-(Benzo[b]thiophen-3-yl)-6-hydroxypyrimidin-4(3H)-one

Sodium (0.23 g) was added to pure ethyl alcohol (20 mL) under argon atmosphere and stirred until completely dissolved. Benzo[b]thiophene-3-carboximidamide hydrochloride (1.00 g) and diethyl malonate (0.714 mL) were then added and the resulting mixture was allowed to stir at reflux for 3 hours. The cooled reaction mixture was cooled to room temperature and the solvent was removed in vacuo. The residue was diluted with water and acidified with aqueous HCl 1 M. The resulting white solid was isolated by filtration and washed with water, IMS and diethyl ether. Then the residue was dried at 60-65° C. under vacuum overnight to afford the title compound as a white solid (0.778 g). ¹H NMR (400 MHz, d4-MeOH) δ 8.71-8.68 (m, 1H), 8.40 (s, 1H), 8.07-7.99 (m, 2H), 7.59-7.40 (m, 3H); LCMS (Method 3) Rt 1.08 min m/z 245 [M+H]⁺.

### Intermediate 25: 2-(Benzo[b]thiophen-3-yl)-4,6-dichloropyrimidine

Intermediate 24 (0.50 g) was suspended in phosphorus(V) oxychloride (3.2 mL) then DIPEA (0.35 mL) was added dropwise and the resulting mixture was heated at reflux for 4 hours. The cooled reaction mixture was cooled to room temperature and the solvent was removed in vacuo. The residue was basified with NaHCO₃ saturated aqueous solution and extracted with ethyl acetate. The combined extracts were dried (MgSO₄) and concentrated in vacuo to afford a beige solid. The residue was triturated with diethyl ether to afford the title compound as a beige solid (0.287 g). ¹H NMR (400 MHz, CDCl₃) δ 9.05 (d, J=8.1 Hz, 1H), 8.76 (s, 1H), 7.91 (d, J=8.1 Hz, 1H), 7.57-7.42 (m, 3H); LCMS (Method 3) Rt 1.81 min m/z 283/285 [M+H]⁺.

### Intermediate 26: 4-(2-((2-(Benzo[b]thiophen-3-yl)-6-chloropyrimidin-4-yl)amino)ethyl)phenol

Intermediate 25 (283 mg) in 2-propanol (6.0 mL) was treated with tyramine (152 mg) and the resulting mixture was stirred at 50° C. for 2 hours and then at reflux for 2 hours. The solvent was removed in vacuo to afford a beige solid which was triturated with diethyl ether. The solid was discarded and the diethyl ether was concentrated in vacuo to give a light brown solid as semicrude product. The residue was purified by chromatography on silica eluting with 0-50% ethyl acetate in iso-hexane to afford the title compound as a light yellow oil (176 mg). ¹H NMR (400 MHz, CDCl₃) δ 9.03 (br d, J=7.9 Hz, 1H), 8.53 (s, 1H), 7.89 (d, J=7.9 Hz, 1H), 7.49-7.36 (m, 2H), 7.11 (d, J=8.4 Hz, 2H), 6.81 (d, J=8.4 Hz, 2H), 6.23 (s, 1H), 4.98 (s, 2H), 2.92 (t, J=7.1 Hz, 2H), 1.26 (t, J=7.1 Hz, 2H); LCMS (Method 3) Rt 1.68 min m/z 382 [M+H]⁺.

### Compound Example 16: 4-(2-((2-(Benzo[b]thiophen-3-yl)-6-(isopropylamino)pyrimidin-4-yl)amino)ethyl)phenol

A mixture of Intermediate 26 (172 mg) and isopropylamine (160 mg) in 2-propanol (4.0 mL) was sealed in a vial and heated under microwave irradiation at 100° C. overnight. Further 1 mL of isopropylamine was added and the vial was re-irradiated at 130° C. for 1 hour, followed by 150° C. for 5 hours. The mixture was concentrated in vacuo, re-dissolved in neat isopropylamine and re-irradiated at 100° C. for 1 hour, followed by irradiation at 140° C. for 30 minutes. The solvent was removed in vacuo to afford a light brown oil. The residue was purified by chromatography on silica eluting with 0-25% ethyl acetate in iso-hexane to give the title compound (88 mg). ¹H NMR (400 MHz, d6-DMSO) δ 9.19-9.14 (m, 2H), 8.44 (s, 1H), 8.02-7.98 (m, 1H), 7.44-7.35 (m, 2H), 7.08 (d, J=8.5 Hz, 2H), 6.72-6.65 (m, 3H), 6.47 (br d, J=7.5 Hz, 1H), 5.36 (s, 1H), 4.06-3.98 (br m, 1H), 3.42 (br s, 2H), 2.77 (t, J=7.8 Hz, 2H), 1.19 (d, J=6.6 Hz, 6H); LCMS (Method 1) Rt 3.55 min m/z 405 [M+H]⁺.

### Compound Example 17: 5-(2-42-(1H-Indol-3-yl)ethyl)amino)-6-(sec-butylamino)pyrimidin-4-yl)nicotinonitrile

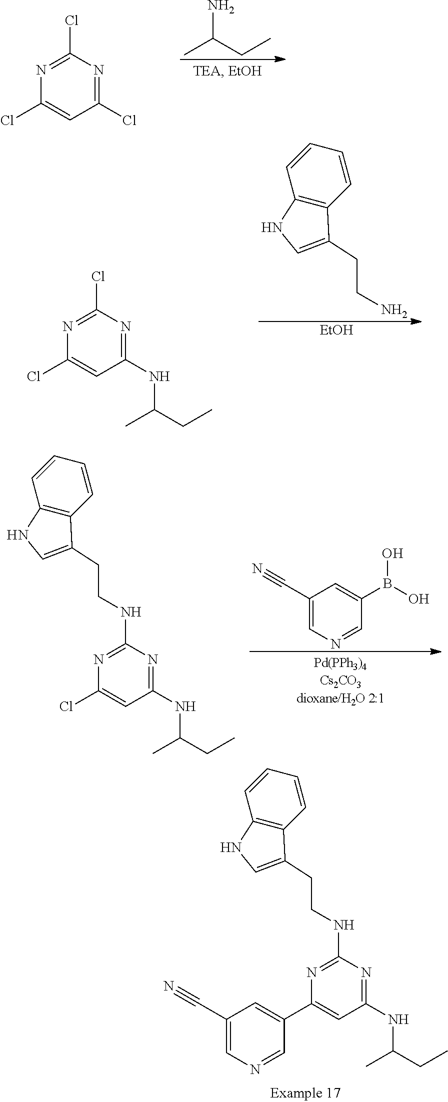

### Intermediate 27: N-(sec-Butyl)-2,6-dichloropyrimidin-4-amine

To a solution of 2,4,6-trichloropyrimidine (1.00 g), TEA (0.795 mL) and pure ethyl alcohol (10.0 mL), sec-butylamine (0.551 mL) was added dropwise and the resulting mixture was allowed to stir at room temperature overnight. The solvent was removed in vacuo and the residue was diluted with ethyl acetate, washed with water, brine, dried (MgSO₄) and concentrated in vacuo to afford a colourless oil. The residue was purified by chromatography on silica eluting with 0-50% ethyl acetate in iso-hexane to afford the title compound as a colourless oil (0.693 g). ¹H NMR (400 MHz, CDCl₃) δ 6.25 (s, 1H), 5.30 (s, 1H), 1.61-1.53 (m, 2H), 1.28-1.17 (m, 4H), 0.96 (t, J=7.8 Hz, 3H); LCMS (Method 3) Rt 1.42 min m/z 221.8-223.8 [M+H]⁺.

### Intermediate 28: N²-(2-(1H-Indol-3-yl)ethyl)-N⁴-(sec-butyl)-6-chloropyrimidine-2,4-diamine

Intermediate 27 (0.200 g), tryptamine (0.176 g) and pure ethyl alcohol (10.0 mL) were stirred at room temperature for 2 hours. The solvent was removed in vacuo and the residue was purified by chromatography on silica eluting with 0-50% ethyl acetate in iso-hexane to afford the title compound as a colourless oil (0.054 g). ¹H NMR (400 MHz, CDCl₃) δ 8.05 (br s, 1H), 7.65 (d, J=8.1 Hz, 1H), 7.37 (dt, J=1.1, 5.0 Hz, 1H), 7.23-7.10 (m, 2H), 7.04 (d, J=2.5 Hz, 1H), 5.67 (s, 1H), 4.99 (br s, 1H), 4.56 (br s, 1H), 3.73-3.66 (m, 2H), 3.03 (t, J=6.8 Hz, 2H), 1.58-1.49 (m, 3H), 1.18 (d, J=4.8 Hz, 3H), 0.94 (t, J=6.9 Hz, 3H); LCMS (Method 3) Rt 1.45 min m/z 344 [M+H]⁺.

### Compound Example 17: 5-(24(2-(1H-Indol-3-yl)ethyl)amino)-6-(sec-butylamino)pyrimidin-4-yl)nicotinonitrile

Intermediate 28 (54 mg), 5-(2-((2-(1H-indol-3-yl)ethyl)amino)-6-(sec-butylamino)pyrimidin-4-yl)nicotinonitrile (27.9 mg), tetrakis(triphenylphosphine)palladium (0) (18.2 mg) and cesium carbonate (153 mg) were suspended in dioxane/water 2:1 (3 mL) and the resulting mixture was stirred under microwave irradiation at 120° C. for 1 hour. The cooled reaction mixture was concentrated in vacuo, re-diluted in ethyl acetate and washed with water and brine. The combined organic layer was dried (MgSO₄) and concentrated in vacuo. The residue was purified by chromatography on silica eluting with 0-100% ethyl acetate in iso-hexane to give a yellow gum. The residue was purified by HPLC (Kinetix Acid C18 RP short, 30-98% CH₃CN/H₂O [0.1% HCO₂H] @18 mL/min over 5 min gradient) and freeze-dried to afford the title compound as a white solid (24 mg). ¹H NMR (400 MHz, d6-DMSO) δ 10.79 (s, 1H), 9.33 (br s, 1H), 9.07 (d, J=2.0 Hz, 1H), 8.70 (br s, 1H), 7.59 (d, J=8.2 Hz, 1H), 7.34 (d, J=8.2 Hz, 1H), 7.18 (d, J=2.0 Hz, 1H), 7.09-6.94 (m, 2H), 6.74 (br s, 2H), 6.34 (br s, 1H), 4.11-4.05 (br m, 1H), 3.63-3.53 (br m, 2H), 2.96 (t, J=7.6 Hz, 2H), 1.56-1.47 (m, 2H), 1.14 (br d, J=5.5 Hz, 3H), 0.89 (t, J=6.0 Hz, 3H); LCMS (Method 1) Rt 3.68 min m/z 412 [M+H]⁺.

### Compound Example 18: 4-(2-((2-(Benzo[b]thiophen-3-yl)-7-isopropyl-6,7-dihydro-5H-pyrrolo[2,3-d]pyrimidin-4-yl)amino)ethyl)phenol

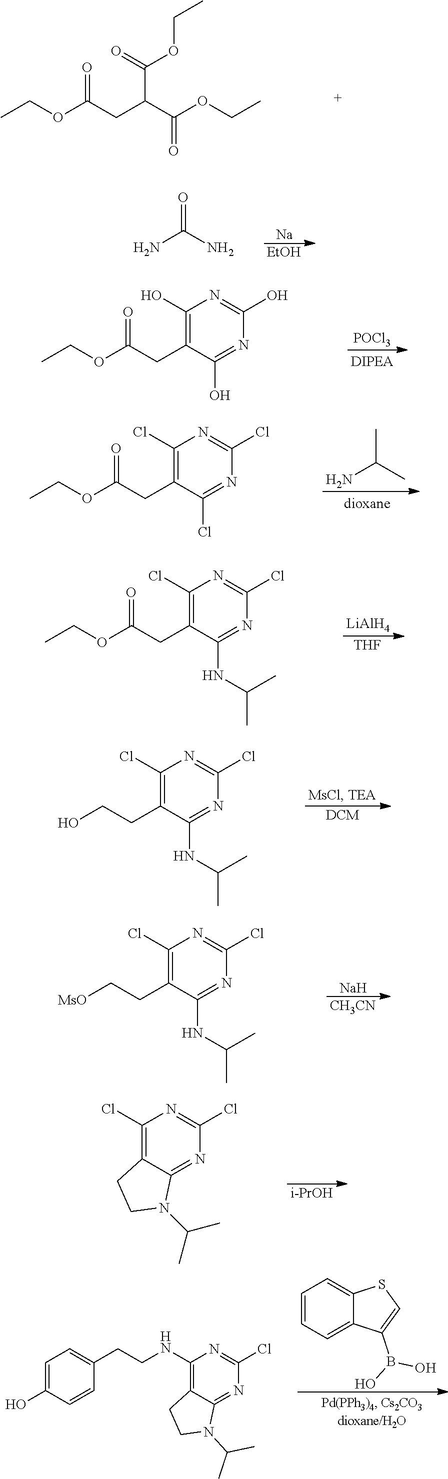

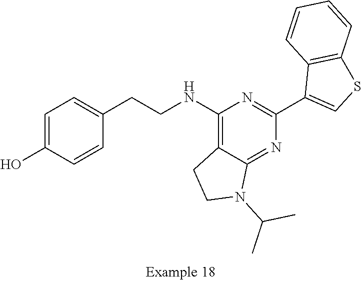 [SMILES: CC(C)N1CCc2c(NCCc3ccc(O)cc3)nc(-c3csc4ccccc34)nc21]

### Intermediate 29: Ethyl 2-(2,4,6-trihydroxypyrimidin-5-yl)acetate

Sodium (1.36 g) was dissolved in pure ethyl alcohol (150 mL) at room temperature. Then urea (2.38 g) and triethyl ethane-1,1,2-tricarboxylate (9.1 mL) were then added and the resulting mixture was allowed to stir at reflux for overnight. The cooled reaction mixture was cooled to room temperature and the solvent was removed in vacuo. The residue was diluted with water and acidified with aqueous HCl 1 M. The aqueous phase was washed with dichloromethane and ethyl acetate, then concentrated in vacuo to afford the title compound as a solid (9.04 g, quantitative). ¹H NMR (400 MHz, DMSO) δ 9.21-9.01 (br m, 3H), 3.95 (q, J=7.6 Hz, 2H), 3.01 (s, 2H), 1.14 (t, J=7.6 Hz, 3H); LCMS (Method 3) Rt 0.70 min m/z 215 [M+H]⁺.

### Intermediate 30: Ethyl 2-(2,4,6-trichloropyrimidin-5-yl)acetate

Intermediate 29 (4.00 g) was suspended in phosphorus(V) oxychloride (30 mL) then DIPEA (3.2 mL) was added dropwise and the resulting mixture was heated at reflux for 18 hours. The cooled reaction mixture was concentrated in vacuo. The residue was cooled to 0-5° C. and basified with NaHCO₃ saturated aqueous solution. The aqueous phase was extracted with ethyl acetate. The combined extracts were washed with brine and then dried (MgSO₄) and concentrated in vacuo to afford a brown oil. The residue was purified by chromatography on silica eluting with 0-50% ethyl acetate in iso-hexane to give the title compound as a pale yellow oil (1.33 g). ¹H NMR (400 MHz, CDCl₃) δ 4.23 (q, J=7.1 Hz, 2H), 3.95 (s, 2H), 1.29 (t, J=7.1 Hz, 3H).

### Intermediate 31: Ethyl 2-(2,4-dichloro-6-(isopropylamino)pyrimidin-5-yl)acetate

Intermediate 30 (0.5 g) was dissolved in dioxane (4.0 mL) and then isopropylamine (0.4 mL) was added dropwise and the resulting mixture was stirred at room temperature for 3 hours. The solvent was removed in vacuo and the residue was diluted with dichloromethane and washed with NaOH 1 M aqueous solution, water and brine. The organic layer was dried (MgSO4) and concentrated in vacuo. The residue was purified by chromatography on silica eluting with 0-50% ethyl acetate in iso-hexane to give the title compound as a pale pink oil (0.303 g). ¹H NMR (400 MHz, CDCl₃) δ 5.80 (br d, J=6.3 Hz, 1H), 4.38-4.29 (m, 1H), 4.19 (q, J=7.1 Hz, 2H), 3.60 (s, 2H), 1.30-1.25 (m, 9H); LCMS (Method 3) Rt 1.48 min m/z 292-294 [M+H]⁺.

### Intermediate 32: 2-(2,4-Dichloro-6-(isopropylamino)pyrimidin-5-yl)ethan-1-ol

Intermediate 31 (0.5 g) was dissolved in THF (5.0 mL) and then LiAlH₄ (1.0 mL) was added dropwise at 0-5° C. and the resulting mixture was stirred at room temperature for 1 hour. The reaction mixture was cooled to 0-5° C. and quenched by addition of Rochelle salt saturated solution. The mixture was allowed to stand overnight. The mixture was diluted with dichloromethane, filtered through Celite and the organic layer was separated and concentrated in vacuo. The residue was purified by chromatography on silica eluting with 0-100% ethyl acetate in iso-hexane to give the title compound as a white solid (57 mg). ¹H NMR (400 MHz, CDCl₃) δ 6.20 (s, 1H), 4.29 (sept, J=6.6 Hz, 1H), 3.95 (app q, J=5.5 Hz, 2H), 2.82 (t, J=5.5 Hz, 2H), 1.93 (br t, J=3.8 Hz, 1H), 1.22 (d, J=6.6 Hz, 6H); LCMS (Method 3) Rt 1.21 min m/z 250-252-254 [M+H]⁺.

### Intermediate 33: 2-(2,4-Dichloro-6-(isopropylamino)pyrimidin-5-yl)ethyl methanesulfonate

Methanesulfonyl chloride (18 μL) was added to a stirred solution of intermediate 32 (57 mg), and TEA (32 μL) in dichloromethane and the resulting mixture was allowed to stir at room temperature for 1 hour. The solvent was removed in vacuo and the residue was dissolved in dichloromethane and washed with water and brine. The organic layer was dried (MgSO₄) and concentrated in vacuo to afford the title compound as a solid (63.2 mg). ¹H NMR (400 MHz, CDCl₃) δ 5.61 (br d, J=7.0 Hz, 1H), 4.38 (t, J=7.0 Hz, 3H), 3.05-3.03 (m, 5H), 1.26 (d, J=6.1 Hz, 6H); LCMS (Method 3) Rt 1.49 min m/z 328-329-331 [M+H]⁺.

### Intermediate 34: 2,4-Dichloro-7-isopropyl-6,7-dihydro-5H-pyrrolo[2,3-d]pyrimidine

Intermediate 33 (42 mg) was dissolved in acetonitrile (1.0 mL) and then sodium hydride (60%) (6.8 mg) was added in one portion and the resulting mixture was stirred for 1 hour. The mixture was diluted with ethyl acetate and washed with water. The organic layer was separated, dried (MgSO₄) and concentrated in vacuo to afford a brown oil. The residue was purified by chromatography on silica eluting with 0-50% ethyl acetate in iso-hexane to give the title compound as a white solid (33 mg). ¹H NMR (400 MHz, CDCl₃) δ 4.41 (sept, J=6.2 Hz, 1H), 3.68 (t, J=8.0 Hz, 2H), 3.01 (t, J=8.0 Hz, 2H), 1.21 (d, J=6.2 Hz, 6H); LCMS (Method 3) Rt 1.33 min m/z 232/234/236 [M+H]⁺.

### Intermediate 35: 4-(2-((2-Chloro-7-isopropyl-6,7-dihydro-5H-pyrrolo[2,3-d]pyrimidin-4-yl)amino)ethyl)phenol

Intermediate 34 (95 mg), tyramine (62 mg) and 2-propanol (3.0 mL) were stirred at 50° C. for 3 hours. Further tyramine (62 mg) was added and the mixture was heated overnight at 90° C. for 48 hours. The reaction mixture was cooled to room temperature and concentrated in vacuo. The residue was purified by chromatography on silica eluting with 0-50% ethyl acetate in iso-hexane to give the title compound as a pale yellow foam (79 mg). ¹H NMR (400 MHz, CDCl₃) δ 7.04 (d, J=8.4 Hz, 2H), 6.74 (d, J=8.4 Hz, 2H), 5.90 (br s, 1H), 4.86 (br s, 1H), 4.38-4.26 (m, 1H), 3.60-3.48 (m, 4H), 2.88 (t, J=8.4 Hz, 2H), 2.79 (t, J=7.3 Hz, 2H), 1.19 (d, J=7.8 Hz, 6H); LCMS (Method 3) Rt 1.10 min m/z 333 [M+H]⁺.

### Example 18: 4-(2-((2-(Benzo[b]thiophen-3-yl)-7-isopropyl-6,7-dihydro-5H-pyrrolo[2,3-d]pyrimidin-4-yl)amino)ethyl)phenol

Intermediate 35 (79 mg), benzo[b]thiophen-3-ylboronic acid (50 mg), tetrakis-(triphenylphosphine)palladium (0) (27.44 mg), cesium carbonate (231 mg) were suspended in dioxane (3.0 mL) and water (2.0 mL) and the resulting mixture was degassed under argon and then heated at 85° C. under microwave irradiation for 1 hour. The reaction mixture was further irradiated at 120° C. for 1 hour and then at 100° C. for 4 hours. The mixture was concentrated in vacuo and diluted with ethyl acetate. The organic phase was washed with water and brine, then dried (MgSO₄) and concentrated in vacuo. The residue was purified by chromatography on silica eluting with 0-100% ethyl acetate in iso-hexane to give the semicrude product as a brown oil (72 mg). The residue was purified by HPLC (Kinetix C18 RP column, 5-98% CH₃CN/H₂O [0.1% NH₄OH] @18 mL/min over 20 min gradient) and freeze-dried to afford the title compound as a pale yellow solid (16 mg). ¹H NMR (400 MHz, d6-DMSO) δ 9.14 (s, 1H), 8.72 (br d, J=7.2 Hz, 1H), 8.04-7.99 (m, 2H), 7.43-7.34 (m, 2H), 7.02 (d, J=8.5 Hz, 2H), 6.67 (d, J=8.5 Hz, 2H), 6.48 (app t, J=5.7 Hz, 1H), 4.32 (sept, J=6.7 Hz, 1H), 3.52 (t, J=7.7 Hz, 2H), 3.43 (app q, J=7.4 Hz, 2H), 3.03 (t, J=7.4 Hz, 2H), 2.75 (t, J=7.7 Hz, 2H), 1.17 (d, J=6.7 Hz, 6H); LCMS (Method 1) Rt 3.94 min m/z 431 [M+H]⁺.

### Compound Example 19: 2-(Benzo[b]thiophen-3-yl)-4-((4-hydroxyphenethyl)amino)-7-isopropyl-5,7-dihydro-6H-pyrrolo[2,3-d]pyrimidin-6-one

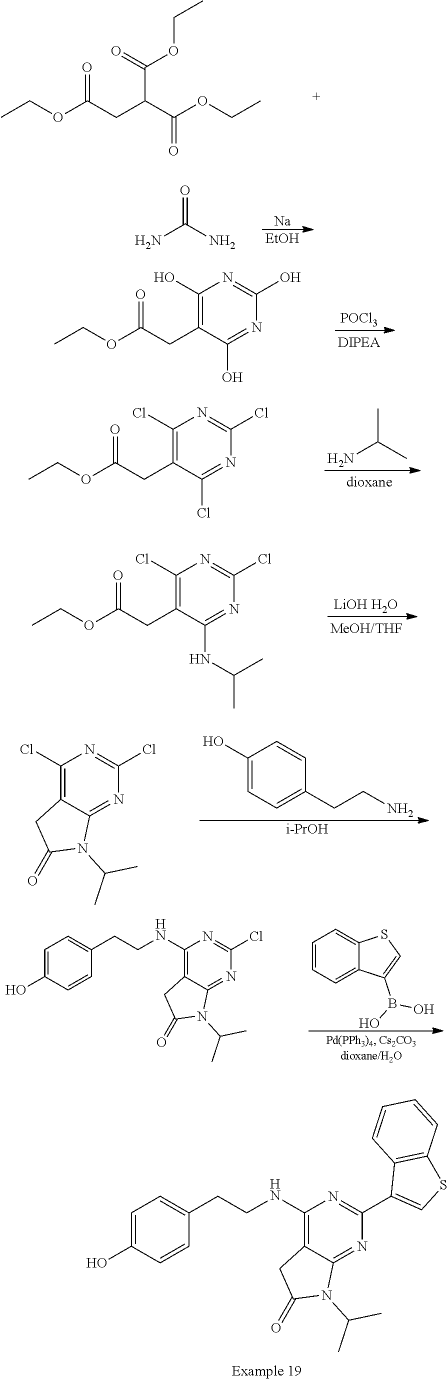

### Intermediate 36: 2,4-Dichloro-7-isopropyl-5,7-dihydro-6H-pyrrolo[2,3-d]pyrimidin-6-one

A solution of intermediate 31 (0.20 g) in THF (3.0 mL) and methanol (1.0 mL) was treated with lithium hydroxide monohydrate 2 M aqueous solution (1.0 mL) and the resulting mixture was allowed to stir at room temperature for 3 hours. The reaction mixture was concentrated in vacuo and the residue was partitioned between ethyl acetate and water and neutralised with HCl 1M aqueous solution. The organic layer was separated and dried to afford a brown oil. The residue was purified by chromatography on silica eluting with 0-50% ethyl acetate in iso-hexane to give the title compound as a white solid (95 mg). ¹H NMR (400 MHz, CDCl₃) δ 4.66 (sept, J=7.1 Hz, 1H), 3.54 (s, 2H), 1.51 (d, J=7.1 Hz, 6H); LCMS (Method 3) Rt 1.46 min m/z 246/248/250 [M+H]⁺.

### Intermediate 37: 2-Chloro-4-((4-hydroxyphenethyl)amino)-7-isopropyl-5,7-dihydro-6H-pyrrolo[2,3-d]pyrimidin-6-one

Intermediate 36 (0.124 g) and tyramine (0.076 g) were dissolved in 2-propanol (5.0 mL) and the resulting mixture was stirred to reflux for 72 hours. The cooled mixture was concentrated in vacuo. The residue was purified by chromatography on silica eluting with 0-100% ethyl acetate in iso-hexane to give the title compound as a light brown solid (69 mg). ¹H NMR (400 MHz, CDCl₃) δ 7.09 (d, J=8.4 Hz, 2H), 6.78 (d, J=8.4 Hz, 2H), 5.21 (br s, 1H), 5.06 (br s, 1H), 4.65-4.52 (br m, 1H), 3.63 (app q, J=6.9 Hz, 2H), 3.40 (s, 2H), 2.84 (t, J=6.9 Hz, 2H), 1.50 (d, J=6.8 Hz, 6H).

### Compound Example 19: 2-(Benzo[b]thiophen-3-yl)-4-((4-hydroxyphenethyl)amino)-7-isopropyl-5,7-dihydro-6H-pyrrolo[2,3-d]pyrimidin-6-one

Intermediate 37 (38 mg), benzo[b]thien-3-ylboronic acid (23 mg), tetrakis-(triphenylphosphine)palladium (0) (13 mg), cesium carbonate (106 mg) were suspended in dioxane (2.0 mL) and water (0.8 mL) and the resulting mixture was degassed under argon and then heated at 90° C. under microwave irradiation for 1.5 hour. The mixture was concentrated in vacuo and diluted with ethyl acetate. The organic phase was washed with water and brine, then dried (MgSO₄) and concentrated in vacuo. The residue was purified by chromatography on silica eluting with 0-100% ethyl acetate in iso-hexane to give the semicrude product. The residue was purified by HPLC (Basic, Kinetix C18 RP column, 30-98% CH₃CN/H₂O [0.1% NH₄OH] @18 mL/min over 20 min gradient) and freeze-dried to afford the title compound as a pale yellow solid (33 mg). ¹H NMR (400 MHz, d6-DMSO) δ 9.16 (s, 1H), 8.79 (d, J=8.5 Hz, 1H), 8.28 (s, 1H), 8.07 (d, J=8.5 Hz, 1H), 7.47-7.40 (m, 2H), 7.27 (br s, 1H), 7.04 (d, J=8.6 Hz, 2H), 6.68 (d, J=8.6 Hz, 2H), 4.62-4.52 (br m, 1H), 3.72 (s, 2H), 3.59-3.46 (br m, 2H), 2.81 (t, J=8.1 Hz, 2H), 1.48 (br s, 6H); LCMS (Method 1) Rt 4.47 min m/z 445 [M+H]⁺.

### Compound Example 20: 4-(2-((2-(Benzo[b]thiophen-3-yl)-8-(dimethylamino)pyrimido[5,4-d]pyrimidin-4-yl)amino)ethyl)phenol

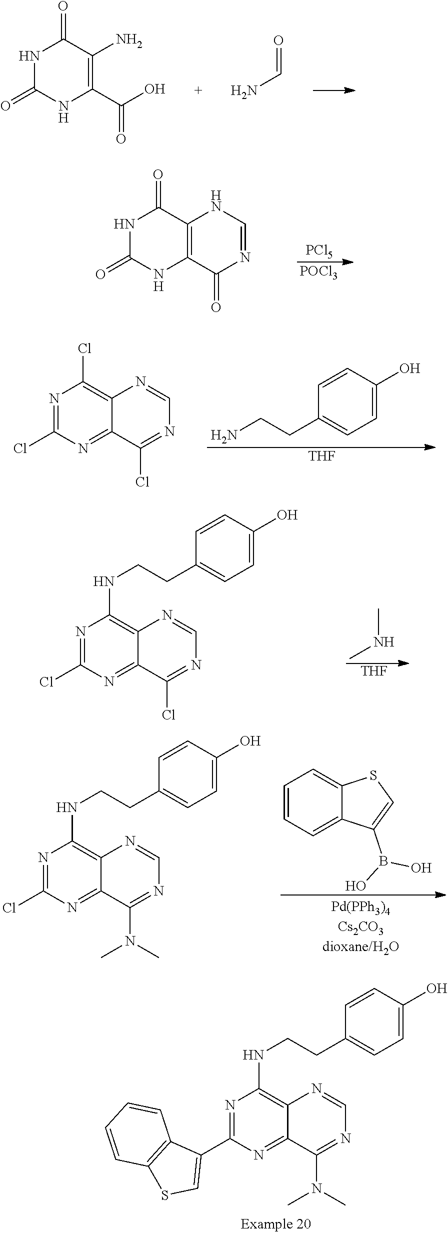

### Intermediate 38: 1,5-Dihydropyrimido[5,4-d]pyrimidine-2,4,8(3H)-trione

5-Aminoorotic acid (10.0 g) and formamide (100 mL) were stirred at 170° C. overnight. The mixture was cooled to room temperature for 2 hours and the resulting precipitate was isolated by filtration and washed with IMS. The solid was dried at 40° C. in vacuo overnight to afford the title compound as a light brown solid (6.73 g). NMR (400 MHz, d6-DMSO) δ 11.57 (s, 1H), 11.08 (s, 1H), 7.95 (s, 1H).

### Intermediate 39: 2,4,8-Trichloropyrimido[5,4-d]pyrimidine

Intermediate 38 (2.5 g) was dissolved in phosphorus(V) oxychloride (100 mL) and phosphorus pentachloride (12.5 g) was added and the resulting mixture was stirred at room temperature and then at reflux for 5 hours. The reaction mixture was cooled to room temperature and stirred for 48 hours. The mixture was concentrated in vacuo, then it was diluted with iced water (100 mL) and vigorously stirred for 30 minutes. The resulting precipitate was isolated by filtration and dried in vacuo overnight to afford the title compound as a light brown solid (2.15 g). ¹H NMR (400 MHz, CDCl₃) δ 9.29 (s, 1H).

### Intermediate 40: 4-(2-((2,8-Dichloropyrimido[5,4-d]pyrimidin-4-yl)amino)ethyl)phenol

Intermediate 39 (0.40 g) was dissolved in THF (10 mL) and the solution was cooled to 0-5° C. under argon atmosphere. Then a suspension of tyramine (0.213 g) in THF (5 mL) was added and the resulting mixture was allowed to stir for 1 hour at 0-5° C. The reaction mixture was concentrated in vacuo and the solid residue was diluted with dichloromethane and washed with NaHCO₃ aqueous saturated solution, water and brine. The organic layer was dried (MgSO4) and concentrated in vacuo. The residue was purified by chromatography on silica eluting with 0-10% methanol in dichloromethane to give the title compound as a yellow solid (0.36 g). ¹H NMR (400 MHz, CDCl₃) δ 9.25 (s, 1H), 8.88 (s, 1H), 7.34 (s, 1H), 7.13 (d, J=7.9 Hz, 2H), 6.81 (d, J=7.9 Hz, 2H), 3.91 (app q, J=7.0 Hz, 2H), 2.97 (t, J=7.0 Hz, 2H).

### Intermediate 41: 4-(2-((2-Chloro-8-(dimethylamino)pyrimido[5,4-d]pyrimidin-4-yl)amino)ethyl)phenol

Intermediate 40 (0.15 g) was dissolved in THF (5 mL) then a 2M solution of dimethylamine (225 μL) was added and the resulting mixture was allowed to stir at room temperature overnight. The reaction mixture was concentrated in vacuo and the solid residue was purified by chromatography on silica eluting with 0-100% ethyl acetate in iso-hexane to give the title compound as a yellow gum (36 mg). LCMS (Method 3) Rt 1.45 min m/z 345/347 [M+H]⁺.

### Example 20: 4-(2-((2-(Benzo[b]thiophen-3-yl)-8-(dimethylamino)pyrimido[5,4-d]pyrimidin-4-yl)amino)ethyl)phenol

Intermediate 41 (75 mg), benzo[b] thien-3-ylboronic acid (46.3 mg), tetrakis-(triphenylphosphine)palladium (0) (25.3 mg), cesium carbonate (212.5 mg) were suspended in dioxane (3.5 mL) and water (1.65 mL) and the resulting mixture was degassed under argon and heated at 120° C. under microwave irradiation for 1 hour. The cooled mixture was concentrated in vacuo and diluted with ethyl acetate. The organic phase was washed with water and brine, then dried (MgSO₄) and concentrated in vacuo. The residue was purified by chromatography on silica eluting with 0-100% ethyl acetate in iso-hexane to give the semicrude product. The residue was purified by HPLC (Acid, Kinetix C18 RP column, 40-98% CH₃CN/H₂O [0.1% HCO₂H] @18 mL/min over 5 min gradient) and freeze-dried to afford the title compound as a white solid (7.5 mg). ¹H NMR (400 MHz, d6-DMSO) δ 9.22 (s, 1H), 9.09-9.05 (m, 1H), 8.61 (s, 1H), 8.45 (s, 1H), 8.30 (app t, J=6.1 Hz, 1H), 8.10-8.06 (m, 1H), 7.49-7.43 (m, 2H), 7.13 (d, J=8.4 Hz, 2H), 6.71 (d, J=8.4 Hz, 2H), 3.82 (app q, J=7.0, 2H), 3.33 (br s, 6H), 2.92 (t, J=7.0 Hz, 2H); LCMS (Method 1) Rt 5.97 min m/z 443 [M+H]⁺.

### Compound Example 21: N-(2-(1H-Indol-3-yl)ethyl)-2-methyl-6-phenylthieno[2,3-d]pyrimidin-4-amine

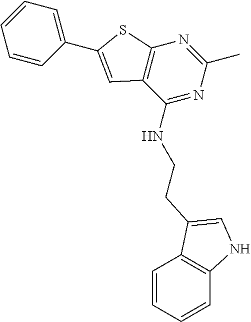 [SMILES: Cc1nc(NCCc2c[nH]c3ccccc23)c2cc(-c3ccccc3)sc2n1]

45 mg of the commercial compound (Enamine Z2239048492, CAS 565166-55-4) was purified by MDAP (acidic method) to afford the title compound as a white solid (33.5 mg). ¹H NMR (400 MHz, d6-DMSO) δ 10.82 (s, 1H), 8.05-8.01 (m, 1H), 7.96 (s, 1H), 7.71-7.64 (m, 3H), 7.49 (t, J=8.2 Hz, 2H), 7.37 (dd, J=7.7, 15.6 Hz, 2H), 7.21 (d, J=2.3 Hz, 1H), 7.10-6.97 (m, 2H), 3.78 (dd, J=6.3, 14.4 Hz, 2H), 3.05 (t, J=7.5 Hz, 2H), 2.51 (s, 3H); LCMS (Method 1) Rt 4.43 min m/z 385 [M+H]⁺.

### Compound Example 22 (ADS160850): N-(2-(1H-Indol-3-yl)ethyl)-6-(4-fluorophenyl)thieno[3,2-d]pyrimidin-4-amine

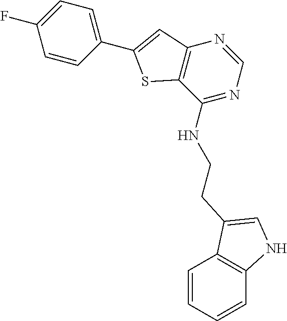 [SMILES: Fc1ccc(-c2cc3ncnc(NCCc4c[nH]c5ccccc45)c3s2)cc1]

45 mg of the commercial compound (Enamine Z2239063077, CAS 878243-73-3) was purified by MDAP (acidic method) to afford the title compound as a pale yellow solid (24 mg). ¹H NMR (400 MHz, DMSO) δ 10.81 (s, 1H), 8.49 (s, 1H), 8.03 (app t, J=5.3 Hz, 1H), 7.90 (ddd, J=3.1, 5.3, 12.0 Hz, 2H), 7.80 (s, 1H), 7.62 (d, J=7.8 Hz, 1H), 7.40-7.33 (m, 3H), 7.20 (d, J=2.2 Hz, 1H), 7.10-6.97 (m, 2H), 3.83-3.75 (m, 2H), 3.09-3.02 (m, 2H); LCMS (Method 1) Rt 3.72 min m/z 389 [M+H]⁺.

### Example 2

### Expansion of CD34+ Hematopoietic Stem Cells

The compounds above were evaluated for their ability to promote expansion/proliferation in hematopoietic stem cell cultures. Specifically, umbilical cord blood CD34+ cells which were isolated by antibody-based cell sorting (StemCell Technology) were thawed and expanded in vitro as follows.

CD34+ cells are cultured in the following medium formulations, and aliquots of cells are taken for assessment of cell count, cell viability.

Stage 1 medium: 90% Stem Cell Growth Medium (SCGM) (CellGro®), 10% Human Serum-AB, supplemented with 25 ng/mL recombinant human thrombopoietin (TPO), 25 ng/mL recombinant human Flt3L, 27 ng/mL recombinant human stem cell factor (SCF), 25 ng/mL recombinant human IL-7, 0.05 ng/mL recombinant human IL-6 (500-fold), 0.25 ng/mL recombinant human granulocyte colony-stimulating factor (G-CSF) (50-fold), 0.01 ng/mL recombinant human granulocyte-macrophage colony-stimulating factor (GM-CSF) (500-fold), and 0.10% gentamicin.

Stage 2 medium: 90% SCGM, 10% Human Serum-AB, supplemented with 25 ng/mL recombinant human Flt3L, 27 ng/mL recombinant human SCF, 25 ng/mL recombinant human IL-7, 20 ng/mL recombinant human IL-15, 0.05 ng/mL recombinant human IL-6 (500-fold), 0.25 ng/mL recombinant human G-CSF (50-fold), 0.01 ng/mL recombinant human GM-CSF (500-fold), and 0.10% gentamicin.

Cells were maintained in log phase by addition of Stage 1 medium from day 0 to day 9 and by addition of Stage 2 medium from day 10 to day 14. At day 14 FACS cell counting and analysis was performed to determine expansion of hematopoietic stem cells.

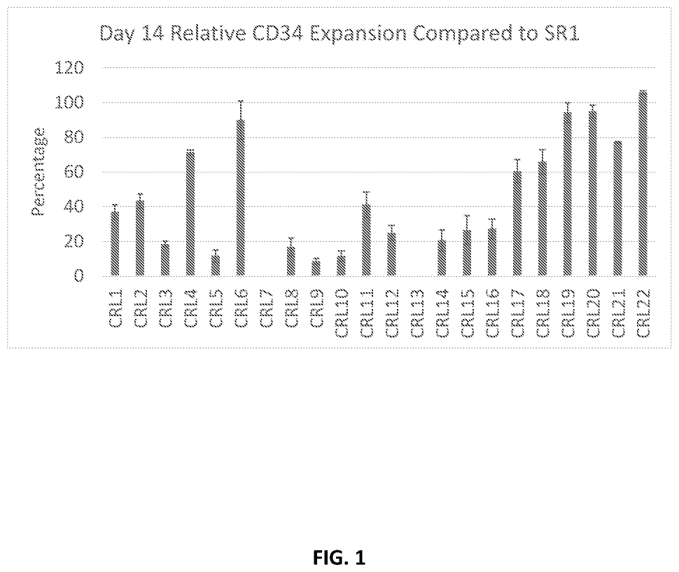

During the 14 days of the culture, each CRL compound was dissolved in DMSO and added to the culture at 10 μM concentration. Because previous studies have shown that Stemregenin 1 (SR1) is a known commercial reagent for hematopoietic stem cell expansion, SR1 (at 10 μM) served as a positive control compound, while DMSO alone without any compound served as a negative control. Results are representative of several experiments and are normalized to the positive control for comparison purposes. The DMSO negative control, resulted in an expansion of 15 20% of that of SR1. Thus, FIG. 1 shows robust expansion of CD34+ hematopoietic stem cells for about half of the 22 compounds tested for the family of compounds discovered indicating significant utility for these compounds in the expansion and proliferation of stem cells, hematopoietic stem cells and progenitor cells.

In the subject experiments, hematopoietic stem cells were being expanded toward the natural killer cell lineage. Increases in cell numbers were seen throughout the expansion, suggesting that the compounds of the invention served to expand not just hematopoietic stem cells, but progenitor cells that had begun to differentiate towards a desired lineage. Based on these results, it is believed that the compounds of the invention are useful for the expansion of stem cells, the expansion of progenitor cells, and the expansion of differentiated cells which result from the further expansion/differentiation of such cells.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

1. A compound having the structure of the Formula (I): 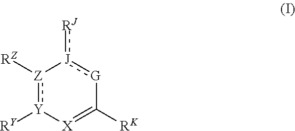
including pharmaceutically acceptable salts thereof, wherein:
each [symbol] independently represents a single bond or a double bond;
R^{J} is selected from the group consisting of —NR^{a}R^{b}, —OR^{b}, and ═O, wherein if R^{J} is ═O, then [symbol] joining G and J represents a single bond and G is N and the N is substituted with R^{G}; otherwise [symbol] joining G and J represents a double bond and G is N;
R^{a} is hydrogen or C₁-C₄ alkyl;
R^{b} is R^{c} or —(C₁-C₄ alkyl)-R^{c};
R^{c} is selected from the group consisting of: —OH, —O(C₁-C₄ alkyl), —O(C₁-C₄ haloalkyl); —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E is independently selected from the group consisting of: —OH, C₁-C₄ alkyl, C₁-C₄ haloalkyl, —O(C₁-C₄ alkyl), and —O(C₁-C₄ haloalkyl);
R^{K} is selected from the group consisting of: hydrogen, unsubstituted C₁₋₆ alkyl; substituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q is independently selected from the group consisting of: —OH, C₁₋₄ alkyl, C₁₋₄ haloalkyl, halo, cyano, —O—(C₁₋₄ alkyl), and —O—(C₁₋₄ haloalkyl);
R^{G} is selected from the group consisting of hydrogen, C₁₋₄ alkyl, and —(C₁₋₄ alkyl)-C(═O)NH₂;
R^{Y} and R^{Z} are each independently absent or selected from the group consisting of: hydrogen, halo, C₁₋₆ alkyl, —OH, —O—(C₁₋₄ alkyl), —NH(C₁₋₄ alkyl), and —N(C₁₋₄ alkyl)₂;
or R^{Y} and R^{Z} taken together with the atoms to which they are attached are joined together to form a ring selected from: 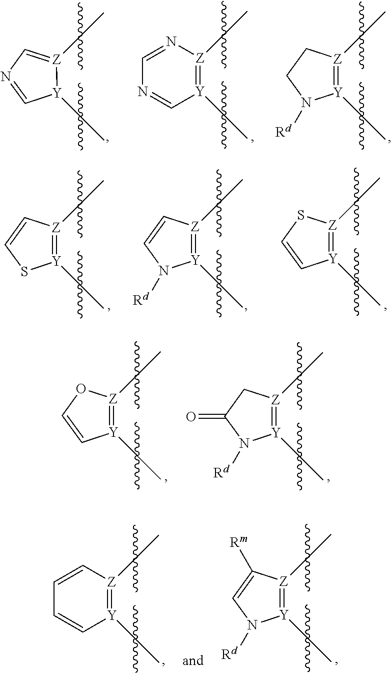
wherein said ring is optionally substituted with one, two, or three groups independently selected from C₁₋₄ alkyl, C₁₋₄ haloalkyl, halo, cyano, —OH, —O—(C₁₋₄ alkyl), —N(C₁₋₄ alkyl)₂, unsubstituted C₆-C₁₀ aryl, C₆-C₁₀ aryl substituted with 1-5 halo atoms, and —O—(C₁₋₄ haloalkyl); and wherein if R^{Y} and R^{Z} taken together forms 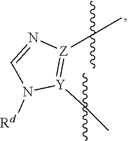
then R^{J} is —OR^{b} or ═O;
R^{d} is hydrogen or C₁-C₄ alkyl;
R^{m} is selected from the group consisting of C₁₋₄ alkyl, halo, and cyano;
J is C; and
X, Y, and Z are each independently N or C, wherein the valency of any carbon atom is filled as needed with hydrogen atoms.

2. The compound of claim 1, wherein:
R^{a} is hydrogen;
R^{b} is —(C₁-C₄ alkyl)-R^{c};
R^{c} is selected from the group consisting of: —C(═O)NH₂; unsubstituted C₆₋₁₀ aryl;
substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E is independently selected from the group consisting of: —OH, C₁-C₄ alkyl, C₁-C₄ haloalkyl, —O(C₁-C₄ alkyl), and —O(C₁-C₄ haloalkyl);
R^{K} is selected from the group consisting of: hydrogen, unsubstituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five-to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q is independently selected from the group consisting of: —OH, C₁₋₄ alkyl, C₁₋₄ haloalkyl, halo, cyano, —O—(C₁₋₄ alkyl), and —O—(C₁₋₄ haloalkyl);
R^{G} is —(C₁₋₄ alkyl)-C(═O)NH₂;
R^{Y} and R^{Z} are each independently absent or selected from the group consisting of: hydrogen, C₁₋₆ alkyl, and —NH(C₁₋₄ alkyl);
or R^{Y} and R^{Z} taken together with the atoms to which they are attached are joined together to form a ring selected from: 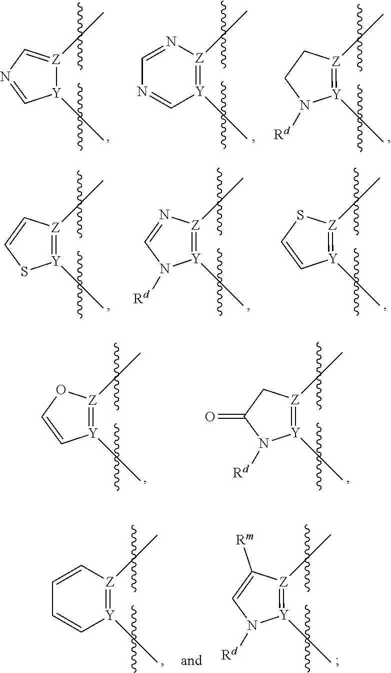
wherein said ring is optionally substituted with one, two, or three groups independently selected from C₁₋₄ alkyl, C₁₋₄ haloalkyl, halo, cyano, —OH, —O—(C₁₋₄ alkyl), —N(C₁₋₄ alkyl)₂, unsubstituted C₆-C₁₀ aryl, C₆-C₁₀ aryl substituted with 1-5 halo atoms, and —O—(C₁₋₄ haloalkyl);
R^{d} is C₁-C₄ alkyl;
R^{m} is cyano;
and
X, Y, and Z are each independently N or C, wherein the valency of any carbon atom is filled as needed with hydrogen atoms.

3. The compound of claim 1, wherein:
R^{a} is hydrogen;
R^{b} is —CH₂CH₂—R^{c};
R^{c} is selected from the group consisting of: unsubstituted phenyl, substituted phenyl, indolyl, and —C(═O)NH₂;
R^{K} is selected from the group consisting of: hydrogen, methyl, substituted pyridinyl, unsubstituted benzothiophenyl, and —NH(C₁-C₄ alkyl);
R^{G} is —CH₂CH₂—C(═O)NH₂;
R^{Y} is —NH(C₁-C₄ alkyl);
R^{Z} is absent or hydrogen;
or R^{Y} and R^{Z} taken together with the atoms to which they are attached are joined together to form a ring selected from: 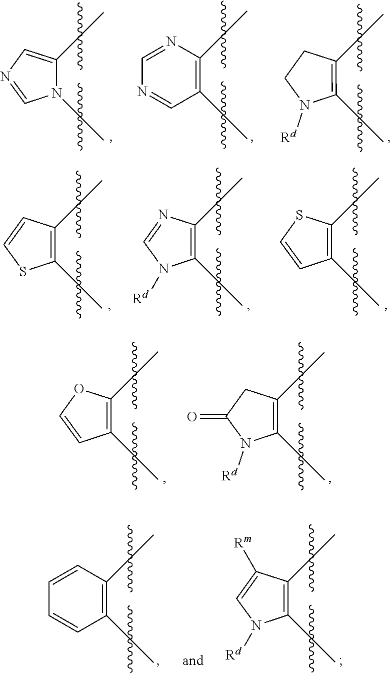
wherein said ring is optionally substituted with one, two, or three groups independently selected from C₁-C₄ alkyl, —N(C₁-C₄ alkyl)₂, cyano, unsubstituted phenyl, and phenyl substituted with 1-5 halo atoms;
R^{d} is C₁-C₄ alkyl;
R^{m} is cyano; and
X is N or CH.

4. The compound of claim 1, wherein:
R^{a} is hydrogen;
R^{b} is —CH₂CH₂—R^{c};
R^{c} is selected from the group consisting of: unsubstituted phenyl, substituted phenyl, indolyl, and —C(═O)NH₂; wherein the substituted phenyl is substituted with one substituent E, wherein E is —OH;
R^{K} is selected from the group consisting of: hydrogen, methyl, substituted pyridinyl, unsubstituted benzothiophenyl, and —NH(sec-butyl); wherein the substituted pyridinyl moiety is substituted with one substituent Q, wherein Q is selected from the group consisting of: C₁₋₄ alkyl, halo, and cyano;
R^{G} is —CH₂CH₂—C(═O)NH₂;
R^{Y} is —NH(isopropyl) or —NH(sec-butyl);
R^{Z} is absent or hydrogen;
or R^{Y} and R^{Z} taken together with the atoms to which they are attached are joined together to form a ring selected from: 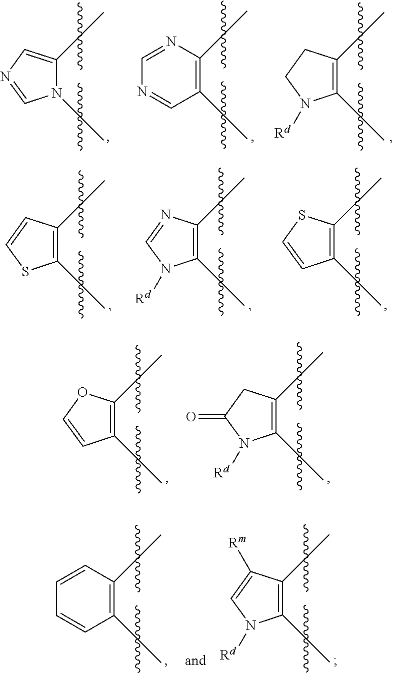
wherein said ring is optionally substituted with one, two, or three groups independently selected from C₁-C₄ alkyl, cyano, unsubstituted phenyl, and 4-fluorophenyl;
R^{d} is isopropyl;
R^{m} is cyano; and
X is N or CH.

5. The compound of claim 1, wherein the compound of Formula (I) has the structure of Formula (I-A): 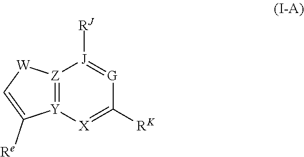
including pharmaceutically acceptable salts thereof, wherein:
R^{J} is —NR^{a}R^{b};
R^{a} is hydrogen or C₁-C₄ alkyl;
R^{b} is R^{c} or —(C₁-C₄alkyl)-R^{c};
R^{c} is selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E is independently selected from the group consisting of: —OH, C₁-C₄ alkyl, C₁-C₄ haloalkyl, —O(C₁-C₄ alkyl), and —O(C₁-C₄ haloalkyl);
R^{K} is selected from the group consisting of: hydrogen, unsubstituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five-to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q is independently selected from the group consisting of: —OH, C₁₋₄ alkyl, C₁₋₄ haloalkyl, halo, cyano, —O—(C₁₋₄ alkyl), and —O—(C₁₋₄ haloalkyl);
Y and Z are each C;
X is N or CH;
W is O or S; and
R^{e} is hydrogen or C₁-C₄ alkyl.

6. The compound of claim 5, wherein:
R^{a} is hydrogen;
R^{b} is —(C₁-C₄alkyl)-R^{c};
R^{c} is selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E is independently selected from the group consisting of: —OH, C₁-C₄ alkyl, C₁-C₄ haloalkyl, —O(C₁-C₄ alkyl), and —O(C₁-C₄ haloalkyl);
R^{K} is selected from the group consisting of: unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein the substituted heteroaryl is substituted with one or more substituents Q, wherein each Q is independently selected from the group consisting of: —OH, C₁₋₄ alkyl, C₁₋₄ haloalkyl, halo, cyano, —O—(C₁₋₄ alkyl), and —O—(C₁₋₄ haloalkyl); and
R^{e} is C₁-C₄ alkyl.

7. The compound of claim 5, wherein:
R^{a} is hydrogen;
R^{b} is —(CH₂—CH₂)—R^{c};
R^{c} is selected from the group consisting of: substituted phenyl and unsubstituted indolyl; wherein the substituted phenyl is substituted with one substituent E, wherein E is —OH;
R^{K} is selected from the group consisting of: unsubstituted benzothiophenyl and substituted pyridinyl; wherein the substituted pyridinyl is substituted with one substituent Q, wherein Q is selected from the group consisting of: C₁₋₄ alkyl, halo, and cyano; and
R^{e} is isopropyl.

8. (canceled)

9. The compound of claim 1, wherein the compound of Formula (I) has the structure of Formula (I-B): 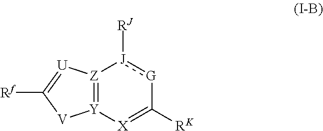
including pharmaceutically acceptable salts thereof, wherein:
R^{a} is hydrogen or C₁-C₄ alkyl;
R^{b} is R^{c} or —(C₁₋₄ alkyl)-R^{c};
R^{c} is selected from the group consisting of: —OH, —O(C₁-C₄ alkyl), —O(C₁-C₄ haloalkyl); —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E is independently selected from the group consisting of: —OH, C₁-C₄ alkyl, C₁-C₄ haloalkyl, —O(C₁-C₄ alkyl), and —O(C₁-C₄ haloalkyl);
R^{K} is selected from the group consisting of: hydrogen, unsubstituted C₁₋₆ alkyl; substituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q is independently selected from the group consisting of: —OH, C₁₋₄ alkyl, C₁₋₄ haloalkyl, halo, cyano, —O—(C₁₋₄ alkyl), and —O—(C₁₋₄ haloalkyl);
R^{G} is selected from the group consisting of hydrogen, C₁₋₄ alkyl, and —(C₁₋₄ alkyl)-C(═O)NH₂;
R^{f} is selected from the group consisting of hydrogen, C₁₋₄ alkyl, unsubstituted C₆-C₁₀ aryl, and C₆-C₁₀ aryl substituted with 1-5 halo atoms;
U is N or CR^{U};
V is S or NR^{V};
R^{U} is selected from the group consisting of hydrogen, C₁₋₄ alkyl, halo, and cyano;
R^{V} is hydrogen or C₁-C₄ alkyl;
wherein when U is CR^{U} and V is NR^{V}, R^{U} is selected from the group consisting of C₁₋₄ alkyl, halo, and cyano;
Y and Z are each C; and
X is N or CH.

10. The compound of claim 9, wherein:
R^{a} is hydrogen;
R^{b} is —(C₁₋₄ alkyl)-R^{c};
R^{c} is selected from the group consisting of: —C(═O)NH₂, unsubstituted C₆₋₁₀ aryl;
substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E is independently selected from the group consisting of: —OH, C₁-C₄ alkyl, C₁-C₄ haloalkyl, —O(C₁-C₄ alkyl), and —O(C₁-C₄ haloalkyl);
R^{K} is selected from the group consisting of: unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein the substituted heteroaryl is substituted with one or more substituents Q, wherein each Q is independently selected from the group consisting of: —OH, C₁₋₄ alkyl, C₁₋₄ haloalkyl, halo, cyano, —O—(C₁₋₄ alkyl), and —O—(C₁₋₄ haloalkyl);
R^{G} is C₁₋₄ alkyl or —(C₁₋₄ alkyl)-C(═O)NH₂;
R^{f} is selected from the group consisting of hydrogen, unsubstituted phenyl, and phenyl substituted with 1-5 halo atoms;
Y and Z are each C; and
X is CH.

11. The compound of claim 9, wherein:
R^{a} is hydrogen;
R^{b} is —(CH₂—CH₂)—R^{c};
R^{c} is selected from the group consisting of: —C(═O)NH₂, substituted phenyl and unsubstituted indolyl; wherein the substituted phenyl is substituted with one substituent E, wherein E is —OH;
R^{K} is selected from the group consisting of: unsubstituted benzothiohenyl and substituted pyridinyl; wherein the substituted pyridinyl is substituted with one substituent Q, wherein Q is selected from the group consisting of: C₁₋₄ alkyl, halo, and cyano;
R^{G} is —(CH₂CH₂)—C(═O)NH₂;
R^{f} is selected from the group consisting of hydrogen, phenyl, and fluorophenyl;
Y and Z are each C; and
X is CH.

12. (canceled)

13. The compound of claim 1, wherein the compound of Formula (I) has the structure of Formula (I-C): 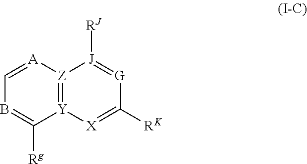
including pharmaceutically acceptable salts thereof, wherein:
R^{J} is —NR^{a}R^{b};
R^{a} is hydrogen or C₁-C₄ alkyl;
R^{b} is R^{c} or —(C₁-C₄ alkyl)-R^{c};
R^{c} is selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E is independently selected from the group consisting of: —OH, C₁-C₄ alkyl, C₁-C₄ haloalkyl, —O(C₁-C₄ alkyl), and —O(C₁-C₄ haloalkyl);
R^{K} is selected from the group consisting of: hydrogen, unsubstituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five-to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q is independently selected from the group consisting of: —OH, C₁₋₄ alkyl, C₁₋₄ haloalkyl, halo, cyano, —O—(C₁₋₄ alkyl), and —O—(C₁₋₄ haloalkyl);
A is N or CH;
B is N or CH;
R^{g} is selected from the group consisting of hydrogen, C₁₋₄ alkyl, and —N(C₁₋₄ alkyl)₂;
Y and Z are each C; and
X is N or CH.

14. The compound of claim 13, wherein:
R^{a} is hydrogen;
R^{b} is —(C₁-C₄alkyl)-R^{c};
R^{c} is selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E is independently selected from the group consisting of: —OH, C₁-C₄ alkyl, C₁-C₄ haloalkyl, —O(C₁-C₄ alkyl), and —O(C₁-C₄ haloalkyl);
R^{K} is selected from the group consisting of: —NH(C₁₋₄ alkyl); unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein the substituted heteroaryl is substituted with one or more substituents Q, wherein each Q is independently selected from the group consisting of: —OH, C₁₋₄ alkyl, C₁₋₄ haloalkyl, halo, cyano, —O—(C₁₋₄ alkyl), and —O—(C₁₋₄ haloalkyl); and
R^{g} is hydrogen or —N(C₁₋₄ alkyl)₂.

15. The compound of claim 13, wherein:
R^{a} is hydrogen;
R^{b} is —(C₁-C₄ alkyl)-R^{c};
R^{c} is selected from the group consisting of: substituted phenyl and unsubstituted indolyl; wherein the substituted phenyl is substituted with one or more substituents E, wherein each E is independently selected from the group consisting of: —OH, C₁-C₄ alkyl, C₁-C₄ haloalkyl, —O(C₁-C₄ alkyl), and —O(C₁-C₄ haloalkyl);
R^{K} is selected from the group consisting of: —NH(C₁₋₄ alkyl); unsubstituted benzothiophenyl; and substituted pyridinyl; wherein the substituted pyridinyl is substituted with one or more substituents Q, wherein each Q is independently selected from the group consisting of: —OH, C₁₋₄ alkyl, C₁₋₄ haloalkyl, halo, cyano, —O—(C₁₋₄ alkyl), and —O—(C₁₋₄ haloalkyl); and
R^{g} is hydrogen or —N(C₁₋₄ alkyl)₂.

16. The compound of claim 13, wherein:
R^{a} is hydrogen;
R^{b} is —(CH₂CH₂)—R^{c};
R^{c} is selected from the group consisting of: substituted phenyl and unsubstituted indolyl; wherein the substituted phenyl is substituted with one substituent E, wherein E is —OH;
R^{K} is selected from the group consisting of: —NH(sec-butyl); unsubstituted benzothiohenyl, and substituted pyridinyl; wherein the substituted pyridinyl is substituted with one or more substituents Q, wherein each Q is independently selected from the group consisting of: C₁₋₄ alkyl, halo, and cyano; and
R^{g} is hydrogen or —N(CH₃)₂.

17. (canceled)

18. The compound of claim 1, wherein the compound of Formula (I) has the structure of Formula (I-D): 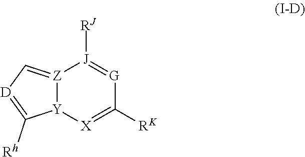
including pharmaceutically acceptable salts thereof, wherein:
R^{J} is —NR^{a}R^{b},
R^{a} is hydrogen or C₁-C₄ alkyl;
R^{b} is R^{c} or —(C₁₋₄ alkyl)-R^{c};
R^{c} is selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E is independently selected from the group consisting of: —OH, C₁-C₄ alkyl, C₁-C₄ haloalkyl, —O(C₁-C₄ alkyl), and —O(C₁-C₄ haloalkyl);
R^{K} is selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q is independently selected from the group consisting of: —OH, C₁₋₄ alkyl, C₁₋₄ haloalkyl, halo, cyano, —O—(C₁₋₄ alkyl), and —O—(C₁₋₄ haloalkyl);
R^{h} is hydrogen or C₁₋₄ alkyl;
D is N or CH;
Y is N;
Z is C; and
X is N or CH.

19. The compound of claim 18, wherein:
R^{a} is hydrogen;
R^{b} is —(C₁₋₄ alkyl)-R^{c};
R^{c} is selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E is independently selected from the group consisting of: —OH, C₁-C₄ alkyl, C₁-C₄ haloalkyl, —O(C₁-C₄ alkyl), and —O(C₁-C₄ haloalkyl);
R^{K} is selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q is independently selected from the group consisting of: —OH, C₁₋₄ alkyl, C₁₋₄ haloalkyl, halo, cyano, —O—(C₁₋₄ alkyl), and —O—(C₁₋₄ haloalkyl); and
R^{h} is hydrogen or C₁₋₄ alkyl.

20. The compound of claim 18, wherein:
R^{a} is hydrogen;
R^{b} is —(C₁-C₄ alkyl)-R^{c};
R^{c} is selected from the group consisting of: substituted phenyl and unsubstituted indolyl; wherein the substituted phenyl is substituted with one or more substituents E, wherein each E is independently selected from the group consisting of: —OH, C₁-C₄ alkyl, C₁-C₄ haloalkyl, —O(C₁-C₄ alkyl), and —O(C₁-C₄ haloalkyl);
R^{K} is unsubstituted benzothiophenyl; and
R^{h} is hydrogen or C₁₋₄ alkyl.

21. The compound of claim 18, wherein:
R^{a} is hydrogen;
R^{b} is —(CH₂—CH₂)—R^{c};
R^{c} is selected from the group consisting of: substituted phenyl and unsubstituted indolyl; wherein the substituted phenyl is substituted with one substituent E, wherein E is —OH;
R^{K} is unsubstituted benzothiophenyl; and
R^{h} is hydrogen or c1-4 alkyl.

22. (canceled)

23. A compound, or pharmaceutically acceptable salt thereof, selected from:
N-(2-(1H-indol-3-yl)ethyl)-7-isopropyl-2-(5-methylpyridin-3-yl)thieno[3,2-d]pyrimidin-4-amine
5-(4-((2-(1H-indol-3-yl)ethyl)amino)-7-isopropylthieno [3,2-d]pyrimidin-2-yl)nicotinonitrile;
N-(2-(1H-indol-3-yl)ethyl)-2-(5-fluoropyridin-3-yl)-7-isopropylthieno[3,2-d]pyrimidin-4-amine
4-(2-((2-(benzo[b]thiophen-3-yl)-7-isopropylthieno [3,2-d]pyrimidin-4-yl)amino)ethyl)phenol;
N-(2-(1H-indol-3-yl)ethyl)-2-(5-fluoropyridin-3-yl)furo[3,2-d]pyrimidin-4-amine;
N-(2-(1H-indol-3-yl)ethyl)-2-(5-methylpyridin-3-yl)furo[3,2-d]pyrimidin-4-amine;
5-(4-((2-(1H-indol-3-yl)ethyl)amino)furo[3,2-d]pyrimidin-2-yl)nicotinonitrile;
3-((2-(benzo[b]thiophen-3-yl)-9-isopropyl-9H-purin-6-yl)oxy)propanamide;
3-(2-(benzo[b]thiophen-3-yl)-9-isopropyl-6-oxo-6,9-dihydro-1^{H}-purin-1-yl)propanamide;
2-(benzo[b]thiophen-3-yl)-4-((4-hydroxyphenethypamino)-7-isopropyl-7H-pyrrolo[2,3-d]pyrimidine-5-carbonitrile;
N-(2-(1H-indol-3-yl)ethyl)-2-methyl-6-phenylthieno[2,3-d]pyrimidin-4-amine;
N-(2-(1H-indol-3-yl)ethyl)-6-(4-fluorophenyl)thieno[2,3-d]pyrimidin-4-amine;
4-(2-((2-(benzo[b]thiophen-3-yl)-8-(dimethylamino)pyrimido[5,4-d]pyrimidin-4-yl)amino)ethyl)phenol;
N-(2-(1H-indol-3-yl)ethyl)-2-(5-fluoropyridin-3-yl)quinazolin-4-amine;
5-(4-((2-(1H-indol-3-yl)ethyl)amino)quinazolin-2-yl)nicotinonitrile;
N⁴-(2-(1H-indol-3-yl)ethyl)-N²-(sec-butyl)quinazoline-2,4-diamine;
N-(2-(1H-indol-3-yl)ethyl)-6-(benzo[b]thiophen-3-yl)-3-isopropylimidazo[1,5-a]pyrazin-8-amine.
4-(2-((6-(benzo[b]thiophen-3-yl)-3-isopropylimidazo[1,5-a]pyrazin-8-yl)amino)ethyl)phenol;
5-(2-((2-(1H-indol-3-yl)ethyl)amino)-6-(sec-butylamino)pyrimidin-4-yl)nicotinonitrile;
4-(2-((2-(benzo[b]thiophen-3-yl)-6-(isopropylamino)pyrimidin-4-yl)amino)ethyl)phenol;
4-(2-((2-(benzo[b]thiophen-3-yl)-7-isopropyl-6,7-dihydro-5H-pyrrolo[2,3-a]pyrimidin-4-yl)amino)ethyl)phenol; and
2-(benzo[b]thiophen-3-yl)-4-((4-hydroxyphenethyl)amino)-7-isopropyl-5,7-dihydro-6H-pyrrolo[2,3-d]pyrimidin-6-one .

24. (canceled)

25. A method of promoting the expansion and/or proliferation of hematopoietic stem cells, comprising:
contacting said hematopoietic stem cells and/or progenitor cells with a compound of Formula (I);
wherein said contacting increases and/or expands the number of stem cells and/or progenitor cells; and
wherein the compound of Formula (I) has the following structure: 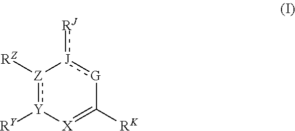
including pharmaceutically acceptable salts thereof, wherein:
each [symbol] independently represents a single bond or a double bond;
R^{J} is selected from the group consisting of —NR^{a}R^{b}, —OR^{b}, and ═O, wherein if R^{J} is ═O, then [symbol] joining G and J represents a single bond and G is N and the N is substituted with R^{G}; otherwise [symbol] joining G and J represents a double bond and G is N;
R^{a} is hydrogen or C₁-C₄ alkyl;
R^{b} is R^{c} or —(C₁-C₄ alkyl)-R^{c};
R^{c} is selected from the group consisting of: —OH, —O(C₁-C₄ alkyl), —O(C₁-C₄ haloalkyl); —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E is independently selected from the group consisting of: —OH, C₁-C₄ alkyl, C₁-C₄ haloalkyl, —O(C₁-C₄ alkyl), and —O(C₁-C₄ haloalkyl);
R^{K} is selected from the group consisting of: hydrogen, unsubstituted C₁₋₆ alkyl; substituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q is independently selected from the group consisting of: —OH, C₁₋₄ alkyl, C₁₋₄ haloalkyl, halo, cyano, —O—(C₁₋₄ alkyl), and —O—(C₁₋₄ haloalkyl);
R^{G} is selected from the group consisting of hydrogen, C₁₋₄ alkyl, and —(C₁₋₄ alkyl)-C(═O)NH₂;
R^{Y} and R^{Z} are each independently absent or selected from the group consisting of: hydrogen, halo, C₁₋₆ alkyl, —OH, —O—(C₁₋₄ alkyl), —NH(C₁₋₄ alkyl), and —N(C₁₋₄ alkyl)₂;
or R^{Y} and R^{Z} taken together with the atoms to which they are attached are joined together to form a ring selected from: 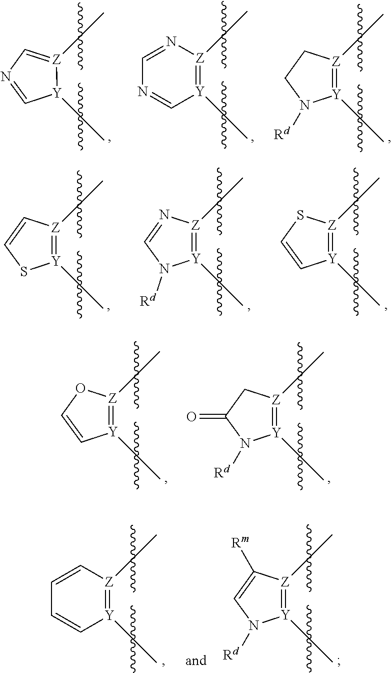
wherein said ring is optionally substituted with one, two, or three groups independently selected from C₁₋₄ alkyl, C₁₋₄ haloalkyl, halo, cyano, —OH, —O—(C₁₋₄ alkyl), —N(C₁₋₄ alkyl)₂, unsubstituted C₆-C₁₀ aryl, C₆-C₁₀ aryl substituted with 1-5 halo atoms, and —O—(C₁₋₄ haloalkyl); and wherein if R^{Y} and R^{Z} taken together forms 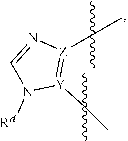
then R^{J} is —OR^{b} or ═O;
R^{d} is hydrogen or C₁-C₄ alkyl;
R^{m} is selected from the group consisting of C₁₋₄ alkyl, halo, and cyano;
J is C; and
X, Y, and Z are each independently N or C, wherein the valency of any carbon atom is filled as needed with hydrogen atoms.

26.-56. (canceled)
